SEQUENCE LISTING (84 sequences)

SEQ ID NO: 1 - protein, 315 aa, synthetic construct
MLKMESTQQMAVSIINSSFEAAVVAATSALENMGIEYDYQDIYSRVKNKFDFVMDDSGVKNNLIGKAITIDQALNNKFGSAIRNRNWLADTSRAAKLDEDVNKLRMMLSSKGIDQKMRVLNACFSVKRIPGKSSSIIKCTKLMRDKLERGEVEVDDSFVDEKMEVDTIDWKSRYEQLEQRFESLKSRVNEKYNNWVLKARKMNENMHSLQNVISQQQAHIAELQVYNNKLERDLQNKIGSLTSSIEWYLRSMELDPEIKADIEQQINSIDAINPLHAFDDLESVIRNLISDYDKLFLMFKGLIQRCNYQYSFGCE

SEQ ID NO: 2 - protein, 1046 aa, synthetic construct
MLKMESTQQMAVSIINSSFEAAVVAATSALENMGIEYDYQDIYSRVKNKFDFVMDDSGVKNNLIGKAITIDQALNNKFGSAIRNRNWLADTSRAAKLDEDVNKLRMMLSSKGIDQKMRVLNACFSVKRIPGKSSSIIKCTKLMRDKLERGEVEVDDSFVDEKMEVDTIDWKSRYEQLEQRFESLKSRVNEKYNNWVLKARKMNENMHSLQNVISQQQAHIAELQVYNNKLERDLQNKIGSLTSSIEWYLRSMELDPEIKADIEQQINSIDAINPLHAFDDLESVIRNLISDYDKLFLMFKGLIQRCNYQYSFGCEGAGATNFSLLKQAGDVEENPGPDYKDHDGDYKDHDIDYKDDDDKASVFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 3 - protein, 1030 aa, synthetic construct
MLKMESTQQMAVSIINSSFEAAVVAATSALENMGIEYDYQDIYSRVKNKFDFVMDDSGVKNNLIGKAITIDQALNNKFGSAIRNRNWLADTSRAAKLDEDVNKLRMMLSSKGIDQKMRVLNACFSVKRIPGKSSSIIKCTKLMRDKLERGEVEVDDSFVDEKMEVDTIDWKSRYEQLEQRFESLKSRVNEKYNNWVLKARKMNENMHSLQNVISQQQAHIAELQVYNNKLERDLQNKIGSLTSSIEWYLRSMELDPEIKADIEQQINSIDAINPLHAFDDLESVIRNLISDYDKLFLMFKGLIQRCNYQYSFGCEGAGATNFSLLKQAGDVEENPGPVFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARDYKDDDDK

SEQ ID NO: 4 - protein, 1052 aa, synthetic construct
MLKMESTQQMAVSIINSSFEAAVVAATSALENMGIEYDYQDIYSRVKNKFDFVMDDSGVKNNLIGKAITIDQALNNKFGSAIRNRNWLADTSRAAKLDEDVNKLRMMLSSKGIDQKMRVLNACFSVKRIPGKSSSIIKCTKLMRDKLERGEVEVDDSFVDEKMEVDTIDWKSRYEQLEQRFESLKSRVNEKYNNWVLKARKMNENMHSLQNVISQQQAHIAELQVYNNKLERDLQNKIGSLTSSIEWYLRSMELDPEIKADIEQQINSIDAINPLHAFDDLESVIRNLISDYDKLFLMFKGLIQRCNYQYSFGCEGAGATNFSLLKQAGDVEENPGPDYKDHDGDYKDHDIDYKDDDDKASVFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARHHHHHH

SEQ ID NO: 5 - protein, 504 aa, synthetic construct
MLKMESTQQMAVSIINSSFEAAVVAATSALENMGIEYDYQDIYSRVKNKFDFVMDDSGVKNNLIGKAITIDQALNNKFGSAIRNRNWLADTSRAAKLDEDVNKLRMMLSSKGIDQKMRVLNACFSVKRIPGKSSSIIKCTKLMRDKLERGEVEVDDSFVDEKMEVDTIDWKSRYEQLEQRFESLKSRVNEKYNNWVLKARKMNENMHSLQNVISQQQAHIAELQVYNNKLERDLQNKIGSLTSSIEWYLRSMELDPEIKADIEQQINSIDAINPLHAFDDLESVIRNLISDYDKLFLMFKGLIQRCNYQYSFGCEGAGATNFSLLKQAGDVEENPGPDYKDHDGDYKDHDIDYKDDDDKASTTQLSAVNICNFRGDVTHIVGSHDYTMNLASQNWSNYDPTEEIPAPLGTPDFVGKIQGLLTQTTRADGSTRAHKATVSTGSVHFTPKLGSVQFTTDTNNDFQTGQNTKFTPVGVIQDGDHHQNEPQQWVLPNYSGRTGHNVHL

SEQ ID NO: 6 - protein, 676 aa, synthetic construct
MLKMESTQQMAVSIINSSFEAAVVAATSALENMGIEYDYQDIYSRVKNKFDFVMDDSGVKNNLIGKAITIDQALNNKFGSAIRNRNWLADTSRAAKLDEDVNKLRMMLSSKGIDQKMRVLNACFSVKRIPGKSSSIIKCTKLMRDKLERGEVEVDDSFVDEKMEVDTIDWKSRYEQLEQRFESLKSRVNEKYNNWVLKARKMNENMHSLQNVISQQQAHIAELQVYNNKLERDLQNKIGSLTSSIEWYLRSMELDPEIKADIEQQINSIDAINPLHAFDDLESVIRNLISDYDKLFLMFKGLIQRCNYQYSFGCEGAGATNFSLLKQAGDVEENPGPDYKDHDGDYKDHDIDYKDDDDKASKPFTVPILTVEEMSNSRFPIPLEKLYTGPSSAFVVQPQNGRCTTDGVLLGTTQLSAVNICNFRGDVTHIVGSHDYTMNLASQNWSNYDPTEEIPAPLGTPDFVGKIQGLLTQTTRADGSTRAHKATVSTGSVHFTPKLGSVQFTTDTNNDFQTGQNTKFTPVGVIQDGDHHQNEPQQWVLPNYSGRTGHNVHLAPAVAPTFPGEQLLFFRSTMPGCSGYPNMNLDCLLPQEWVLHFYQEAAPAQSDVALLRFVNPDTGRVLFECKLHKSGYITVAHTGPYDLVIPPNGYFRFDSWVNQFYTLAPMGNGTGRRRAL

SEQ ID NO: 7 - protein, 900 aa, synthetic construct
MLKMESTQQMAVSIINSSFEAAVVAATSALENMGIEYDYQDIYSRVKNKFDFVMDDSGVKNNLIGKAITIDQALNNKFGSAIRNRNWLADTSRAAKLDEDVNKLRMMLSSKGIDQKMRVLNACFSVKRIPGKSSSIIKCTKLMRDKLERGEVEVDDSFVDEKMEVDTIDWKSRYEQLEQRFESLKSRVNEKYNNWVLKARKMNENMHSLQNVISQQQAHIAELQVYNNKLERDLQNKIGSLTSSIEWYLRSMELDPEIKADIEQQINSIDAINPLHAFDDLESVIRNLISDYDKLFLMFKGLIQRCNYQYSFGCEGAGATNFSLLKQAGDVEENPGPDYKDHDGDYKDHDIDYKDDDDKASMKMASSDANPSDGSAANLVPEVNNEVMALEPVVGAAIAAPVAGQQNIIDPWIRNNFVQAPGGEFTVSPRNAPGEILWSAPLGPDLNPYLSHLSRMYNGYAGGFEVQVILAGNAFTAGKVIFAAVPPNFPTEGLSPSQVTMFPHIIVDVRQLEPVLIPLPDVRNNFYHYNQSHDSTLKLIAMLYTPLRANNAGDDVFTVSCRVLTRPSPDFDFIFLVPPTVESRTKPFTVPILTVEEMSNSRFPIPLEKLYTGPSSAFVVQPQNGRCTTDGVLLGTTQLSAVNICNFRGDVTHIVGSHDYTMNLASQNWSNYDPTEEIPAPLGTPDFVGKIQGLLTQTTRADGSTRAHKATVSTGSVHFTPKLGSVQFTTDTNNDFQTGQNTKFTPVGVIQDGDHHQNEPQQWVLPNYSGRTGHNVHLAPAVAPTFPGEQLLFFRSTMPGCSGYPNMNLDCLLPQEWVLHFYQEAAPAQSDVALLRFVNPDTGRVLFECKLHKSGYITVAHTGPYDLVIPPNGYFRFDSWVNQFYTLAPMGNGTGRRRAL

SEQ ID NO: 8 - protein, 886 aa, synthetic construct
MLKMESTQQMAVSIINSSFEAAVVAATSALENMGIEYDYQDIYSRVKNKFDFVMDDSGVKNNLIGKAITIDQALNNKFGSAIRNRNWLADTSRAAKLDEDVNKLRMMLSSKGIDQKMRVLNACFSVKRIPGKSSSIIKCTKLMRDKLERGEVEVDDSFVDEKMEVDTIDWKSRYEQLEQRFESLKSRVNEKYNNWVLKARKMNENMHSLQNVISQQQAHIAELQVYNNKLERDLQNKIGSLTSSIEWYLRSMELDPEIKADIEQQINSIDAINPLHAFDDLESVIRNLISDYDKLFLMFKGLIQRCNYQYSFGCEGAGATNFSLLKQAGDVEENPGPASVKMASSDANPSDGSAANLVPEVNNEVMALEPVVGAAIAAPVAGQQNIIDPWIRNNFVQAPGGEFTVSPRNAPGEILWSAPLGPDLNPYLSHLSRMYNGYAGGFEVQVILAGNAFTAGKVIFAAVPPNFPTEGLSPSQVTMFPHIIVDVRQLEPVLIPLPDVRNNFYHYNQSHDSTLKLIAMLYTPLRANNAGDDVFTVSCRVLTRPSPDFDFIFLVPPTVESRTKPFTVPILTVEEMSNSRFPIPLEKLYTGPSSAFVVQPQNGRCTTDGVLLGTTQLSAVNICNFRGDVTHIVGSHDYTMNLASQNWSNYDPTEEIPAPLGTPDFVGKIQGLLTQTTRADGSTRAHKATVSTGSVHFTPKLGSVQFTTDTNNDFQTGQNTKFTPVGVIQDGDHHQNEPQQWVLPNYSGRTGHNVHLAPAVAPTFPGEQLLFFRSTMPGCSGYPNMNLDCLLPQEWVLHFYQEAAPAQSDVALLRFVNPDTGRVLFECKLHKSGYITVAHTGPYDLVIPPNGYFRFDSWVNQFYTLAPMGNGTGRRRALDYKDDDDK

SEQ ID NO: 9 - protein, 660 aa, synthetic construct
MLKMESTQQMAVSIINSSFEAAVVAATSALENMGIEYDYQDIYSRVKNKFDFVMDDSGVKNNLIGKAITIDQALNNKFGSAIRNRNWLADTSRAAKLDEDVNKLRMMLSSKGIDQKMRVLNACFSVKRIPGKSSSIIKCTKLMRDKLERGEVEVDDSFVDEKMEVDTIDWKSRYEQLEQRFESLKSRVNEKYNNWVLKARKMNENMHSLQNVISQQQAHIAELQVYNNKLERDLQNKIGSLTSSIEWYLRSMELDPEIKADIEQQINSIDAINPLHAFDDLESVIRNLISDYDKLFLMFKGLIQRCNYQYSFGCEGAGATNFSLLKQAGDVEENPGPASKPFTVPILTVEEMSNSRFPIPLEKLYTGPSSAFVVQPQNGRCTTDGVLLGTTQLSAVNICNFRGDVTHIVGSHDYTMNLASQNWSNYDPTEEIPAPLGTPDFVGKIQGLLTQTTRADGSTRAHKATVSTGSVHFTPKLGSVQFTTDTNNDFQTGQNTKFTPVGVIQDGDHHQNEPQQWVLPNYSGRTGHNVHLAPAVAPTFPGEQLLFFRSTMPGCSGYPNMNLDCLLPQEWVLHFYQEAAPAQSDVALLRFVNPDTGRVLFECKLHKSGYITVAHTGPYDLVIPPNGYFRFDSWVNQFYTLAPMGNGTGRRRALHHHHHH

SEQ ID NO: 10 - protein, 888 aa, synthetic construct
MLKMESTQQMAVSIINSSFEAAVVAATSALENMGIEYDYQDIYSRVKNKFDFVMDDSGVKNNLIGKAITIDQALNNKFGSAIRNRNWLADTSRAAKLDEDVNKLRMMLSSKGIDQKMRVLNACFSVKRIPGKSSSIIKCTKLMRDKLERGEVEVDDSFVDEKMEVDTIDWKSRYEQLEQRFESLKSRVNEKYNNWVLKARKMNENMHSLQNVISQQQAHIAELQVYNNKLERDLQNKIGSLTSSIEWYLRSMELDPEIKADIEQQINSIDAINPLHAFDDLESVIRNLISDYDKLFLMFKGLIQRCNYQYSFGCEGAGATNFSLLKQAGDVEENPGPVKMASSDANPSDGSAANLVPEVNNEVMALEPVVGAAIAAPVAGQQNIIDPWIRNNFVQAPGGEFTVSPRNAPGEILWSAPLGPDLNPYLSHLSRMYNGYAGGFEVQVILAGNAFTAGKVIFAAVPPNFPTEGLSPSQVTMFPHIIVDVRQLEPVLIPLPDVRNNFYHYNQSHDSTLKLIAMLYTPLRANNAGDDVFTVSCRVLTRPSPDFDFIFLVPPTVESRTKPFTVPILTVEEMSNSRFPIPLEKLYTGPSSAFVVQPQNGRCTTDGVLLGTTQLSAVNICNFRGDVTHIVGSHDYTMNLASQNWSNYDPTEEIPAPLGTPDFVGKIQGLLTQTTRADGSTRAHKATVSTGSVHFTPKLGSVQFTTDTNNDFQTGQNTKFTPVGVIQDGDHHQNEPQQWVLPNYSGRTGHNVHLAPAVAPTFPGEQLLFFRSTMPGCSGYPNMNLDCLLPQEWVLHFYQEAAPAQSDVALLRFVNPDTGRVLFECKLHKSGYITVAHTGPYDLVIPPNGYFRFDSWVNQFYTLAPMGNGTGRRRALLVPRGSHHHHHH

SEQ ID NO: 11 - protein, 882 aa, synthetic construct
MLKMESTQQMAVSIINSSFEAAVVAATSALENMGIEYDYQDIYSRVKNKFDFVMDDSGVKNNLIGKAITIDQALNNKFGSAIRNRNWLADTSRAAKLDEDVNKLRMMLSSKGIDQKMRVLNACFSVKRIPGKSSSIIKCTKLMRDKLERGEVEVDDSFVDEKMEVDTIDWKSRYEQLEQRFESLKSRVNEKYNNWVLKARKMNENMHSLQNVISQQQAHIAELQVYNNKLERDLQNKIGSLTSSIEWYLRSMELDPEIKADIEQQINSIDAINPLHAFDDLESVIRNLISDYDKLFLMFKGLIQRCNYQYSFGCEGAGATNFSLLKQAGDVEENPGPVKMASSDANPSDGSAANLVPEVNNEVMALEPVVGAAIAAPVAGQQNIIDPWIRNNFVQAPGGEFTVSPRNAPGEILWSAPLGPDLNPYLSHLSRMYNGYAGGFEVQVILAGNAFTAGKVIFAAVPPNFPTEGLSPSQVTMFPHIIVDVRQLEPVLIPLPDVRNNFYHYNQSHDSTLKLIAMLYTPLRANNAGDDVFTVSCRVLTRPSPDFDFIFLVPPTVESRTKPFTVPILTVEEMSNSRFPIPLEKLYTGPSSAFVVQPQNGRCTTDGVLLGTTQLSAVNICNFRGDVTHIVGSHDYTMNLASQNWSNYDPTEEIPAPLGTPDFVGKIQGLLTQTTRADGSTRAHKATVSTGSVHFTPKLGSVQFTTDTNNDFQTGQNTKFTPVGVIQDGDHHQNEPQQWVLPNYSGRTGHNVHLAPAVAPTFPGEQLLFFRSTMPGCSGYPNMNLDCLLPQEWVLHFYQEAAPAQSDVALLRFVNPDTGRVLFECKLHKSGYITVAHTGPYDLVIPPNGYFRFDSWVNQFYTLAPMGNGTGRRRALHHHHHH

SEQ ID NO: 12 - DNA, 1105 bp, synthetic construct
  source 1..1105: mol_type = other DNA
ggcatttaatgcttttcagtggttgatgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaataaccattttgatacatgttgaacaatcaaatacagtgttagtatgttgtcatctatgcataaccctctatgagcacaatagttaaaagctaacactgtcaaaaacctaaatggctataggggcgttatgtggcc

SEQ ID NO: 13 - DNA, 3298 bp, synthetic construct
  source 1..3298: mol_type = other DNA
ggcatttaatgcttttcagtggttgatgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaaggcgccggcgcaacgaacttctctctgttaaagcaagcaggagacgtggaagaaaaccccggtcctgactacaaagaccatgacggtgattataaagatcatgatatcgattacaaggatgacgatgacaaggctagcgtgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgttgaccattttgatacatgttgaacaatcaaatacagtgttagtatgttgtcatctatgcataaccctctatgagcacaatagttaaaagctaacactgtcaaaaacctaaatggctataggggcgttatgtggcc

SEQ ID NO: 14 - DNA, 3250 bp, synthetic construct
  source 1..3250: mol_type = other DNA
ggcatttaatgcttttcagtggttgatgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaaggcgccggcgcaacgaacttctctctgttaaagcaagcaggagacgtggaagaaaaccccggtcctgtgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtgattacaaggatgacgatgacaagtgaccattttgatacatgttgaacaatcaaatacagtgttagtatgttgtcatctatgcataaccctctatgagcacaatagttaaaagctaacactgtcaaaaacctaaatggctataggggcgttatgtggcc

SEQ ID NO: 15 - DNA, 3316 bp, synthetic construct
  source 1..3316: mol_type = other DNA
ggcatttaatgcttttcagtggttgatgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaaggcgccggcgcaacgaacttctctctgttaaagcaagcaggagacgtggaagaaaaccccggtcctgactacaaagaccatgacggtgattataaagatcatgatatcgattacaaggatgacgatgacaaggctagcgtgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtcaccaccaccaccaccactgaccattttgatacatgttgaacaatcaaatacagtgttagtatgttgtcatctatgcataaccctctatgagcacaatagttaaaagctaacactgtcaaaaacctaaatggctataggggcgttatgtggcc

SEQ ID NO: 16 - DNA, 1515 bp, synthetic construct
  source 1..1515: mol_type = other DNA
atgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaaggcgccggcgcaacgaacttctctctgttaaagcaagcaggagacgtggaagaaaaccccggtcctgactacaaagaccatgacggtgattataaagatcatgatatcgattacaaggatgacgatgacaaggctagcactacccagctgtcagctgtcaacatctgtaactttaggggggatgtcacccatattgtgggcagccatgattatacaatgaatctggcttcccaaaattggagcaattatgacccaacagaagaaatcccagcccccctgggaacaccagattttgtggggaagatccaaggcctgctcacccagaccacaagagcggatggctcgacccgtgcccacaaagctacagtgagcactgggagtgtccacttcactccaaagctgggtagtgttcaattcaccactgacacaaacaatgatttccaaactggccaaaacacgaaattcaccccagttggcgtcatccaggacggtgatcaccatcagaatgagccccaacaatgggtactcccaaattactcaggtagaactggtcataatgtgcacctgtga

SEQ ID NO: 17 - DNA, 2031 bp, synthetic construct
  source 1..2031: mol_type = other DNA
atgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaaggcgccggcgcaacgaacttctctctgttaaagcaagcaggagacgtggaagaaaaccccggtcctgactacaaagaccatgacggtgattataaagatcatgatatcgattacaaggatgacgatgacaaggctagcaaaccattcaccgtcccaatcttaactgttgaggaaatgtccaattcaagattccccattcctttggaaaagttgtacacgggtcctagcagtgcttttgttgtccaaccacaaaatggcagatgcacgactgatggcgtgctcttaggtactacccagctgtcagctgtcaacatctgtaactttaggggggatgtcacccatattgtgggcagccatgattatacaatgaatctggcttcccaaaattggagcaattatgacccaacagaagaaatcccagcccccctgggaacaccagattttgtggggaagatccaaggcctgctcacccagaccacaagagcggatggctcgacccgtgcccacaaagctacagtgagcactgggagtgtccacttcactccaaagctgggtagtgttcaattcaccactgacacaaacaatgatttccaaactggccaaaacacgaaattcaccccagttggcgtcatccaggacggtgatcaccatcagaatgagccccaacaatgggtactcccaaattactcaggtagaactggtcataatgtgcacctggcccctgccgttgcccccacttttccgggtgagcaactccttttctttagatccactatgcccggatgtagcgggtatcccaacatgaatttggattgcctactcccccaggaatgggtgctgcacttctaccaggaagcagctccagcacaatccgatgtggctctgctgagatttgtgaatccagacacaggtagggttctgtttgagtgcaagctccataaatcaggctatatcacagtggctcacaccggcccgtatgacttggttatcccccccaatggttattttagatttgattcctgggtcaaccagttctacacacttgcccccatgggaaatggaacggggcgcaggcgtgcattataa

SEQ ID NO: 18 - DNA, 2703 bp, synthetic construct
  source 1..2703: mol_type = other DNA
atgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaaggcgccggcgcaacgaacttctctctgttaaagcaagcaggagacgtggaagaaaaccccggtcctgactacaaagaccatgacggtgattataaagatcatgatatcgattacaaggatgacgatgacaaggctagcatgaagatggcgtcgagtgacgccaacccatctgatgggtccgcagccaacctcgtcccagaggtcaacaatgaggttatggctctggagcccgttgttggtgccgctattgcggcacctgtagcgggccaacaaaatataattgacccctggattagaaataattttgtacaagcccctggtggagagtttacagtgtcccctagaaacgctccaggtgagatactatggagcgcgcccttgggccctgatttgaacccctatctttctcatttgtccagaatgtacaatggttatgcaggcggtttcgaagtgcaagtaatcctcgcggggaacgcgttcaccgccgggaaagttatatttgcagcagttccaccaaactttccaactgaaggcttaagccccagccaggttactatgttcccccatataattgtagatgttaggcaattggaacctgtgttgatccccctacctgatgttaggaataatttctatcattacaatcaatcacatgattctacccttaagttgatagcaatgttgtatacaccactcagggctaataatgccggggacgatgtcttcacagtctcttgtcgagttctcacgaggccatcccccgattttgatttcatattcttggtgccacccacagttgaatcaagaactaaaccattcaccgtcccaatcttaactgttgaggaaatgtccaattcaagattccccattcctttggaaaagttgtacacgggtcctagcagtgcttttgttgtccaaccacaaaatggcagatgcacgactgatggcgtgctcttaggtactacccagctgtcagctgtcaacatctgtaactttaggggggatgtcacccatattgtgggcagccatgattatacaatgaatctggcttcccaaaattggagcaattatgacccaacagaagaaatcccagcccccctgggaacaccagattttgtggggaagatccaaggcctgctcacccagaccacaagagcggatggctcgacccgtgcccacaaagctacagtgagcactgggagtgtccacttcactccaaagctgggtagtgttcaattcaccactgacacaaacaatgatttccaaactggccaaaacacgaaattcaccccagttggcgtcatccaggacggtgatcaccatcagaatgagccccaacaatgggtactcccaaattactcaggtagaactggtcataatgtgcacctggcccctgccgttgcccccacttttccgggtgagcaactccttttctttagatccactatgcccggatgtagcgggtatcccaacatgaatttggattgcctactcccccaggaatgggtgctgcacttctaccaggaagcagctccagcacaatccgatgtggctctgctgagatttgtgaatccagacacaggtagggttctgtttgagtgcaagctccataaatcaggctatatcacagtggctcacaccggcccgtatgacttggttatcccccccaatggttattttagatttgattcctgggtcaaccagttctacacacttgcccccatgggaaatggaacggggcgcaggcgtgcattataa

SEQ ID NO: 19 - DNA, 2661 bp, synthetic construct
  source 1..2661: mol_type = other DNA
atgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaaggcgccggcgcaacgaacttctctctgttaaagcaagcaggagacgtggaagaaaaccccggtcctgctagcgtgaagatggcgtcgagtgacgccaacccatctgatgggtccgcagccaacctcgtcccagaggtcaacaatgaggttatggctctggagcccgttgttggtgccgctattgcggcacctgtagcgggccaacaaaatataattgacccctggattagaaataattttgtacaagcccctggtggagagtttacagtgtcccctagaaacgctccaggtgagatactatggagcgcgcccttgggccctgatttgaacccctatctttctcatttgtccagaatgtacaatggttatgcaggcggtttcgaagtgcaagtaatcctcgcggggaacgcgttcaccgccgggaaagttatatttgcagcagttccaccaaactttccaactgaaggcttaagccccagccaggttactatgttcccccatataattgtagatgttaggcaattggaacctgtgttgatccccctacctgatgttaggaataatttctatcattacaatcaatcacatgattctacccttaagttgatagcaatgttgtatacaccactcagggctaataatgccggggacgatgtcttcacagtctcttgtcgagttctcacgaggccatcccccgattttgatttcatattcttggtgccacccacagttgaatcaagaactaaaccattcaccgtcccaatcttaactgttgaggaaatgtccaattcaagattccccattcctttggaaaagttgtacacgggtcctagcagtgcttttgttgtccaaccacaaaatggcagatgcacgactgatggcgtgctcttaggtactacccagctgtcagctgtcaacatctgtaactttaggggggatgtcacccatattgtgggcagccatgattatacaatgaatctggcttcccaaaattggagcaattatgacccaacagaagaaatcccagcccccctgggaacaccagattttgtggggaagatccaaggcctgctcacccagaccacaagagcggatggctcgacccgtgcccacaaagctacagtgagcactgggagtgtccacttcactccaaagctgggtagtgttcaattcaccactgacacaaacaatgatttccaaactggccaaaacacgaaattcaccccagttggcgtcatccaggacggtgatcaccatcagaatgagccccaacaatgggtactcccaaattactcaggtagaactggtcataatgtgcacctggcccctgccgttgcccccacttttccgggtgagcaactccttttctttagatccactatgcccggatgtagcgggtatcccaacatgaatttggattgcctactcccccaggaatgggtgctgcacttctaccaggaagcagctccagcacaatccgatgtggctctgctgagatttgtgaatccagacacaggtagggttctgtttgagtgcaagctccataaatcaggctatatcacagtggctcacaccggcccgtatgacttggttatcccccccaatggttattttagatttgattcctgggtcaaccagttctacacacttgcccccatgggaaatggaacggggcgcaggcgtgcattagattacaaggatgacgatgacaagtaa

SEQ ID NO: 20 - DNA, 1983 bp, synthetic construct
  source 1..1983: mol_type = other DNA
atgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaaggcgccggcgcaacgaacttctctctgttaaagcaagcaggagacgtggaagaaaaccccggtcctgctagcaaaccattcaccgtcccaatcttaactgttgaggaaatgtccaattcaagattccccattcctttggaaaagttgtacacgggtcctagcagtgcttttgttgtccaaccacaaaatggcagatgcacgactgatggcgtgctcttaggtactacccagctgtcagctgtcaacatctgtaactttaggggggatgtcacccatattgtgggcagccatgattatacaatgaatctggcttcccaaaattggagcaattatgacccaacagaagaaatcccagcccccctgggaacaccagattttgtggggaagatccaaggcctgctcacccagaccacaagagcggatggctcgacccgtgcccacaaagctacagtgagcactgggagtgtccacttcactccaaagctgggtagtgttcaattcaccactgacacaaacaatgatttccaaactggccaaaacacgaaattcaccccagttggcgtcatccaggacggtgatcaccatcagaatgagccccaacaatgggtactcccaaattactcaggtagaactggtcataatgtgcacctggcccctgccgttgcccccacttttccgggtgagcaactccttttctttagatccactatgcccggatgtagcgggtatcccaacatgaatttggattgcctactcccccaggaatgggtgctgcacttctaccaggaagcagctccagcacaatccgatgtggctctgctgagatttgtgaatccagacacaggtagggttctgtttgagtgcaagctccataaatcaggctatatcacagtggctcacaccggcccgtatgacttggttatcccccccaatggttattttagatttgattcctgggtcaaccagttctacacacttgcccccatgggaaatggaacggggcgcaggcgtgcattacaccaccaccaccaccactaa

SEQ ID NO: 21 - DNA, 2667 bp, synthetic construct
  source 1..2667: mol_type = other DNA
atgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaaggcgccggcgcaacgaacttctctctgttaaagcaagcaggagacgtggaagaaaaccccggtcctgtgaagatggcgtcgagtgacgccaacccatctgatgggtccgcagccaacctcgtcccagaggtcaacaatgaggttatggctctggagcccgttgttggtgccgctattgcggcacctgtagcgggccaacaaaatataattgacccctggattagaaataattttgtacaagcccctggtggagagtttacagtgtcccctagaaacgctccaggtgagatactatggagcgcgcccttgggccctgatttgaacccctatctttctcatttgtccagaatgtacaatggttatgcaggcggtttcgaagtgcaagtaatcctcgcggggaacgcgttcaccgccgggaaagttatatttgcagcagttccaccaaactttccaactgaaggcttaagccccagccaggttactatgttcccccatataattgtagatgttaggcaattggaacctgtgttgatccccctacctgatgttaggaataatttctatcattacaatcaatcacatgattctacccttaagttgatagcaatgttgtatacaccactcagggctaataatgccggggacgatgtcttcacagtctcttgtcgagttctcacgaggccatcccccgattttgatttcatattcttggtgccacccacagttgaatcaagaactaaaccattcaccgtcccaatcttaactgttgaggaaatgtccaattcaagattccccattcctttggaaaagttgtacacgggtcctagcagtgcttttgttgtccaaccacaaaatggcagatgcacgactgatggcgtgctcttaggtactacccagctgtcagctgtcaacatctgtaactttaggggggatgtcacccatattgtgggcagccatgattatacaatgaatctggcttcccaaaattggagcaattatgacccaacagaagaaatcccagcccccctgggaacaccagattttgtggggaagatccaaggcctgctcacccagaccacaagagcggatggctcgacccgtgcccacaaagctacagtgagcactgggagtgtccacttcactccaaagctgggtagtgttcaattcaccactgacacaaacaatgatttccaaactggccaaaacacgaaattcaccccagttggcgtcatccaggacggtgatcaccatcagaatgagccccaacaatgggtactcccaaattactcaggtagaactggtcataatgtgcacctggcccctgccgttgcccccacttttccgggtgagcaactccttttctttagatccactatgcccggatgtagcgggtatcccaacatgaatttggattgcctactcccccaggaatgggtgctgcacttctaccaggaagcagctccagcacaatccgatgtggctctgctgagatttgtgaatccagacacaggtagggttctgtttgagtgcaagctccataaatcaggctatatcacagtggctcacaccggcccgtatgacttggttatcccccccaatggttattttagatttgattcctgggtcaaccagttctacacacttgcccccatgggaaatggaacggggcgcaggcgtgcattattggtcccccgtggctcgcaccaccaccaccaccactaa

SEQ ID NO: 22 - DNA, 2649 bp, synthetic construct
  source 1..2649: mol_type = other DNA
atgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaaggcgccggcgcaacgaacttctctctgttaaagcaagcaggagacgtggaagaaaaccccggtcctgtgaagatggcgtcgagtgacgccaacccatctgatgggtccgcagccaacctcgtcccagaggtcaacaatgaggttatggctctggagcccgttgttggtgccgctattgcggcacctgtagcgggccaacaaaatataattgacccctggattagaaataattttgtacaagcccctggtggagagtttacagtgtcccctagaaacgctccaggtgagatactatggagcgcgcccttgggccctgatttgaacccctatctttctcatttgtccagaatgtacaatggttatgcaggcggtttcgaagtgcaagtaatcctcgcggggaacgcgttcaccgccgggaaagttatatttgcagcagttccaccaaactttccaactgaaggcttaagccccagccaggttactatgttcccccatataattgtagatgttaggcaattggaacctgtgttgatccccctacctgatgttaggaataatttctatcattacaatcaatcacatgattctacccttaagttgatagcaatgttgtatacaccactcagggctaataatgccggggacgatgtcttcacagtctcttgtcgagttctcacgaggccatcccccgattttgatttcatattcttggtgccacccacagttgaatcaagaactaaaccattcaccgtcccaatcttaactgttgaggaaatgtccaattcaagattccccattcctttggaaaagttgtacacgggtcctagcagtgcttttgttgtccaaccacaaaatggcagatgcacgactgatggcgtgctcttaggtactacccagctgtcagctgtcaacatctgtaactttaggggggatgtcacccatattgtgggcagccatgattatacaatgaatctggcttcccaaaattggagcaattatgacccaacagaagaaatcccagcccccctgggaacaccagattttgtggggaagatccaaggcctgctcacccagaccacaagagcggatggctcgacccgtgcccacaaagctacagtgagcactgggagtgtccacttcactccaaagctgggtagtgttcaattcaccactgacacaaacaatgatttccaaactggccaaaacacgaaattcaccccagttggcgtcatccaggacggtgatcaccatcagaatgagccccaacaatgggtactcccaaattactcaggtagaactggtcataatgtgcacctggcccctgccgttgcccccacttttccgggtgagcaactccttttctttagatccactatgcccggatgtagcgggtatcccaacatgaatttggattgcctactcccccaggaatgggtgctgcacttctaccaggaagcagctccagcacaatccgatgtggctctgctgagatttgtgaatccagacacaggtagggttctgtttgagtgcaagctccataaatcaggctatatcacagtggctcacaccggcccgtatgacttggttatcccccccaatggttattttagatttgattcctgggtcaaccagttctacacacttgcccccatgggaaatggaacggggcgcaggcgtgcattacaccaccaccaccaccactaa

SEQ ID NO: 23 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 24 - protein, 539 aa, synthetic construct
MKMASSDANPSDGSAANLVPEVNNEVMALEPVVGAAIAAPVAGQQNIIDPWIRNNFVQAPGGEFTVSPRNAPGEILWSAPLGPDLNPYLSHLSRMYNGYAGGFEVQVILAGNAFTAGKVIFAAVPPNFPTEGLSPSQVTMFPHIIVDVRQLEPVLIPLPDVRNNFYHYNQSHDSTLKLIAMLYTPLRANNAGDDVFTVSCRVLTRPSPDFDFIFLVPPTVESRTKPFTVPILTVEEMSNSRFPIPLEKLYTGPSSAFVVQPQNGRCTTDGVLLGTTQLSAVNICNFRGDVTHIVGSHDYTMNLASQNWSNYDPTEEIPAPLGTPDFVGKIQGLLTQTTRADGSTRAHKATVSTGSVHFTPKLGSVQFTTDTNNDFQTGQNTKFTPVGVIQDGDHHQNEPQQWVLPNYSGRTGHNVHLAPAVAPTFPGEQLLFFRSTMPGCSGYPNMNLDCLLPQEWVLHFYQEAAPAQSDVALLRFVNPDTGRVLFECKLHKSGYITVAHTGPYDLVIPPNGYFRFDSWVNQFYTLAPMGNGTGRRRAL

SEQ ID NO: 25 - DNA, 1620 bp, synthetic construct
  source 1..1620: mol_type = other DNA
atgaagatggcgtcgagtgacgccaacccatctgatgggtccgcagccaacctcgtcccagaggtcaacaatgaggttatggctctggagcccgttgttggtgccgctattgcggcacctgtagcgggccaacaaaatataattgacccctggattagaaataattttgtacaagcccctggtggagagtttacagtgtcccctagaaacgctccaggtgagatactatggagcgcgcccttgggccctgatttgaacccctatctttctcatttgtccagaatgtacaatggttatgcaggcggtttcgaagtgcaagtaatcctcgcggggaacgcgttcaccgccgggaaagttatatttgcagcagttccaccaaactttccaactgaaggcttaagccccagccaggttactatgttcccccatataattgtagatgttaggcaattggaacctgtgttgatccccctacctgatgttaggaataatttctatcattacaatcaatcacatgattctacccttaagttgatagcaatgttgtatacaccactcagggctaataatgccggggacgatgtcttcacagtctcttgtcgagttctcacgaggccatcccccgattttgatttcatattcttggtgccacccacagttgaatcaagaactaaaccattcaccgtcccaatcttaactgttgaggaaatgtccaattcaagattccccattcctttggaaaagttgtacacgggtcctagcagtgcttttgttgtccaaccacaaaatggcagatgcacgactgatggcgtgctcttaggtactacccagctgtcagctgtcaacatctgtaactttaggggggatgtcacccatattgtgggcagccatgattatacaatgaatctggcttcccaaaattggagcaattatgacccaacagaagaaatcccagcccccctgggaacaccagattttgtggggaagatccaaggcctgctcacccagaccacaagagcggatggctcgacccgtgcccacaaagctacagtgagcactgggagtgtccacttcactccaaagctgggtagtgttcaattcaccactgacacaaacaatgatttccaaactggccaaaacacgaaattcaccccagttggcgtcatccaggacggtgatcaccatcagaatgagccccaacaatgggtactcccaaattactcaggtagaactggtcataatgtgcacctggcccctgccgttgcccccacttttccgggtgagcaactccttttctttagatccactatgcccggatgtagcgggtatcccaacatgaatttggattgcctactcccccaggaatgggtgctgcacttctaccaggaagcagctccagcacaatccgatgtggctctgctgagatttgtgaatccagacacaggtagggttctgtttgagtgcaagctccataaatcaggctatatcacagtggctcacaccggcccgtatgacttggttatcccccccaatggttattttagatttgattcctgggtcaaccagttctacacacttgcccccatgggaaatggaacggggcgcaggcgtgcattataa

SEQ ID NO: 26 - protein, 539 aa, synthetic construct
VKMASSDANPSDGSAANLVPEVNNEVMALEPVVGAAIAAPVAGQQNIIDPWIRNNFVQAPGGEFTVSPRNAPGEILWSAPLGPDLNPYLSHLSRMYNGYAGGFEVQVILAGNAFTAGKVIFAAVPPNFPTEGLSPSQVTMFPHIIVDVRQLEPVLIPLPDVRNNFYHYNQSHDSTLKLIAMLYTPLRANNAGDDVFTVSCRVLTRPSPDFDFIFLVPPTVESRTKPFTVPILTVEEMSNSRFPIPLEKLYTGPSSAFVVQPQNGRCTTDGVLLGTTQLSAVNICNFRGDVTHIVGSHDYTMNLASQNWSNYDPTEEIPAPLGTPDFVGKIQGLLTQTTRADGSTRAHKATVSTGSVHFTPKLGSVQFTTDTNNDFQTGQNTKFTPVGVIQDGDHHQNEPQQWVLPNYSGRTGHNVHLAPAVAPTFPGEQLLFFRSTMPGCSGYPNMNLDCLLPQEWVLHFYQEAAPAQSDVALLRFVNPDTGRVLFECKLHKSGYITVAHTGPYDLVIPPNGYFRFDSWVNQFYTLAPMGNGTGRRRAL

SEQ ID NO: 27 - DNA, 2626 bp, synthetic construct
  source 1..2626: mol_type = other DNA
gcggccgcccaccatggcttctttagataatttagtggcacgatatcagaggtgctttaatgaccagtctcttaaaaatagtactattgaacttgaaatacgttttcaacagataaattttttattattcaaaaccgtatatgaggcacttgtggcacaagagatccctagcaccatctcccacagcatccgctgcatcaaaaaagttcaccatgaaaaccactgccgggaaaaaattttgccgtcggaaaatctttacttcaaaaaacagcctctcatgttttttaagttttcagagcctgcatctctgggctgtaaggtctcgctggccatcgagcagcccattcgtaaatttatcttggactcctccgttctcgttcggctcaaaaatcgtacgacctttcgggtatctgaactttggaaaatagagcttaccattgtaaagcagctgatgggaagcgaggtctctgcaaaacttgccgctttcaaaacgcttctgtttgacaccccagagcaacaaacgacaaaaaatatgatgacgttaataaacccagatgacgaatatctttacgaaatagaaatagagtatacaggaaagcccgaatccctaacggcggcagatgttataaaaattaaaaacacggtgttgacacttatttctccgaaccatttaatgctaacagcctaccaccaggccattgaattcattgcctcccatatactgtcctcagaaatccttcttgctcgtattaagagcgggaagtgggggcttaaacgcctcctcccccaggtgaaatccatgaccaaagcggattacatgaaattttatccgcccgttggctactatgtaacggacaaagcagatggaattagaggcatcgccgtcattcaggacacgcaaatttatgtggttgcagaccagctatacagcctaggtaccaccggcattgaaccccttaaaccaaccattttggatggtgaatttatgcctgaaaaaaaagaattttatgggtttgacgtcatcatgtatgagggcaatctgttgacgcaacaggggtttgaaacaagaattgagtctttaagcaagggcattaaagtcttacaagcgtttaacataaaagcagaaatgaagccctttatttcgctaacaagtgcagatcccaacgtgctcctcaaaaactttgaaagcatttttaagaaaaaaactcgcccatattctattgatggcatcattttagtagaacctggcaattcttatctaaatacaaatacttttaagtggaagcccacctgggataacacattagactttttggtgcgaaaatgtccggagagtttaaacgtgccagagtacgcgcccaaaaaagggttttccctgcatctactatttgtaggcatctccggagagctttttaaaaaattagcgctaaattggtgtccaggatatacgaaactattccccgttacacagcgcaaccaaaactactttccagtacagtttcagccatcggattttccactggcatttctttattaccacccagatacctcgtcattttctaatatagatggaaaggtccttgaaatgcgttgtcttaagagagaaattaattacgtcagatgggaaattgtaaaaatccgggaggataggcagcaggatcttaaaaccggcgggtattttggcaatgatttcaaaacagccgaactcacatggcttaactatatggatcccttttcctttgaggaactggcaaagggcccttctggaatgtacttcgccggtgccaaaaccggcatataccgcgctcaaacagcacttatttcctttattaaacaagaaatcatccaaaaaataagtcaccaatcctgggttatcgatcttggaataggaaaagggcaggacctaggacgttacctggacgcaggggtaaggcatcttgttggaatcgataaggaccaaaccgcgctcgcggagcttgtttatcgaaaattttcgcatgctacaacccgacagcacaagcacgctaccaacatttacgtgttgcatcaagacctcgcagagcctgcgaaggaaatcagcgaaaaggtacaccaaatttacgggtttcccaaggagggagcttcttccattgttagcaacctgtttattcactatcttatgaaaaacacgcagcaggtggaaaacctggccgttctgtgccataagcttcttcagccggggggaatggtgtggtttaccaccatgttgggagaacaggtcttagaattacttcatgaaaatagaatagagctcaatgaagtatgggaggcacgtgaaaacgaggtggtcaaatttgctattaaacgtctctttaaagaggatatattacaggaaactgggcaagaaattggagtcctgttacccttcagcaatggcgacttctacaatgaatatcttgtgaacacagcgtttttaattaaaatatttaaacatcacggcttttccctagttcaaaagcagtcctttaaggactggattccagaatttcaaaactttagtaaaagtttgtataaaattcttacagaagccgataaaacttggacaagcctttttgggtttatttgtctgcgcaaaaattaacgatcg

SEQ ID NO: 28 - protein, 6 aa, synthetic construct
LVPRGS

SEQ ID NO: 29 - protein, 19 aa, synthetic construct
ATNFSLLKQAGDVEENPGP

SEQ ID NO: 30 - DNA, 18 bp, synthetic construct
  source 1..18: mol_type = other DNA
taatacgactcactatag

SEQ ID NO: 31 - DNA, 23 bp, synthetic construct
  source 1..23: mol_type = other DNA
aattaaccctcactaaagggaga

SEQ ID NO: 32 - DNA, 36 bp, synthetic construct
  source 1..36: mol_type = other DNA
ccattttgatacatgttgaacaatcaaatacagtgt

SEQ ID NO: 33 - DNA, 23 bp, synthetic construct
  source 1..23: mol_type = other DNA
gctagccttgtcatcgtcatcct

SEQ ID NO: 34 - DNA, 34 bp, synthetic construct
  source 1..34: mol_type = other DNA
gatgacaaggctagcactacccagctgtcagctg

SEQ ID NO: 35 - DNA, 42 bp, synthetic construct
  source 1..42: mol_type = other DNA
catgtatcaaaatggtcacaggtgcacattatgaccagttct

SEQ ID NO: 36 - DNA, 37 bp, synthetic construct
  source 1..37: mol_type = other DNA
gatgacaaggctagcaaaccattcaccgtcccaatct

SEQ ID NO: 37 - DNA, 36 bp, synthetic construct
  source 1..36: mol_type = other DNA
catgtatcaaaatggttataatgcacgcctgcgccc

SEQ ID NO: 38 - DNA, 36 bp, synthetic construct
  source 1..36: mol_type = other DNA
gatgacaaggctagcatgaagatggcgtcgagtgac

SEQ ID NO: 39 - DNA, 36 bp, synthetic construct; identical to SEQ ID NO: 37
  source 1..36: mol_type = other DNA

SEQ ID NO: 40 - DNA, 36 bp, synthetic construct; identical to SEQ ID NO: 32
  source 1..36: mol_type = other DNA

SEQ ID NO: 41 - DNA, 21 bp, synthetic construct
  source 1..21: mol_type = other DNA
aggaccggggttttcttccac

SEQ ID NO: 42 - DNA, 38 bp, synthetic construct
  source 1..38: mol_type = other DNA
gaaaaccccggtcctgctagcgtgaagatggcgtcgag

SEQ ID NO: 43 - DNA, 58 bp, synthetic construct
  source 1..58: mol_type = other DNA
catgtatcaaaatggttacttgtcatcgtcatccttgtaatctaatgcacgcctgcgc

SEQ ID NO: 44 - DNA, 38 bp, synthetic construct
  source 1..38: mol_type = other DNA
agaaaaccccggtcctgctagcaaaccattcaccgtcc

SEQ ID NO: 45 - DNA, 53 bp, synthetic construct
  source 1..53: mol_type = other DNA
catgtatcaaaatggttagtggtggtggtggtggtgtaatgcacgcctgcgcc

SEQ ID NO: 46 - DNA, 36 bp, synthetic construct
  source 1..36: mol_type = other DNA
gaaaaccccggtcctgtgaagatggcgtcgagtgac

SEQ ID NO: 47 - DNA, 67 bp, synthetic construct
  source 1..67: mol_type = other DNA
acatgtatcaaaatggttagtggtggtggtggtggtgcgagccacgggggaccaataatgcacgcct

SEQ ID NO: 48 - DNA, 36 bp, synthetic construct; identical to SEQ ID NO: 46
  source 1..36: mol_type = other DNA

SEQ ID NO: 49 - DNA, 48 bp, synthetic construct
  source 1..48: mol_type = other DNA
catgtatcaaaatggttagtggtggtggtggtggtgtaatgcacgcct

SEQ ID NO: 50 - DNA, 21 bp, synthetic construct
  source 1..21: mol_type = other DNA
ggcatttaatgcttttcagtg

SEQ ID NO: 51 - DNA, 21 bp, synthetic construct
  source 1..21: mol_type = other DNA
ggccacataacgcccctatag

SEQ ID NO: 52 - DNA, 24 bp, synthetic construct
  source 1..24: mol_type = other DNA
cattgcacgcttttgatgacttag

SEQ ID NO: 53 - DNA, 24 bp, synthetic construct
  source 1..24: mol_type = other DNA
cattgcacgcttttgatgacttag

SEQ ID NO: 54 - DNA, 36 bp, synthetic construct; identical to SEQ ID NO: 32
  source 1..36: mol_type = other DNA

SEQ ID NO: 55 - DNA, 23 bp, synthetic construct
  source 1..23: mol_type = other DNA
gctagccttgtcatcgtcatcct

SEQ ID NO: 56 - DNA, 34 bp, synthetic construct; identical to SEQ ID NO: 34
  source 1..34: mol_type = other DNA

SEQ ID NO: 57 - DNA, 42 bp, synthetic construct; identical to SEQ ID NO: 35
  source 1..42: mol_type = other DNA

SEQ ID NO: 58 - DNA, 37 bp, synthetic construct; identical to SEQ ID NO: 36
  source 1..37: mol_type = other DNA

SEQ ID NO: 59 - DNA, 36 bp, synthetic construct; identical to SEQ ID NO: 37
  source 1..36: mol_type = other DNA

SEQ ID NO: 60 - DNA, 36 bp, synthetic construct; identical to SEQ ID NO: 38
  source 1..36: mol_type = other DNA

SEQ ID NO: 61 - DNA, 36 bp, synthetic construct; identical to SEQ ID NO: 37
  source 1..36: mol_type = other DNA

SEQ ID NO: 62 - DNA, 36 bp, synthetic construct; identical to SEQ ID NO: 32
  source 1..36: mol_type = other DNA

SEQ ID NO: 63 - DNA, 21 bp, synthetic construct
  source 1..21: mol_type = other DNA
aggaccggggttttcttccac

SEQ ID NO: 64 - DNA, 38 bp, synthetic construct; identical to SEQ ID NO: 42
  source 1..38: mol_type = other DNA

SEQ ID NO: 65 - DNA, 58 bp, synthetic construct; identical to SEQ ID NO: 43
  source 1..58: mol_type = other DNA

SEQ ID NO: 66 - DNA, 38 bp, synthetic construct; identical to SEQ ID NO: 44
  source 1..38: mol_type = other DNA

SEQ ID NO: 67 - DNA, 53 bp, synthetic construct; identical to SEQ ID NO: 45
  source 1..53: mol_type = other DNA

SEQ ID NO: 68 - DNA, 36 bp, synthetic construct; identical to SEQ ID NO: 46
  source 1..36: mol_type = other DNA

SEQ ID NO: 69 - DNA, 67 bp, synthetic construct; identical to SEQ ID NO: 47
  source 1..67: mol_type = other DNA

SEQ ID NO: 70 - DNA, 36 bp, synthetic construct; identical to SEQ ID NO: 46
  source 1..36: mol_type = other DNA

SEQ ID NO: 71 - DNA, 48 bp, synthetic construct; identical to SEQ ID NO: 49
  source 1..48: mol_type = other DNA

SEQ ID NO: 72 - DNA, 36 bp, synthetic construct
  source 1..36: mol_type = other DNA
tgaccattttgatacatgttgaacaatcaaatacag

SEQ ID NO: 73 - DNA, 21 bp, synthetic construct
  source 1..21: mol_type = other DNA
aggaccggggttttcttccac

SEQ ID NO: 74 - DNA, 49 bp, synthetic construct
  source 1..49: mol_type = other DNA
gaaaaccccggtcctgtgtttgtttttcttgttttattgccactagtct

SEQ ID NO: 75 - DNA, 51 bp, synthetic construct
  source 1..51: mol_type = other DNA
gtatcaaaatggtcacttgtcatcgtcatccttgtaatcacgtgcccgccg

SEQ ID NO: 76 - DNA, 42 bp, synthetic construct
  source 1..42: mol_type = other DNA
accaccaccaccaccactgaccattttgatacatgttgaaca

SEQ ID NO: 77 - DNA, 34 bp, synthetic construct
  source 1..34: mol_type = other DNA
ggtggtggtggtggtgacgtgcccgccgaggaga

SEQ ID NO: 78 - protein, 712 aa, synthetic construct
PGPDYKDHDGDYKDHDIDYKDDDDKASVFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 79 - protein, 136 aa, synthetic construct
FNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQT

SEQ ID NO: 80 - protein, 539 aa, synthetic construct; identical to SEQ ID NO: 24

SEQ ID NO: 81 - protein, 318 aa, synthetic construct
SRTKPFTVPILTVEEMSNSRFPIPLEKLYTGPSSAFVVQPQNGRCTTDGVLLGTTQLSAVNICNFRGDVTHIVGSHDYTMNLASQNWSNYDPTEEIPAPLGTPDFVGKIQGLLTQTTRADGSTRAHKATVSTGSVHFTPKLGSVQFTTDTNNDFQTGQNTKFTPVGVIQDGDHHQNEPQQWVLPNYSGRTGHNVHLAPAVAPTFPGEQLLFFRSTMPGCSGYPNMNLDCLLPQEWVLHFYQEAAPAQSDVALLRFVNPDTGRVLFECKLHKSGYITVAHTGPYDLVIPPNGYFRFDSWVNQFYTLAPMGNGTGRRRAL

SEQ ID NO: 82 - protein, 143 aa, synthetic construct
TTQLSAVNICNFRGDVTHIVGSHDYTMNLASQNWSNYDPTEEIPAPLGTPDFVGKIQGLLTQTTRADGSTRAHKATVSTGSVHFTPKLGSVQFTTDTNNDFQTGQNTKFTPVGVIQDGDHHQNEPQQWVLPNYSGRTGHNVHL

SEQ ID NO: 83 - protein, 316 aa, synthetic construct
KPFTVPILTVEEMTNSRFPIPLEKLFTGPSGAFVVQPQNGRCTTDGVLLGTTQLSPVNICTFRGDVTHIAGTHNYTMNLASQNWNNYDPTEEIPAPLGTPDFVGRIQGMLTQTTRGDGSTRGHKATVSTGDVHFTPKLGSIQFNTDTNNDFETGQNTKFTPVGVVQDGNGTHQNEPQQWVLPSYSGRTGHNVHLAPAVAPTFPGEQLLFFRSTMPGCSGYPNMNLDCLLPQEWVQHFYQEAAPAQSDVALLRFVNPDTGRVLFECKLHKSGYVTVAHTGQHDLVIPPNGYFRFDSWVNQFYTLAPLGNGTGRRRAL

SEQ ID NO: 84 - protein, 540 aa, synthetic construct
MKMASKDANPSDGSTANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKVIFAAVPPNFPTEGLSPSQVTMFPHIVVDVRQLEPVLIPLPDVRNNFYHYNQSNDPTIKLIAMLYTPLRANNAGEDVFTVSCRVLTRPSPDFDFIFLVPPTVESRTKPFTVPILTVEEMTNSRFPIPLEKLFTGPSGAFVVQPQNGRCTTDGVLLGTTQLSPVNICTFRGDVTHIAGTHNYTMNLASQNWNNYDPTEEIPAPLGTPDFVGRIQGMLTQTTRGDGSTRGHKATVSTGDVHFTPKLGSIQFNTDTNNDFETGQNTKFTPVGVVQDGNGTHQNEPQQWVLPSYSGRTGHNVHLAPAVAPTFPGEQLLFFRSTMPGCSGYPNMNLDCLLPQEWVQHFYQEAAPAQSDVALLRFVNPDTGRVLFECKLHKSGYVTVAHTGQHDLVIPPNGYFRFDSWVNQFYTLAPLGNGTGRRRAL

(19) United States
(12) Patent Application Publication
(10) Pub. No.: US 2025/0263443 A1
(43) Pub. Date: Aug. 21, 2025

(54) RECOMBINANT ROTAVIRUS EXPRESSING EXOGENOUS PROTEIN AND USES THEREOF

(71) Applicant: THE TRUSTEES OF INDIANA UNIVERSITY, Bloomington, IN (US)

(72) Inventors: John Thomas PATTON, Bloomington, IN (US); Asha Ann Philip, Bloomington, IN (US)

(21) Appl. No.: 18/702,470

(22) PCT Filed: Oct. 18, 2022
(86) PCT No.: PCT/US2022/078305

§ 371 Date: Apr. 18, 2024

Related U.S. Application Data
(60) Provisional application No. 63/256,960, filed on Oct. 18, 2021.
(60) Provisional application No. 63/256,875, filed on Oct. 18, 2021.

(51) Int. Cl. C07K 14/005; A61K 39/00; C12N 15/86

(52) U.S. Cl. CPC C07K 14/005; C12N 15/86; A61K 2039/5254; C12N 2720/12322; C12N 2720/12334; C12N 2720/12343; C12N 2770/16022; C12N 2770/16034; C12N 2770/20022; C12N 2770/20034

(57) ABSTRACT

Disclosed herein are polynucleotides encoding recombinant rotaviruses (RVs), methods of using the same, and systems for generating the recombinant RVs.

### CROSS-REFERENCE TO RELATED APPLICATIONS

The present application claims the benefit of U.S. Provisional Application No. 63/256,960, filed Oct. 18, 2021, and U.S. Provisional Application No. 63/256,875, filed Oct. 18, 2021, each of which is incorporated by reference herein in its entirety.

### STATEMENT OF GOVERNMENTAL RIGHTS

This invention was made with government support under TR002529 and AI44881 awarded by the National Institutes of Health. The Government has certain rights in the invention.

### SEQUENCE LISTING

A Sequence Listing accompanies this application and is submitted as an xml file of the sequence listing in ST26 format named “144578_00351.xml” which is 120,469 bytes in size and was created on Oct. 18, 2022. The sequence listing is electronically submitted via EFS-Web with the application and is incorporated herein by reference in its entirety.

### BACKGROUND

Rotavirus (RV) and norovirus (NoV) are the leading cause of acute gastroenteritis (AGE) and acute diarrheal episodes in young children and the elderly (1, 2). The development and introduction of effective RV vaccines—a monovalent vaccine (RV1), Rotarix (GSK Biologicals) and a pentavalent vaccine (RV5), RotaTeq (Merck and Company), in the childhood immunization programs in the US and many other countries resulted in reducing the incidence of RV hospitalizations and mortality, and these vaccines are quite successful in generating neutralizing antibodies in vaccinated children (3, 4). In countries where RV vaccines are largely used, an increase of NoV mediated diarrheal diseases and hospitalizations in children during the first 5 years of life became more evident (5, 6). The incidence of NoV disease is higher in young children, in the elderly population, and in subjects with lower immune defenses, for whom effective preventive measures are needed urgently (7). NoV disease is extremely contagious, only 10 infectious particles are required to cause AGE, and they have high environmental stability, and shedding after infection lasts for weeks (8). However, it has been very difficult to develop effective anti-NoV drugs or vaccines for many years due to the lack of adequate cell lines for viral culture and successful animal models for drug and vaccine evaluation (9, 10). Therefore, there is a need in the art for novel compositions and methods to elicit immunity to NoV.

### SUMMARY

In an aspect of the current disclosure, polynucleotides are provided. In some embodiments, the polynucleotides comprise: a sequence encoding a rotavirus (RV) NSP3 protein; and a heterologous polynucleotide. In some embodiments, the polynucleotide is operably linked to a promoter. In some embodiments, the promoter is a T7 promoter or a T3 promoter. In some embodiments, the promoter is a T7 promoter. In some embodiments, the polynucleotide encodes a positive sense viral transcript. In some embodiments, the sequence encoding an RV NSP3 is a sequence encoding SEQ ID NO: 1, or a sequence with at least about 90% identity to SEQ ID NO: 1. In some embodiments, the heterologous polynucleotide encodes a peptide or protein. In some embodiments, the heterologous polynucleotide encodes a peptide or protein and is in frame with the NSP3. In some embodiments, the peptide or protein comprises an antigenic peptide or protein. In some embodiments, the peptide or protein comprises a microorganismal peptide or protein. In some embodiments, the peptide or protein comprises a bacterial peptide or protein. In some embodiments, the peptide or protein comprises a viral peptide or protein. In some embodiments, the peptide or protein comprises a norovirus (NoV) peptide or protein. In some embodiments, the NoV peptide or protein comprises NoV VP1 protein. In some embodiments, the NoV peptide or protein is selected from SEQ ID NOs: 24, 26, or 80-84. In some embodiments, the peptide or protein comprises a SARS-CoV-2 protein or peptide. In some embodiments, the SARS-CoV-2 protein or peptide is selected from an N protein, an S protein, or fragment of either an N protein or an S protein. In some embodiments, the SARS-CoV-2 protein is an S1 protein or a fragment thereof. In some embodiments, the peptide or protein comprises a cleavage site. In some embodiments, the cleavage site is a protease cleavage site. In some embodiments, the cleavage site is a thrombin cleavage site (SEQ ID NO: 28). In some embodiments, the cleavage site is a self-cleaving peptide sequence. In some embodiments, the self-cleaving peptide sequence is porcine teschovirus 2A element (SEQ ID NO: 29). In some embodiments, the peptide or protein comprises a linker. In some embodiments, the linker is a GAG flexible linker or a GSG flexible linker. In some embodiments, the heterologous polynucleotide is about 2.6 kb or less in length. In some embodiments, the heterologous polynucleotide is about 1.55 kb or less in length. In some embodiments, the heterologous polynucleotide is about 1.3 kb or less in length. In some embodiments, the peptide or protein is a glycoprotein. In some embodiments, the peptide or protein comprises one or more glycosylation site. In some embodiments, the heterologous polynucleotide encodes a reporter. In some embodiments, the reporter is selected from a fluorescent reporter, an enzyme, and an antigen tag. In some embodiments, the reporter is fused in-frame 3′ to the sequence encoding the RV protein. In some embodiments, the polynucleotides encoding a protein selected from SEQ ID NOs: 2-11, or a sequence with at least about 90% identity to a sequence selected from SEQ ID NOs: 2-11. In some embodiments, the polynucleotides comprise any one of SEQ ID NOs: 13-22 or a sequence with at least about 90% identity to any one of SEQ ID NOs: 13-22.

In another aspect of the current disclosure, infectious particles are provided. In some embodiments, the infectious particles comprise a polynucleotide comprising a sequence encoding a rotavirus (RV) NSP3 protein; and a heterologous polynucleotide. In some embodiments, the polynucleotide is operably linked to a promoter. In some embodiments, the promoter is a T7 promoter or a T3 promoter. In some embodiments, the promoter is a T7 promoter. In some embodiments, the polynucleotide encodes a positive sense viral transcript. In some embodiments, the sequence encoding an RV NSP3 is a sequence encoding SEQ ID NO: 1, or a sequence with at least about 90% identity to SEQ ID NO: 1. In some embodiments, the heterologous polynucleotide encodes a peptide or protein. In some embodiments, the heterologous polynucleotide encodes a peptide or protein and is in frame with the NSP3. In some embodiments, the peptide or protein comprises an antigenic peptide or protein. In some embodiments, the peptide or protein comprises a microorganismal peptide or protein. In some embodiments, the peptide or protein comprises a bacterial peptide or protein. In some embodiments, the peptide or protein comprises a viral peptide or protein. In some embodiments, the peptide or protein comprises a norovirus (NoV) peptide or protein. In some embodiments, the NoV peptide or protein comprises NoV VP1 protein. In some embodiments, the NoV peptide or protein is selected from SEQ ID NOs: 24, 26, or 80-84. In some embodiments, the peptide or protein comprises a SARS-CoV-2 protein or peptide. In some embodiments, the SARS-CoV-2 protein or peptide is selected from an N protein, an S protein, or fragment of either an N protein or an S protein. In some embodiments, the SARS-CoV-2 protein is an S1 protein or a fragment thereof. In some embodiments, the peptide or protein comprises a cleavage site. In some embodiments, the cleavage site is a protease cleavage site. In some embodiments, the cleavage site is a thrombin cleavage site (SEQ ID NO: 28). In some embodiments, the cleavage site is a self-cleaving peptide sequence. In some embodiments, the self-cleaving peptide sequence is porcine teschovirus 2A element (SEQ ID NO: 29). In some embodiments, the peptide or protein comprises a linker. In some embodiments, the linker is a GAG flexible linker or a GSG flexible linker. In some embodiments, the heterologous polynucleotide is about 2.6 kb or less in length. In some embodiments, the heterologous polynucleotide is about 1.55 kb or less in length. In some embodiments, the heterologous polynucleotide is about 1.3 kb or less in length. In some embodiments, the peptide or protein is a glycoprotein. In some embodiments, the peptide or protein comprises one or more glycosylation site. In some embodiments, the heterologous polynucleotide encodes a reporter. In some embodiments, the reporter is selected from a fluorescent reporter, an enzyme, and an antigen tag. In some embodiments, the reporter is fused in-frame 3′ to the sequence encoding the RV protein. In some embodiments, the polynucleotides encoding a protein selected from SEQ ID NOs: 2-11, or a sequence with at least about 90% identity to a sequence selected from SEQ ID NOs: 2-11. In some embodiments, the polynucleotides comprise any one of SEQ ID NOs: 13-22 or a sequence with at least about 90% identity to any one of SEQ ID NOs: 13-22.

In some embodiments, the infectious particles are made by introducing a polynucleotide comprising a sequence encoding a rotavirus (RV) NSP3 protein; and a heterologous polynucleotide into a cell. In some embodiments, the polynucleotide is operably linked to a promoter. In some embodiments, the promoter is a T7 promoter or a T3 promoter. In some embodiments, the promoter is a T7 promoter. In some embodiments, the polynucleotide encodes a positive sense viral transcript. In some embodiments, the sequence encoding an RV NSP3 is a sequence encoding SEQ ID NO: 1, or a sequence with at least about 90% identity to SEQ ID NO: 1. In some embodiments, the heterologous polynucleotide encodes a peptide or protein. In some embodiments, the heterologous polynucleotide encodes a peptide or protein and is in frame with the NSP3. In some embodiments, the peptide or protein comprises an antigenic peptide or protein. In some embodiments, the peptide or protein comprises a microorganismal peptide or protein. In some embodiments, the peptide or protein comprises a bacterial peptide or protein. In some embodiments, the peptide or protein comprises a viral peptide or protein. In some embodiments, the peptide or protein comprises a norovirus (NoV) peptide or protein. In some embodiments, the NoV peptide or protein comprises NoV VP1 protein. In some embodiments, the NoV peptide or protein is selected from SEQ ID NOs: 24, 26, or 80-84. In some embodiments, the peptide or protein comprises a SARS-CoV-2 protein or peptide. In some embodiments, the SARS-CoV-2 protein or peptide is selected from an N protein, an S protein, or fragment of either an N protein or an S protein. In some embodiments, the SARS-CoV-2 protein is an S1 protein or a fragment thereof. In some embodiments, the peptide or protein comprises a cleavage site. In some embodiments, the cleavage site is a protease cleavage site. In some embodiments, the cleavage site is a thrombin cleavage site (SEQ ID NO: 28). In some embodiments, the cleavage site is a self-cleaving peptide sequence. In some embodiments, the self-cleaving peptide sequence is porcine teschovirus 2A element (SEQ ID NO: 29). In some embodiments, the peptide or protein comprises a linker. In some embodiments, the linker is a GAG flexible linker or a GSG flexible linker. In some embodiments, the heterologous polynucleotide is about 2.6 kb or less in length. In some embodiments, the heterologous polynucleotide is about 1.55 kb or less in length. In some embodiments, the heterologous polynucleotide is about 1.3 kb or less in length. In some embodiments, the peptide or protein is a glycoprotein. In some embodiments, the peptide or protein comprises one or more glycosylation site. In some embodiments, the heterologous polynucleotide encodes a reporter. In some embodiments, the reporter is selected from a fluorescent reporter, an enzyme, and an antigen tag. In some embodiments, the reporter is fused in-frame 3′ to the sequence encoding the RV protein. In some embodiments, the polynucleotides encoding a protein selected from SEQ ID NOs: 2-11, or a sequence with at least about 90% identity to a sequence selected from SEQ ID NOs: 2-11. In some embodiments, the polynucleotides comprise any one of SEQ ID NOs: 13-22 or a sequence with at least about 90% identity to any one of SEQ ID NOs: 13-22.

In another aspect of the current disclosure, pharmaceutical composition comprising an infectious particle comprising a polynucleotide comprising a sequence encoding a rotavirus (RV) NSP3 protein; and a heterologous polynucleotide, optionally, further comprising a pharmaceutically acceptable carrier or excipient. In some embodiments, the polynucleotide is operably linked to a promoter. In some embodiments, the promoter is a T7 promoter or a T3 promoter. In some embodiments, the promoter is a T7 promoter. In some embodiments, the polynucleotide encodes a positive sense viral transcript. In some embodiments, the sequence encoding an RV NSP3 is a sequence encoding SEQ ID NO: 1, or a sequence with at least about 90% identity to SEQ ID NO: 1. In some embodiments, the heterologous polynucleotide encodes a peptide or protein. In some embodiments, the heterologous polynucleotide encodes a peptide or protein and is in frame with the NSP3. In some embodiments, the peptide or protein comprises an antigenic peptide or protein. In some embodiments, the peptide or protein comprises a microorganismal peptide or protein. In some embodiments, the peptide or protein comprises a bacterial peptide or protein. In some embodiments, the peptide or protein comprises a viral peptide or protein. In some embodiments, the peptide or protein comprises a norovirus (NoV) peptide or protein. In some embodiments, the NoV peptide or protein comprises NoV VP1 protein. In some embodiments, the NoV peptide or protein is selected from SEQ ID NOs: 24, 26, or 80-84. In some embodiments, the peptide or protein comprises a SARS-CoV-2 protein or peptide. In some embodiments, the SARS-CoV-2 protein or peptide is selected from an N protein, an S protein, or fragment of either an N protein or an S protein. In some embodiments, the SARS-CoV-2 protein is an S1 protein or a fragment thereof. In some embodiments, the peptide or protein comprises a cleavage site. In some embodiments, the cleavage site is a protease cleavage site. In some embodiments, the cleavage site is a thrombin cleavage site (SEQ ID NO: 28). In some embodiments, the cleavage site is a self-cleaving peptide sequence. In some embodiments, the self-cleaving peptide sequence is porcine teschovirus 2A element (SEQ ID NO: 29). In some embodiments, the peptide or protein comprises a linker. In some embodiments, the linker is a GAG flexible linker or a GSG flexible linker. In some embodiments, the heterologous polynucleotide is about 2.6 kb or less in length. In some embodiments, the heterologous polynucleotide is about 1.55 kb or less in length. In some embodiments, the heterologous polynucleotide is about 1.3 kb or less in length. In some embodiments, the peptide or protein is a glycoprotein. In some embodiments, the peptide or protein comprises one or more glycosylation site. In some embodiments, the heterologous polynucleotide encodes a reporter. In some embodiments, the reporter is selected from a fluorescent reporter, an enzyme, and an antigen tag. In some embodiments, the reporter is fused in-frame 3′ to the sequence encoding the RV protein. In some embodiments, the polynucleotides encoding a protein selected from SEQ ID NOs: 2-11, or a sequence with at least about 90% identity to a sequence selected from SEQ ID NOs: 2-11. In some embodiments, the polynucleotides comprise any one of SEQ ID NOs: 13-22 or a sequence with at least about 90% identity to any one of SEQ ID NOs: 13-22.

In some embodiments, the pharmaceutical compositions comprise an infectious particle made by introducing a polynucleotide comprising a sequence encoding a rotavirus (RV) NSP3 protein; and a heterologous polynucleotide into a cell. In some embodiments, the polynucleotide is operably linked to a promoter. In some embodiments, the promoter is a T7 promoter or a T3 promoter. In some embodiments, the promoter is a T7 promoter. In some embodiments, the polynucleotide encodes a positive sense viral transcript. In some embodiments, the sequence encoding an RV NSP3 is a sequence encoding SEQ ID NO: 1, or a sequence with at least about 90% identity to SEQ ID NO: 1. In some embodiments, the heterologous polynucleotide encodes a peptide or protein. In some embodiments, the heterologous polynucleotide encodes a peptide or protein and is in frame with the NSP3. In some embodiments, the peptide or protein comprises an antigenic peptide or protein. In some embodiments, the peptide or protein comprises a microorganismal peptide or protein. In some embodiments, the peptide or protein comprises a bacterial peptide or protein. In some embodiments, the peptide or protein comprises a viral peptide or protein. In some embodiments, the peptide or protein comprises a norovirus (NoV) peptide or protein. In some embodiments, the NoV peptide or protein comprises NoV VP1 protein. In some embodiments, the NoV peptide or protein is selected from SEQ ID NOs: 24, 26, or 80-84. In some embodiments, the peptide or protein comprises a SARS-CoV-2 protein or peptide. In some embodiments, the SARS-CoV-2 protein or peptide is selected from an N protein, an S protein, or fragment of either an N protein or an S protein. In some embodiments, the SARS-CoV-2 protein is an S1 protein or a fragment thereof. In some embodiments, the peptide or protein comprises a cleavage site. In some embodiments, the cleavage site is a protease cleavage site. In some embodiments, the cleavage site is a thrombin cleavage site (SEQ ID NO: 28). In some embodiments, the cleavage site is a self-cleaving peptide sequence. In some embodiments, the self-cleaving peptide sequence is porcine teschovirus 2A element (SEQ ID NO: 29). In some embodiments, the peptide or protein comprises a linker. In some embodiments, the linker is a GAG flexible linker or a GSG flexible linker. In some embodiments, the heterologous polynucleotide is about 2.6 kb or less in length. In some embodiments, the heterologous polynucleotide is about 1.55 kb or less in length. In some embodiments, the heterologous polynucleotide is about 1.3 kb or less in length. In some embodiments, the peptide or protein is a glycoprotein. In some embodiments, the peptide or protein comprises one or more glycosylation site. In some embodiments, the heterologous polynucleotide encodes a reporter. In some embodiments, the reporter is selected from a fluorescent reporter, an enzyme, and an antigen tag. In some embodiments, the reporter is fused in-frame 3′ to the sequence encoding the RV protein. In some embodiments, the polynucleotides encoding a protein selected from SEQ ID NOs: 2-11, or a sequence with at least about 90% identity to a sequence selected from SEQ ID NOs: 2-11. In some embodiments, the polynucleotides comprise any one of SEQ ID NOs: 13-22 or a sequence with at least about 90% identity to any one of SEQ ID NOs: 13-22. In some embodiments, the pharmaceutical compositions further comprise a pharmaceutically acceptable carrier.

In another aspect of the current disclosure, methods of eliciting an immune response to one or more microorganism in a subject are provided. In some embodiments, the methods comprise: administering an effective amount of a pharmaceutical composition comprising an infectious particle comprising a polynucleotide comprising a sequence encoding a rotavirus (RV) NSP3 protein; and a heterologous polynucleotide, optionally, further comprising a pharmaceutically acceptable carrier or excipient; to a subject to elicit an immune response to the one or more microorganism. In some embodiments, the pharmaceutical compositions comprise an infectious particle made by introducing a polynucleotide comprising a sequence encoding a rotavirus (RV) NSP3 protein; and a heterologous polynucleotide into a cell. In some embodiments, the polynucleotide is operably linked to a promoter. In some embodiments, the promoter is a T7 promoter or a T3 promoter. In some embodiments, the promoter is a T7 promoter. In some embodiments, the polynucleotide encodes a positive sense viral transcript. In some embodiments, the sequence encoding an RV NSP3 is a sequence encoding SEQ ID NO: 1, or a sequence with at least about 90% identity to SEQ ID NO: 1. In some embodiments, the heterologous polynucleotide encodes a peptide or protein. In some embodiments, the heterologous polynucleotide encodes a peptide or protein and is in frame with the NSP3. In some embodiments, the peptide or protein comprises an antigenic peptide or protein. In some embodiments, the peptide or protein comprises a microorganismal peptide or protein. In some embodiments, the peptide or protein comprises a bacterial peptide or protein. In some embodiments, the peptide or protein comprises a viral peptide or protein. In some embodiments, the peptide or protein comprises a norovirus (NoV) peptide or protein. In some embodiments, the NoV peptide or protein comprises NoV VP1 protein. In some embodiments, the NoV peptide or protein is selected from SEQ ID NOs: 24, 26, or 80-84. In some embodiments, the peptide or protein comprises a SARS-CoV-2 protein or peptide. In some embodiments, the SARS-CoV-2 protein or peptide is selected from an N protein, an S protein, or fragment of either an N protein or an S protein. In some embodiments, the SARS-CoV-2 protein is an S1 protein or a fragment thereof. In some embodiments, the peptide or protein comprises a cleavage site. In some embodiments, the cleavage site is a protease cleavage site. In some embodiments, the cleavage site is a thrombin cleavage site (SEQ ID NO: 28). In some embodiments, the cleavage site is a self-cleaving peptide sequence. In some embodiments, the self-cleaving peptide sequence is porcine teschovirus 2A element (SEQ ID NO: 29). In some embodiments, the peptide or protein comprises a linker. In some embodiments, the linker is a GAG flexible linker or a GSG flexible linker. In some embodiments, the heterologous polynucleotide is about 2.6 kb or less in length. In some embodiments, the heterologous polynucleotide is about 1.55 kb or less in length. In some embodiments, the heterologous polynucleotide is about 1.3 kb or less in length. In some embodiments, the peptide or protein is a glycoprotein. In some embodiments, the peptide or protein comprises one or more glycosylation site. In some embodiments, the heterologous polynucleotide encodes a reporter. In some embodiments, the reporter is selected from a fluorescent reporter, an enzyme, and an antigen tag. In some embodiments, the reporter is fused in-frame 3′ to the sequence encoding the RV protein. In some embodiments, the polynucleotides encoding a protein selected from SEQ ID NOs: 2-11, or a sequence with at least about 90% identity to a sequence selected from SEQ ID NOs: 2-11. In some embodiments, the polynucleotides comprise any one of SEQ ID NOs: 13-22 or a sequence with at least about 90% identity to any one of SEQ ID NOs: 13-22. In some embodiments, the pharmaceutical compositions further comprise a pharmaceutically acceptable carrier. In some embodiments, the one or more pathogens comprises NoV. In some embodiments, the one or more pathogens comprises RV and NoV. In some embodiments, the one or more pathogens comprises SARS-CoV-2. In some embodiments, the one or more pathogens comprises RV and SARS-CoV-2.

In another aspect of the current disclosure, methods of vaccinating a subject against one or more pathogens are provided. In some embodiments, the methods comprise: administering an effective amount of a pharmaceutical composition comprising an infectious particle comprising a polynucleotide comprising a sequence encoding a rotavirus (RV) NSP3 protein; and a heterologous polynucleotide, optionally, further comprising a pharmaceutically acceptable carrier or excipient to a subject to vaccinate a subject against the one or more pathogens. In some embodiments, the pharmaceutical compositions comprise an infectious particle made by introducing a polynucleotide comprising a sequence encoding a rotavirus (RV) NSP3 protein; and a heterologous polynucleotide into a cell. In some embodiments, the polynucleotide is operably linked to a promoter. In some embodiments, the promoter is a T7 promoter or a T3 promoter. In some embodiments, the promoter is a T7 promoter. In some embodiments, the polynucleotide encodes a positive sense viral transcript. In some embodiments, the sequence encoding an RV NSP3 is a sequence encoding SEQ ID NO: 1, or a sequence with at least about 90% identity to SEQ ID NO: 1. In some embodiments, the heterologous polynucleotide encodes a peptide or protein. In some embodiments, the heterologous polynucleotide encodes a peptide or protein and is in frame with the NSP3. In some embodiments, the peptide or protein comprises an antigenic peptide or protein. In some embodiments, the peptide or protein comprises a microorganismal peptide or protein. In some embodiments, the peptide or protein comprises a bacterial peptide or protein. In some embodiments, the peptide or protein comprises a viral peptide or protein. In some embodiments, the peptide or protein comprises a norovirus (NoV) peptide or protein. In some embodiments, the NoV peptide or protein comprises NoV VP1 protein. In some embodiments, the NoV peptide or protein is selected from SEQ ID NOs: 24, 26, or 80-84. In some embodiments, the peptide or protein comprises a SARS-CoV-2 protein or peptide. In some embodiments, the SARS-CoV-2 protein or peptide is selected from an N protein, an S protein, or fragment of either an N protein or an S protein. In some embodiments, the SARS-CoV-2 protein is an S1 protein or a fragment thereof. In some embodiments, the peptide or protein comprises a cleavage site. In some embodiments, the cleavage site is a protease cleavage site. In some embodiments, the cleavage site is a thrombin cleavage site (SEQ ID NO: 28). In some embodiments, the cleavage site is a self-cleaving peptide sequence. In some embodiments, the self-cleaving peptide sequence is porcine teschovirus 2A element (SEQ ID NO: 29). In some embodiments, the peptide or protein comprises a linker. In some embodiments, the linker is a GAG flexible linker or a GSG flexible linker. In some embodiments, the heterologous polynucleotide is about 2.6 kb or less in length. In some embodiments, the heterologous polynucleotide is about 1.55 kb or less in length. In some embodiments, the heterologous polynucleotide is about 1.3 kb or less in length. In some embodiments, the peptide or protein is a glycoprotein. In some embodiments, the peptide or protein comprises one or more glycosylation site. In some embodiments, the heterologous polynucleotide encodes a reporter. In some embodiments, the reporter is selected from a fluorescent reporter, an enzyme, and an antigen tag. In some embodiments, the reporter is fused in-frame 3′ to the sequence encoding the RV protein. In some embodiments, the polynucleotides encoding a protein selected from SEQ ID NOs: 2-11, or a sequence with at least about 90% identity to a sequence selected from SEQ ID NOs: 2-11. In some embodiments, the polynucleotides comprise any one of SEQ ID NOs: 13-22 or a sequence with at least about 90% identity to any one of SEQ ID NOs: 13-22. In some embodiments, the pharmaceutical compositions further comprise a pharmaceutically acceptable carrier. In some embodiments, the one or more pathogens comprises NoV. In some embodiments, the one or more pathogens comprises RV and NoV. In some embodiments, the one or more pathogens comprises SARS-CoV-2. In some embodiments, the one or more pathogens comprises RV and SARS-CoV-2. In some embodiments, the one or more pathogens comprises NoV. In some embodiments, the one or more pathogens comprises RV and NoV. In some embodiments, the one or more pathogens comprises SARS-CoV-2. In some embodiments, the one or more pathogens comprises RV and SARS-CoV-2.

In another aspect of the current disclosure, methods of generating recombinant rotavirus (RV) in vitro are provided. In some embodiments, the methods comprise: introducing a polynucleotide comprising a sequence encoding a rotavirus (RV) NSP3 protein; and a heterologous polynucleotide into a cell; allowing the cell to express the polynucleotide; incubating the cells for a sufficient time to produce RV; and harvesting virus produced by the cells to generate RV in vitro. In some embodiments, the polynucleotide is operably linked to a promoter. In some embodiments, the promoter is a T7 promoter or a T3 promoter. In some embodiments, the promoter is a T7 promoter. In some embodiments, the polynucleotide encodes a positive sense viral transcript. In some embodiments, the sequence encoding an RV NSP3 is a sequence encoding SEQ ID NO: 1, or a sequence with at least about 90% identity to SEQ ID NO: 1. In some embodiments, the heterologous polynucleotide encodes a peptide or protein. In some embodiments, the heterologous polynucleotide encodes a peptide or protein and is in frame with the NSP3. In some embodiments, the peptide or protein comprises an antigenic peptide or protein. In some embodiments, the peptide or protein comprises a microorganismal peptide or protein. In some embodiments, the peptide or protein comprises a bacterial peptide or protein. In some embodiments, the peptide or protein comprises a viral peptide or protein. In some embodiments, the peptide or protein comprises a norovirus (NoV) peptide or protein. In some embodiments, the NoV peptide or protein comprises NoV VP1 protein. In some embodiments, the NoV peptide or protein is selected from SEQ ID NOs: 24, 26, or 80-84. In some embodiments, the peptide or protein comprises a SARS-CoV-2 protein or peptide. In some embodiments, the SARS-CoV-2 protein or peptide is selected from an N protein, an S protein, or fragment of either an N protein or an S protein. In some embodiments, the SARS-CoV-2 protein is an S1 protein or a fragment thereof. In some embodiments, the peptide or protein comprises a cleavage site. In some embodiments, the cleavage site is a protease cleavage site. In some embodiments, the cleavage site is a thrombin cleavage site (SEQ ID NO: 28). In some embodiments, the cleavage site is a self-cleaving peptide sequence. In some embodiments, the self-cleaving peptide sequence is porcine teschovirus 2A element (SEQ ID NO: 29). In some embodiments, the peptide or protein comprises a linker. In some embodiments, the linker is a GAG flexible linker or a GSG flexible linker. In some embodiments, the heterologous polynucleotide is about 2.6 kb or less in length. In some embodiments, the heterologous polynucleotide is about 1.55 kb or less in length. In some embodiments, the heterologous polynucleotide is about 1.3 kb or less in length. In some embodiments, the peptide or protein is a glycoprotein. In some embodiments, the peptide or protein comprises one or more glycosylation site. In some embodiments, the heterologous polynucleotide encodes a reporter. In some embodiments, the reporter is selected from a fluorescent reporter, an enzyme, and an antigen tag. In some embodiments, the reporter is fused in-frame 3′ to the sequence encoding the RV protein. In some embodiments, the polynucleotides encoding a protein selected from SEQ ID NOs: 2-11, or a sequence with at least about 90% identity to a sequence selected from SEQ ID NOs: 2-11. In some embodiments, the polynucleotides comprise any one of SEQ ID NOs: 13-22 or a sequence with at least about 90% identity to any one of SEQ ID NOs: 13-22. In some embodiments, the method further comprises introducing one or more additional polynucleotides into the cell before the allowing step, wherein the one or more additional polynucleotides comprise a sequence encoding an RV protein selected from VP1, VP2, VP3, VP4, VP6, VP7, NSP1, NSP2, NSP3, NSP4, and NSP5, wherein each sequence encoding an RV protein is operably linked to a promoter. In some embodiments, the one or more additional polynucleotides comprise a sequence encoding an RV protein selected from VP1, VP2, VP3, VP4, VP6, VP7, NSP1, NSP2, NSP4, and NSP5. In some embodiments, the one or more additional polynucleotides comprise a sequence encoding a capping enzyme operably linked to a promoter. In some embodiments, the capping enzyme is African swine fever virus capping enzyme. In some embodiments, the cell is selected from an MA-104 cell, a Vero cell, and a BHK-1 cell. In some embodiments, the cell expresses a heterologous RNA polymerase. In some embodiments, the heterologous RNA polymerase is selected from T7 RNA polymerase and T3 RNA polymerase. In some embodiments, the cell is a BHK-1 cell comprising T7 RNA polymerase. In some embodiments, the method produces an RV protein fused to a glycoprotein when expressed in the cell.

In another aspect of the current disclosure, cells are provided. In some embodiments, the cells comprise a polynucleotide comprising a sequence encoding a rotavirus (RV) NSP3 protein; and a heterologous polynucleotide. In some embodiments, the cells comprise a infectious particle comprising a polynucleotide comprising a sequence encoding a rotavirus (RV) NSP3 protein; and a heterologous polynucleotide. In some embodiments, the cells comprise an infectious particle made by introducing a polynucleotide comprising a polynucleotide comprising a sequence encoding a rotavirus (RV) NSP3 protein; and a heterologous polynucleotide into a cell. In some embodiments, the cell is an MA-104 cell, a Vero cell or a BHK-1 cell. In some embodiments, the cell expresses a heterologous RNA polymerase. In some embodiments, the heterologous RNA polymerase is selected from T7 RNA polymerase and T3 RNA polymerase.

In another aspect of the current disclosure, systems, platforms, or kits for generating recombinant rotavirus (RV) are provided. In some embodiments, the systems, platforms, or kits comprise: (a) a polynucleotide comprising a sequence encoding a rotavirus (RV) NSP3 protein; and a heterologous polynucleotide; and (b) cells capable of expressing the polynucleotides of (a). In some embodiments, the systems, platforms, or kits further comprise one or more additional polynucleotides comprising a sequence encoding an RV protein selected from VP1, VP2, VP3, VP4, VP6, VP7, NSP1, NSP2, NSP3, NSP4, and NSP5, wherein each sequence encoding an RV protein is operably linked to a promoter. In some embodiments, the one or more additional polynucleotides comprise at least one sequence encoding an RV protein selected from VP1, VP2, VP3, VP4, VP6, VP7, NSP1, NSP2, NSP4, and NSP5. In some embodiments, the cells comprise a heterologous RNA polymerase and, optionally, comprising African swine fever virus capping enzyme. In some embodiments, the cells comprise a cells from a cell line selected from MA-104 cells, Vero cells, and BHK-1 cells. In some embodiments, the cells comprise BHK-1 cells comprising T7 RNA polymerase. In some embodiments, the cells comprise BHK-1 cells comprising T7 RNA polymerase, Vero cells, and MA-104 cells.

### BRIEF DESCRIPTION OF THE FIGURES

FIG. 1A. Domains of the human NoV VP1 capsid protein. VP1 can be subdivided into shell(S) and protruding (P) domains. The P domain is further resolved into P1 and P2 subdomains.

FIG. 1B. Surface representation of NoV capsid with the S domain (green) and P1 (cyan) and P2 (blue) subdomains of VP1 distinguished by color.

FIG. 1C. Ribbon representation of a NoV VP1 dimer: S (green), P1 (cyan) and P2 (blue).

FIG. 2. Plasmids with modified segment 7 (NSP3) cDNAs used to generate recombinant (r)SA11 viruses expressing portions of the human NoV VP1 protein. Illustration indicates nucleotide positions of the coding sequences for NSP3 (non-structural protein 3), porcine teschovirus 2A element (2A), 3×FLAG (3FL), 1×FLAG (1FL), or 6× histidine (6×His) tag, and/or thrombin cleavage site (Th) and complete VP1, or subdomains of P2 and P. The red arrow notes the position of the 2A translational stop-restart site, and the asterisk notes the end of the ORF (open reading frame). Sizes (expressed as the number of amino acids encoded (aa) of encoded NSP3 and VP1 products are given in parenthesis. T7 (T7 RNA polymerase promoter sequence), Rz (hepatitis D virus ribozyme), UTR (untranslated region).

FIG. 3A. Properties of rSA11 viruses expressing FLAG-tagged regions of the NoV VP1 protein. Double-stranded RNA was recovered from rSA11-infected MA104 cells, resolved by gel electrophoresis, and detected by ethidium-bromide staining. The genome segments of rSA11/wt (wt, wildtype) are labeled 1 to 11. Sizes (kilobasepair, kbp) of modified segment 7 RNAs (black arrows) are indicated.

FIG. 3B. Plaque assays were performed using MA104 cells and detected by crystal-violet staining.

FIG. 3C. Mean diameter values of rSA11 plaques are shown along with 95% confidence intervals (black lines).

FIG. 3D. Titers reached by rSA11 isolates were determined by plaque assay (plaque-forming units, PFU).

FIG. 3E. Whole cell lysates (WCL) were prepared from MA104 cells infected with rSA11 viruses and examined by immunoblot assay using FLAG antibody to detect VP1 protein products (P2, P, VP1 and 2A read-through products [red asterisk] and antibodies specific for RV NSP3 and VP6, porcine teschovirus 2A element, and b-actin. Sizes (kDa) of protein molecular weight markers (MWM) are indicated.

FIG. 4A. Properties of rSA11 viruses expressing His-tagged NoV capsid proteins. dsRNAs were recovered from MA104 cells infected with plaque-purified rSA11 isolates expressing His-tagged proteins, resolved by gel electrophoresis, and detected by ethidium-bromide staining. RNA segments of rSA11/wt are labeled 1 to 11. Sizes (kbp) of modified segment 7 RNAs (black arrows) of rSA11 isolates are indicated.

FIG. 4B. Plaque assays were performed using MA104 cells and detected by crystal-violet staining.

FIG. 4C. Mean diameter values of plaques, noting 95% confidence intervals (black lines).

FIG. 4D. Titers reached by rSA11 isolates were determined by plaque assay.

FIG. 4E. Whole cell lysates (WCL) were prepared from MA104 cells infected with rSA11 viruses and examined by immunoblot assay using anti-6×His antibody to detect VP1 protein products (P and VP1) and antibodies specific for RV NSP3, VP6, porcine teschovirus 2A element (2A), and cellular beta actin. Sizes (kilodaltons) of protein markers (MWM) are indicated.

FIG. 5A. Properties of rSA11/RIX NSP3 virus expressing NoV P protein dsRNA was recovered from MA104 cells infected with plaque-purified rSA11 and rSA11/RIX NSP3-2A-P His isolates (plaques 1-3), resolved by gel electrophoresis, and detected by ethidium-bromide staining. RNA segments of rSA11/wt. are labeled 1 to 11. Sizes (kbp) of modified segment 7 RNAs (black arrows) of rSA11 isolates are indicated.

FIG. 5B. Plaque assays were performed using MA104 cells and detected by crystal-violet staining.

FIG. 5C. Titers reached by rSA11 isolates were determined by plaque assay.

FIG. 5D. Whole cell lysates (WCL) were prepared from MA104 cells infected with rSA11 and rSA11/RIX NSP3-2A-P His isolates and examined by immunoblot assay using anti-6×His antibody to detect the P protein and 2A read-through products, and antibodies specific for RV SA11 NSP3, VP6, porcine teschovirus 2A element (2A), and cellular beta actin.

FIG. 6A. Dimerization of NoV capsid proteins expressed by rSA11. (A) MA104 cells were mock infected or infected with rSA11/wt or rSA11/NSP3-2A-fP2, rSA11/NSP3-2A-fP, rSA11/NSP3-2A-fVP1 and rSA11/NSP3-2A-VP1f, incubated until 9 h. p, i, when cells were harvested. Cell lysates were mixed with sample buffer containing sodium dodecyl sulfate and β-mercaptoethanol, incubated for 10 min at either 25° C. or 95° C., resolved by electrophoresis on a Biorad 4 to 20% polyacrylamide gel, and blotted onto a nitrocellulose membrane. Blots were probed with M2 FLAG antibody, guinea pig polyclonal anti-NSP3 or anti-VP6 antibodies or with a rabbit anti-B actin monoclonal antibody. Primary antibodies were detected using HRP-conjugated secondary antibodies. Sizes (kilodaltons, kDa or kD) of molecular weight protein markers (MWM) are indicated.

FIG. 6B. MA104 cells were mock infected or infected with rSA11/wt or rSA11/NSP3-2A-PHis, rSA11/NSP3-2A-VP1His, rSA11/NSP3-2A-VP1ThHis for 9 hpi, and cells were harvested. Cell lysates were prepared as above, resolved by electrophoresis on a Biorad 4 to 20% polyacrylamide gel, and blotted onto a nitrocellulose membrane. Blots were probed with mouse anti-6×His antibody, guinea pig polyclonal anti-NSP3 or anti-VP6 antibodies or with a rabbit anti-B actin monoclonal antibody. Primary antibodies were detected using HRP-conjugated secondary antibodies. Sizes (kilodaltons) of protein markers (MWM) are indicated.

FIG. 7A. NoV VP1 capsid proteins expressed from rSA11 strains folded properly. (A) Lysates prepared from MA104 cells infected with rSA11/wt, rSA11/NSP3-2A-fVP1, rSA11/NSP3-2A-VP1f, and rSA11/NSP3-2A-VP1His viruses were analyzed by immunoprecipitation (IP) assays using a NoV VP1 specific monoclonal antibody (anti-NoV GII.4 NVB43.9, Absolute antibody) recognizing a conformational dependent epitope of the folded VP1 proteins. Antigen-antibody complexes were recovered using magnetic IgA/G beads, resolved by gel electrophoresis, and blotted onto nitrocellulose membranes. Blots were probed with anti-FLAG and anti-6×His antibodies to detect immunoprecipitated VP1 proteins, and guinea pig polyclonal anti-NSP3 or anti-VP6 antibodies or with a rabbit anti-B actin monoclonal antibody. Blue arrows indicate immunoprecipitated VP1 proteins. Ig light chain, (Ig/L) and Ig heavy chain, Ig/H.

FIG. 7B. Lysates were similarly analyzed with a NSP2-specific mouse monoclonal antibody (#171), and blots were probed with mouse anti-NSP2 antibody (#516) to detect immunoprecipitated NSP2 proteins, and guinea pig polyclonal anti-NSP3 or anti-VP6 antibodies or with a rabbit anti-B actin monoclonal antibody. Positions of molecular weight markers (MWM) in kDa are indicated.

FIG. 8A. Genetic stability of rSA11 strains expressing NoV proteins. rSA11 strains were serially passaged 5 times (P1 to P5) in MA104 cells. (A) Genomic RNAs were recovered from infected cell lysates and analyzed by gel electrophoresis. Positions of viral genome segments are labeled. Position of modified segment 7 (NSP3) dsRNAs introduced into rSA11 strains are denoted with black arrows.

FIGS. 8B and 8C. Genomic RNAs were recovered from cells infected with lysates at three dilutions (1:10, 1:100 and 1:1000) and analyzed by gel electrophoresis. Genetic instability of the modified segment 7 (NSP3) dsRNA of rSA11/NSP3-2A-fVP1 and rSA11/NSP3-2A-VP1His yielded re-arranged RNAs during serial passage (shown as red arrows).

FIGS. 8D. and 8E. Genomic RNAs prepared from large (L) and small(S) plaques isolated from P5 lysates of rSA11/NSP3-2A-fVP1 and rSA11/NSP3-2A-VP1His viruses. The re-arranged segment 7 RNAs are denoted as fVP1/R1, fVP1/R2 and VP1 His/R (red arrows).

FIG. 8F. Organization of re-arranged segment 7 RNA (R) sequences determined by sequencing the cDNAs prepared from re-arranged RNAs. Sequence deletions are indicated with dashed lines.

FIG. 9A. Characterization of rSA11 variants generated from rSA11 viruses modified to express NoV VP1. Genomic RNAs were recovered from rSA11/NSP3-2A-fVP1 and rSA11/NSP3-2A-VP1His infected cell lysates and analyzed by gel electrophoresis. Positions of viral genome segments are labeled. Position of modified segment 7 (NSP3) dsRNAs introduced into rSA11 strains are denoted with black arrows and generation of random variants are shown with red arrows.

FIG. 9B. Whole cell lysates (WCL) were prepared from MA104 cells infected with variant rSA11 viruses and examined by immunoblot assay using antibodies specific for RV NSP3, VP6, and cellular beta actin. Probing with NSP3 antisera shows multiple NSP3 protein bands.

FIGS. 9C., 9D., 9E., 9F., 9G., and 9H. Genomic RNAs were recovered from cells infected with large (L) and small(S) plaque isolates from fVP1variant pools (V1-V4) and VP1His variant pools (V5-V6) and analyzed by gel electrophoresis (V: Variant).

FIG. 9I. The organization of re-arranged segment 7 RNA (denoted as R) sequences determined by sequencing of cDNAs made from variant segment 7 RNAs. Sequence deletions are indicated with dashed lines. The sizes of re-arranged variant segments are shown in parenthesis.

FIG. 10A., 10B. Impact of genome size on RV particle density. (A-B). MA104 cells were infected with rSA11/wt, rSA11/NSP3-fP2, rSA11/NSP3-fP, rSA11/NSP3-fVP1, rSA11/NSP3-PHis, rSA11/NSP3-VP1His viruses at an MOI of 5. At 12 hours p.i. (post-infection), the cells were recovered, lysed by treatment with non-ionic detergent, and treated with EDTA to convert RV virions into DLPs. DLPs were banded by centrifugation in CsCl gradients and their densities (g/cm³) determined using a refractometer. The particle densities are shown in the Tables embedded in FIG. 10A., 10B.

FIG. 10C. and FIG. 10D. Electrophoretic profile of the dsRNA genomes of DLPs recovered from CsCl gradients. Panel C RNAs derive from DLPs in panel A (FIG. 10A) and panel D (FIG. 10C) RNAs derive from DLPs in panel B. RNA segments of rSA11/wt. are labeled, 1 to 11. Positions of modified segment 7 RNAs are indicated with black arrows and red arrow indicated variants generated in the sample.

FIG. 11. Plasmids with modified segment 7 (NSP3) cDNAs used to generate rSA11s encoding the SARS-CoV-2 S1 protein. Schematic indicates nucleotide positions of the coding sequences for NSP3, porcine teschovirus 2A element, 3× or 1×FLAG (FL), 6×His (His) and the complete S1. The red arrow notes the position of the 2A translational stop-restart site, and the asterisk notes the end of the ORF. Sizes (in terms of the number of amino acids encoded the sequence (aa)) of encoded NSP3 and S1 proteins are shown in parenthesis. T7 (T7 RNA polymerase promoter sequence), Rz (Hepatitis D virus ribozyme), UTR (untranslated region).

FIG. 12A. Properties of rSA11 viruses expressing SARS CoV-2 S1 protein. dsRNA was recovered from MA104 cells infected with plaque-purified rSA11 isolates, resolved by gel electrophoresis, and detected by ethidium-bromide staining. RNA segments of rSA11/weight (wt.) are labelled 1 to 11. Sizes (Kbp) of segment 7 RNAs (black arrows) of rSA11 isolates are indicated.

FIG. 12B. Cell lysates were prepared from cells infected with rSA11 viruses 9, hours post infection (h p.i.), and examined by immunoblot assay using anti-FLAG and anti-6×His antibodies to detect S protein and the same blot re-probed with antibodies specific for SARS CoV-2 S1 protein, RV NSP3 and VP6, and cellular beta actin.

FIG. 12C. Plaque assays were performed using MA104 cells and detected by crystal-violet staining.

FIG. 12D. Titers reached by rSA11 isolates were determined by plaque assay.

FIG. 13. SARS CoV-2 S1 proteins expressed from rSA11 viruses are glycosylated. Whole cell lysates were prepared from cells infected with rSA11 viruses 9 h.p.i are treated with or without Endo H reagent and examined by immunoblot assay using anti-FLAG and anti-6×His antibodies to detect S1 protein, and the same blot is re-probed for an antibody specific for SARS CoV-2 S1. Immunoblot was also probed with antibodies specific for RV NSP3 and VP6, and the same blot is re-probed with antibodies specific for cellular beta actin.

FIG. 14A. and FIG. 14B. SARS CoV-2 S1 proteins expressed from rSA11 viruses are able to bind to human ACE-2 receptor. Lysates prepared from MA104 cells infected with rSA11/wt. and rSA11/NSP3-2A-S1 viruses were examined by co-immunoprecipitation assay using a recombinant ACE-2-human IgG protein. The protein-antibody complexes were recovered using protein IgA/G beads, resolved by gel electrophoresis, blotted onto nitrocellulose membranes, and probed with anti-FLAG and anti-6×His antibodies, SARS CoV-2 S1 antibody, antibodies specific for RV NSP3 and VP6, and cellular beta actin.

FIG. 15. Localization of SARS CoV-2 S1protein in RV-infected cells. MA104 cells were mock infected or infected with recombinant SA11 viruses: wt, NSP3-2A-S1f. At 9 h p.i., cells were fixed with 3.7% formaldehyde. Afterwards, all cells were incubated with Rabbit S1 antibody, mouse NSP2 antibody, followed by Alexa 488 anti-rabbit IgG (green) and TRITC anti-mouse IgG (red) and to detect locations of S1 protein and NSP2 proteins in the infected cells. Nuclei were detected by staining with DAPI. Cells were analyzed with an Echo Revolve fluorescence microscope (20× objective) using fluorescein isothiocyanate and tetramethyl rhodium isothiocyanate windows.

FIG. 16A. Genetic stability of rSA11 strains expressing SARS CoV-2 S1 f protein. rSA11 strain expressing S1 protein was serially passaged 5 times (P1 to P5) in MA104 cells. Genomic RNAs were recovered from infected cell lysates and analysed by gel electrophoresis. Positions of viral genome segments are labelled. Positions of modified segment 7 (NSP3) dsRNAs introduced into rSA11 strain are denoted with black arrows.

FIG. 16B. Lysates prepared from MA104 cells infected with rSA11/wt and serially passed SA11/NSP3-2A-S1f viruses (P1 to P5) were examined by immunoblot assay and probed with FLAG antibody, SARS CoV-2 S1 antibody, antibodies specific for RV NSP3 and VP6, and cellular beta actin.

### DETAILED DESCRIPTION

Genetic analysis of the entire genome or individual virus genes has led to the identification of seven NoV genogroups, and sequence studies on capsid protein VP1 and RNA-dependent RNA polymerase have identified more than 30 genotypes (7). The NoV disease that occurs in humans is mostly due to the strains included in Genogroups I (GI), II (GII), and IV (GIV) (11). However, GII is the predominant genogroup causing human disease worldwide. The GII.4 genotype has dominated since the 1990s, accounting for 55-85% of all human NoV disease (12-14). There is high genetic diversity within the genogroup and infection due to strains included in one genogroup generally do not confer protection against another genogroup, which makes it difficult to develop an effective universal vaccine (15).

The NoV genome is approximately 7.6 kbp, contains three open reading frames (ORF 1-3), and ORF-3 encodes the major structural protein, VP1. The viral capsid is comprised of 90 dimers of VP1, which is comprised of a shell(S) domain and a protruding (P) domain (16). The S domain is the most highly conserved VP1 domain, whereas the P domain is more variable and includes a P1 and a P2 subdomain that are discontinuous in their primary amino acid sequence. The highly variable P2 subdomain represents the immunodominant region of the protein, a target for neutralizing antibodies, and contains the defined receptor binding site for NoV (17, 18). The immunological response following NoV infection can be identified by measuring the serum human histo-blood group antigens (19, 20).

Three types of NoV vaccines have been developed; non-replicating virus-like particles (VLPs), P particles, and recombinant adenovirus vectored vaccines (21-23). Most vaccine studies were performed in adults (24-26), while a phase II trial of a GI.1 and GII.4 bivalent vaccine trial carried out in children and infants for testing the immunogenicity and safety showed that the preparation has evoked a robust immune response (27). In an attempt to simultaneously prevent both RV and NoV diseases, a trivalent vaccine containing two NoV VLPs (GII.4 and GI.3) and the oligomeric RV VP6 was tested in animal models (28, 29). RV VP6 is a highly conserved protein, capable of evoking a significant immune response that protects from RV infection, and it can act as an adjuvant increasing the immune response against NoV antigen (30).

Recent development of RV reverse genetics systems has resulted in the generation of recombinant RVs as expression vectors of foreign proteins (31-36). The RV genome consists of 11 segments of dsRNA, with a total size of 18.5 kbp. All the segments contain a single ORF except for segment 11. These encode the six structural (VP) or six non-structural (NSP) viral proteins (37). Genome segment 7 of group A RV (RVA) encodes 36 kDa protein, NSP3, an RNA binding protein that acts as a translation enhancer of viral (+) mRNAs in infected cells (38, 39).

To use RV as a vaccine expression vector, the inventors explored the possibility of making an RV-SARS-CoV-2 vaccine, by modifying the NSP3 ORF using a 2A translation element to express regions of SARS-CoV-2 spike proteins (31). Well growing, genetically stable recombinant RVs that express domains of SARS CoV-2 S protein were made, and the NSP3 product of these viruses was functional, capable of dimerization and inducing nuclear localization of the cellular poly(A)-binding protein (31, 32, 40). Therefore, the inventor's developed RV as an effective vector system for making a combined RV-NoV vaccine that can induce immunological protection against both RV and NoV infection.

### Polynucleotides

In an aspect of the current disclosure, polynucleotides comprising: a sequence encoding a rotavirus (RV) NSP3 protein; and a heterologous polynucleotide are provided. The inventors disclose herein that the polynucleotides, in some embodiments, encode a positive sense viral transcript and may be expressed in a cell to generate functional gene products. Therefore, in some embodiments, the polynucleotides are operably linked to a promoter, e.g., a T3 promoter (SEQ ID NO: 31) or a T7 promoter (SEQ ID NO: 30).

As used herein, “operably linked” refers to a functional relationship between two or more nucleic acid (e.g., DNA) segments. Typically, it refers to the functional relationship of transcriptional regulatory element (promoter) to a transcribed sequence. For example, a promoter is operably linked to a coding sequence if it stimulates or modulates the transcription of the coding sequence in an appropriate cell. Generally, promoter transcriptional regulatory elements that are operably linked to a sequence are physically contiguous to the transcribed sequence, i.e., they are cis acting. However, some transcriptional regulatory elements, such as enhancers, need not be physically contiguous or located in close proximity to the coding sequences whose transcription they enhance. Exemplary promoters include a T7 bacteriophage promoter (SEQ ID NO: 14) and a T3 bacteriophage promoter (SEQ ID NO: 15). A suitable promoter may be chosen by from promoters known in the art. In some embodiments, the cells are mammalian cells and are selected from MA-104 cells, Vero cells and BHK-1 cells.

In some embodiments, the RV NSP3 protein is SEQ ID NO: 1, or a sequence with at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to SEQ ID NO: 1.

In some embodiments, the sequence encoding NSP3, e.g., SEQ ID NO: 9, further comprises the heterologous polynucleotide fused to the 3′ end of the sequence such that the heterologous polynucleotide encodes a protein or peptide in frame with the NSP3 sequence, thereby allowing transcription of a single mRNA that encodes both NSP3 and the heterologous polynucleotide. In some embodiments, the polynucleotide comprising a sequence encoding NSP3 and a heterologous polynucleotide comprise a sequence encoding a cleavage site. In some embodiments, the cleavage site is a self-cleaving peptide, e.g., porcine teschovirus P2A element (SEQ ID NO: 13). Thus, in some embodiments, the disclosed compositions comprise, from 5′ to 3′, a polynucleotide encoding NSP3, fused in-frame to a sequence encoding a self-cleaving peptide which is fused in-frame to a heterologous polynucleotide sequence encoding a peptide or protein. Accordingly, transcription and translation of such compositions results in production of a fusion protein comprising, from N- to C-terminus, an RV NSP3 protein fused to a self-cleaving peptide, e.g., SEQ ID NO: 13, which is fused to a peptide or protein encoded by the heterologous polynucleotide, in a cell; following translation, the fusion protein self-cleaves resulting in two separate proteins (1) a functional RV NSP3 protein and (2) the protein or peptide encoded by the heterologous polynucleotide. In some embodiments, the compositions further comprise a sequence encoding a sequence encoding a linker, e.g., a flexible linker located 3′ to, and in frame with, the sequence encoding NSP3 protein and 5′ to a cleavage site. Without being limited by any theory or mechanism, the inventors believe that the addition of a flexible linker between the NSP3 protein and the cleavage site improves cleavage. In some embodiments, the linker is a (GAG) n linker (also referred to as a GAG linker), wherein n=1, 2, 3, 4, 5, 6, 7, 8, 9, 10, or more, or a (GSG) n linker (also referred to as a GSG linker), wherein n=1, 2, 3, 4, 5, 6, 7, 8, 9, 10 or more.

In some embodiments, the sequence encoding a cleavage site encodes a protease cleavage site, e.g., a thrombin cleavage site, e.g., SEQ ID NO: 12.

In some embodiments, the heterologous polynucleotide comprises a sequence encoding at least one carrier moiety. In some embodiments, the carrier moiety is a secretion peptide, e.g., interleukin-2-secretion protein, SARS CoV-2 S1 secretion peptide, or a ligand for cell surface receptor, e.g., immunoglobulin IgG, fetal receptor FcRn.

The inventors further contemplate that the heterologous polynucleotide sequence described above comprise sequences encoding proteins or peptides derived from infectious organisms, e.g., NoV or SARS-CoV-2. Therefore, in some embodiments, the disclosed compositions comprise sequences encoding RV NSP3 fused, in-frame, to a heterologous polynucleotide encoding a NoV protein or peptide, e.g., NoV.

In some embodiments, the NoV VP1 protein has an amino acid sequence selected from SEQ ID NOs: 24 or 26. In some embodiments the heterologous polynucleotide encodes NoV VP1 and comprises SEQ ID NO: 25. In some embodiments, the norovirus protein is selected from SEQ ID NOs: 24 or 26 and 80-84.

The inventors further disclose herein exemplary rotaviral NSP3 noroviral fusion proteins. Accordingly, in another aspect of the current disclosure, further polynucleotides are provided. In some embodiments, the polynucleotides encode a protein selected from SEQ ID NOs: 2-11, or a sequence with at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a sequence selected from SEQ ID NOs: 2-11.

In some embodiments, the polynucleotides comprise SEQ ID NOs: 13-22 or a sequence with at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to any one of SEQ ID NOs: 13-22.

### Infectious Particles

In another aspect of the current disclosure, infectious particles are provided. In some embodiments, the infectious particles comprise a sequence encoding a rotavirus (RV) NSP3 protein; and a heterologous polynucleotide. In some embodiments, the infectious particles are made by introducing a polynucleotide comprising a sequence encoding an RV NSP3 protein; and a heterologous polynucleotide into a cell.

As used herein, “infectious particles” refers to any particle capable of causing an infection of an organism or cell. Exemplary infectious particles include, but are not limited to, viral particles, virions, and the like. The terms “virus,” “viral particle,” and “virion” may be used interchangeably herein.

In some embodiments the infectious particles are an RV, e.g., RV strain SA11.

Without wishing to be limited, the instant disclosure provides compositions comprising polynucleotides encoding RV proteins operably linked to a promoter, e.g., a T7 promoter (SEQ ID NO: 30). In some embodiments, the compositions may be used in a reverse genetics approach to generate recombinant RV, e.g., recombinant RV strain RIX4414. Thus, some embodiments, the recombinant RV may comprise the disclosed compositions.

In some embodiments, the cells are selected from BHK-1 cells, MA-104 cells, and Vero cells. In some embodiments, the cells are BHK-1 cells expressing T7 polymerase, also known as BHK-T7 cells.

### Pharmaceutical Compositions

The inventors disclose herein compositions, methods, and systems useful in making recombinant rotavirus (RV) that may be suitable for administration to subjects. Therefore, in another aspect of the current disclosure, pharmaceutical compositions are provided. In some embodiments, the pharmaceutical compositions comprise an infectious particle comprising a polynucleotide comprising a sequence encoding an RV NSP3 protein; and a heterologous polynucleotide. In some embodiments, the pharmaceutical compositions comprise an infectious particle made by transfecting a cell with a polynucleotide comprising a sequence encoding a recombinant RV NSP3 protein and a heterologous polynucleotide.

The compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed composition, which effective amount is related to the daily dose of the composition to be administered. Each dosage unit may contain the daily dose of a given composition or each dosage unit may contain a fraction of the daily dose, such as one-half or one-third of the dose. The amount of each composition to be contained in each dosage unit can depend, in part, on the identity of the particular composition chosen for the therapy and other factors, such as the indication for which it is given. The pharmaceutical compositions disclosed herein may be formulated so as to provide quick, sustained, or delayed release of the active ingredient after administration to the patient by employing well known procedures.

The pharmaceutical compositions may be utilized in methods of eliciting an immune response or vaccinating against a pathogen, e.g., RV, NoV, SARS-CoV-2. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration. By way of example, a subject may be at risk for infection by a pathogen, e.g., RV, NoV, and administration of the disclosed pharmaceutical compositions elicits a protective immune response or vaccinates against the pathogen.

As used herein the term “effective amount” refers to the amount or dose of the compositions, upon single or multiple dose administration to the subject, which provides the desired effect in the subject under diagnosis or treatment. The disclosed methods may include administering an effective amount of the disclosed compositions (e.g., as present in a pharmaceutical composition) for eliciting an immune response to a pathogen, e.g., RV, NoV, SARS-CoV-2, or vaccinating against the pathogen.

An effective amount can be readily determined by the attending diagnostician, as one skilled in the art, by the use of known techniques and by observing results obtained under analogous circumstances. In determining the effective amount or dose of composition administered, a number of factors can be considered by the attending diagnostician, such as: the species of the subject; its size, age, and general health; the degree of involvement or the severity of the disease or disorder involved; the response of the individual subject; the particular composition administered; the mode of administration; the bioavailability characteristics of the preparation administered; the dose regimen selected; the use of concomitant medication; and other relevant circumstances.

Oral administration is an illustrative route of administering the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Methods of Generating Recombinant Rotavirus (RV)

The inventors have generated recombinant RVs by using a reverse genetics system in the SA11 background by introducing polynucleotides encoding NSP3 fused to additional heterologous proteins, e.g., NoV proteins. Therefore, in another aspect of the current disclosure, methods of generating RV in vitro are provided. In some embodiments, the methods comprise: introducing a polynucleotide comprising a sequence encoding an RV NSP3 protein; and a heterologous polynucleotide into a cell; allowing the cell to express the polynucleotide; incubating the cells for a sufficient time to produce RV; and harvesting virus produced by the cells to generate RV in vitro. To successfully generate a competent RV, each of the 11 RV genome segments must be expressed in a cell. Therefore, in some embodiments, the methods further comprise introducing one or more additional polynucleotides into the cell before the allowing step, wherein the one or more additional polynucleotides comprise a sequence encoding an RV protein selected from VP1, VP2, VP3, VP4, VP6, VP7, NSP1, NSP2, NSP3, NSP4, and NSP5, wherein each sequence encoding an RV protein is operably linked to a promoter. In some embodiments, the one or more additional polynucleotides comprise a sequence encoding an RV protein selected from VP1, VP2, VP3, VP4, VP6, VP7, NSP1, NSP2, NSP4, and NSP5. In some embodiments, the one or more additional polynucleotides are 10 separate polynucleotides which comprise a sequence encoding an RV protein selected from VP1, VP2, VP3, VP4, VP6, VP7, NSP1, NSP2, NSP4, and NSP5, respectively. Thus, in some embodiments, the methods comprise introducing a polynucleotide comprising sequences encoding each of the 11 rotaviral proteins, which, in some embodiments, are each encoded on separate polynucleotides. In some embodiments, the one or more additional polynucleotides comprise a sequence encoding a capping enzyme operably linked to a promoter. In some embodiments, the capping enzyme is African swine fever virus capping enzyme (encoded by SEQ ID NO: 27).

### Cells

The inventors disclose herein cells comprising the disclosed polynucleotides, which may also be used in the disclosed methods and systems. Accordingly, in another aspect of the current disclosure, cells are provided. In some embodiments, the cells comprise a polynucleotide comprising a sequence encoding a recombinant RV NSP3 protein; and a heterologous polynucleotide. In some embodiments, the cells are selected from MA-104 cells, Vero cells, and BHK-1 cells.

RV vaccine strains have been traditionally grown using Vero cells. This method of producing RV has been found to be suitable for generation of RV for administration to subjects. Therefore, in some embodiments, the cells are Vero cells.

In some embodiments, the cells disclosed herein further comprise a heterologous RNA polymerase, wherein the heterologous RNA polymerase binds to the promoter in the disclosed compositions and catalyzes sequence-dependent RNA polymerization based on the polynucleotides when the polynucleotides are introduced into the cell. As used herein, “heterologous RNA polymerase” refers to an RNA polymerase introduced into a cell through molecular biological techniques, e.g., transduction, transfection, lipofection, etc. In some embodiments, the heterologous RNA polymerase comprises T7 bacteriophage RNA polymerase or T3 bacteriophage RNA polymerase, more commonly known as simply T7 polymerase and T3 polymerase, respectively.

Accordingly, in some embodiments, the cells further comprise T7 RNA polymerase or T3 RNA polymerase. In some embodiments, such cells are referred to as, e.g., BHK-T7 cells, because they are derived from BHK-1 cells, but express the heterologous RNA polymerase T7 bacteriophage RNA polymerase. Thus, as used herein, “BHK-T7 cells” are BHK-1 cells that express the heterologous RNA polymerase T7 bacteriophage RNA polymerase.

### Methods of Eliciting an Immune Response

The instant disclosure provides pharmaceutical compositions comprising infectious particles which may comprise heterologous antigens that, when administered to a subject, may be protective against natural infection with the pathogen from which the antigens are derived. Therefore, in another aspect of the current disclosure, methods of eliciting an immune response to one or more pathogens are provided. In some embodiments, the methods comprise administering a pharmaceutical composition comprising an infectious particle comprising a polynucleotide comprising a sequence encoding an RV NSP3 protein; and a heterologous polynucleotide to a subject to elicit an immune response to one or more pathogens.

In some embodiments, the methods comprise administering a pharmaceutical composition comprising an infectious particle made by transfecting cells with a polynucleotide comprising a sequence encoding an RV NSP3 protein; and a heterologous polynucleotide to a subject to elicit an immune response to one or more pathogens.

As used herein, “eliciting an immune response” refers to generation of an inflammatory reaction, increase in activation level or number of innate or adaptive immune cells in response to administration. Eliciting an immune response may also be measured as increased humoral immunity against an administered antigen in a subject. Suitable assays to measure both increased innate/adaptive cell activation and number and/or humoral immunity against an antigen are known in the art. For example, cellular immunity may be measured by increased numbers of activated T cells in the subject by, e.g., flow cytometry. Elicitation of a humoral immune response may be measured by antibody binding to the antigen administered to the subject, for which numerous methods are known in the art.

### Methods of Vaccinating a Subject

In another aspect of the current disclosure, methods of vaccinating a subject against one or more pathogens are provided. In some embodiments, the methods comprise administering a pharmaceutical composition comprising an infectious particle comprising a polynucleotide comprising a sequence encoding a rotavirus (RV) NSP3 protein; and a heterologous polynucleotide. In some embodiments, the methods comprise administering a pharmaceutical composition comprising an infectious particle made by transfecting cells with a polynucleotide comprising a sequence encoding an RV NSP3 protein; and a heterologous polynucleotide.

As used herein, “vaccinating” or “vaccination” refers to administering to a subject an antigen derived from a pathogen to stimulate an immune response in the subject to the antigen, thereby providing some level of immunity to the pathogen, should the subject become infected with the pathogen. Thus, vaccination may reduce the signs or symptoms of infection by a pathogen in a vaccinated subject or may provide neutralizing immunity and prevent infection by the pathogen in a subject.

### Systems, Platforms, and Kits for Generating Recombinant Rotavirus (RV)

In another aspect of the current disclosure systems, platforms, and kits for generating recombinant RV are provided. In some embodiments, the systems, platforms, or kits comprise: a polynucleotide comprising a sequence encoding an RV NSP3 protein; and a heterologous polynucleotide; and cells capable of expressing the polynucleotides.

In some embodiments, the kits of the current disclosure comprise a polynucleotide comprising a sequence encoding an RV NSP3 protein; and a heterologous polynucleotide; and cells capable of expressing the polynucleotides. The inventors envision that the disclosed kits may contain, in some embodiments, additional reagents necessary to introduce the polynucleotides of the instant disclosure into the cells, e.g., reagents for transfection, lipofection, electroporation, transduction, etc. Therefore, in some embodiments, the disclosed kits comprise reagents for, e.g., generating recombinant rotaviruses in vitro according to the disclosed methods.

### Illustrative Embodiments

1. A recombinant RV, comprising: an RV; and an insertion of up to 1.3 kbp of foreign sequence of, wherein the RV bearing the 1.3 bp insertion is genetically stable.
2. The recombinant RV, according to embodiment 1, wherein the RV is RIX 4414.
3. The recombinant RV, according to embodiment 1 or 2, wherein the insertion encodes at least one antigen of a non-RV virus selected from the group consisting of: NoV, SARS-CoV-2, astrovirus, enterovirus, and hepatitis E.
4 The recombinant RV, according to any of embodiments 1 to 3, wherein the insertion further encodes at least one carrier moiety.
5. The recombinant RV, according to embodiment 4, wherein the at least one carrier moiety, is a secretion peptide (e.g: interleukin-2-secretion protein, SARS CoV-2 S1 secretion peptide) or ligands for cell surface receptor, immunoglobulin IgG, fetal receptor FcRn).
6. The recombinant RV, according to any of embodiments 1 to 5, wherein the insertion encodes SARS-CoV-2 S1 protein.
7. The recombinant RV, according to any of embodiments 1 to 6, wherein the insertion encodes NoV capsid protein.
8. A vaccine, comprising the recombinant RV of any one of embodiments 1 to 7.
9. The vaccine, according to embodiment 8, wherein the vaccine is for use in children.
10. The vaccine according to any of embodiments 8 to 9, wherein the vaccine further includes at least one compound that stabilizes the recombinant RV.
11. A recombinant RV, comprising: an RV; and a sequence encoding a glycosylated exogenous capsid protein, wherein the sequence is inserted into segment 7 RNA.
12. The recombinant RV, according to the eleventh embodiment, wherein the RV is rSA11.
13. The recombinant RV of embodiments 11-12, wherein the exogenous encoded glycosylated protein is SARS-CoV-2 S1.
14. The recombinant RV of embodiments 11-13, wherein the exogenous encoded protein sequence included a C-terminal 1×-FLAG-tag.
15. The recombinant RV of embodiments 11-14, further including at one of the following: a pharmaceutically acceptable excipient, stabilizer, and or carrier.
16. A composition comprising the recombinant RV of embodiment 15, further comprising an adjuvant.
17. The composition of embodiment 16, wherein the adjuvant is an immunostimulatory oligonucleotide such as CpG, a polyacrylic acid polymer, a dimethyl dioctadecyl ammonium bromide, a sterol, saponin, a monophosphoryl lipid A or analog thereof, a quartenary amine, an aluminium hydroxide composition such as an aluminium hydroxide gel, or a combination thereof.
18. The composition of any one of embodiments 15-17, wherein the composition includes at least one compound that stabilized the recombinant RV.
19. A method of treating a subject comprising administering any of the composition according to embodiments 15-19.
20. The method of embodiment 19, wherein the subject is administered the composition of any one of embodiments 15-18 at least two times separated by at least 4 weeks.
21. A cell comprising the recombinant RV of any one of embodiments 1-14.
22. The cell of embodiment 21, wherein the host cell expresses a glycosylated protein encoded by the recombinant RV.

### EXAMPLES

### Example 1—Recombinant Rotaviruses Expressing Norovirus Protein

### Materials and Methods

### Cell Culture

Embryonic monkey kidney (MA104) cells were grown in Dulbecco's Modified Eagle Medium (DMEM) containing 5% fetal bovine serum (FBS) and 1% penicillin-streptomycin (41). Baby hamster kidney cells constitutively expressing T7 RNA polymerase (BHK-T7) were provided by Dr. Ulla Buchholz, Laboratory of Infectious Diseases, NIAID, NIH, and were propagated in Glasgow minimum essential media (GMEM) containing 5% heat-inactivated FBS, 10% tryptone-peptide broth, 1% penicillin-streptomycin, 2% non-essential amino acids, and 1% glutamine (42). BHK-T7 cells were grown in a medium supplemented with 2% Geneticin (Invitrogen) with every other passage.

### Plasmid Construction

Recombinant rSA11s were prepared using the plasmids pT7/VP1SA11, pT7/VP2SA11, pT7/VP3SA11, pT7/VP4SA11, pT7/VP6SA11, pT7/VP7SA11, pT7/NSP1SA11, pT7/NSP2SA11, pT7/NSP3SA11, pT7/NSP4SA11, and pT7/NSP5SA11 [https://www.addgene.org/Takeshi_Kobayashi/] (36) and pCMV/NP868R (33). The plasmid pT7/NSP3-P2A-fUnaG was produced by fusing a DNA fragment containing the ORF for P2A-3×FL-UnaG to 3′-end of the NSP3 ORF of pT7/NSP3SA11 using a Takara In-Fusion cloning kit (32). A plasmid (pUC57/MDA145_VP1) containing a full-length cDNA of the VP1 genome segment of the NoV GII.4 MD145-12 strain (GenBank: AY032605.1) was purchased from Genewiz. The plasmids pT7/NSP3-P2A-fP2, pT7/NSP3-P2A-fP, pT7/NSP3-P2A-fVP1 were made by replacing the UnaG ORF in pT7/NSP3-P2A-fUnaG with ORFs for the P2, P, and VP1 regions, respectively, of the NoV VP1 capsid protein, by In-Fusion cloning. The backbone of the plasmids was generated through PCR amplification of pT7/NSP3-P2A-fUnaG with the primer pairs: Vector_For and Vector_Rev (Table 1). DNA fragments containing P2, P, and VP1 coding sequences were amplified from pUC57/MDA145_VP1 using the primer pairs fP2_For and fP2_Rev, fP_For and fP_Rev, fVP1_For and fVP1_Rev respectively (Table 1). The plasmids pT7/NSP3-P2A-VP1f, pT7/NSP3-P2A-P-His, pT7/NSP3-P2A-VP1-His, pT7/NSP3-P2A-VP1-Th-His were made similarly. The backbone of the plasmids was generated by amplifying pT7/NSP3-P2A-fUnaG with the primer pairs Vector P2A For and Vector P2A Rev (Table 1). DNA fragments containing VP1 with a C terminal FLAG or P or VP1 with a C terminal His tag were produced through PCR amplification of pUC57/MDA145_VP1 with the primer pairs VP1-fFor and VP1-fRev, P-His_For and P-His_Rev, VP1-ThHis For and VP1-ThHis_Rev, VP1-His_For and VP1-His_Rev respectively (Table 1). A puc19 plasmid containing a RIX/NSP3-P2A-P-His insert under the control of a T7 transcription promoter (puc19/T7/RIX/NSP3-P2A-P-His) was purchased from Bio Basic Canada Inc. Transfection quality plasmids were prepared commercially (www.plasmid.com) or using Qiagen plasmid purification kits. Primers were provided by and sequences determined by EuroFins Scientific.

### Recombinant Viruses

The reverse genetics protocol used to generate recombinant RVs was described in detail previously. Briefly, BHK-T7 cell monolayers in 12-well plates were transfected with SA11 pT7 plasmids and pCMV-NP868R using Mirus TransIT-LT1 transfection reagent. Transfection mixtures contain 0.8 ug of each of the 11 pT7 plasmids except for pT7/NSP2SA11 and pT7/NSP5SA11, which were used at levels 3-fold higher. Two days after transfection, the BHK-T7 cells were over-seeded with MA104 cells and the trypsin in the medium was adjusted to a final concentration of 0.5 ug/ml. Three days later, the BHK-T7/MA104 cell mixture was freeze-thawed 3-times and the lysates were clarified by low-speed centrifugation (800×g, 5 min). Recombinant viruses in clarified lysates were amplified by a single round of passage on MA104 monolayers and recovered by plaque purification. Viral dsRNAs were recovered from infected-cell lysates by TRIzol extraction, resolved by electrophoresis on 10% polyacrylamide gels in Tris-glycine buffer, detected by staining with ethidium bromide, and visualized using a BioRad ChemiDoc MP Imaging System.

### Plaque Assay

RV plaque assays were performed as described before. To visualize plaques, cell monolayers with agarose overlays were incubated overnight with phosphate-buffered saline (PBS) containing 3.7% formaldehyde. Afterward, agarose overlays were removed, and the monolayers were stained for 3 h with a solution of 1% crystal violet dissolved in 5% ethanol. Monolayers then were rinsed with water and air-dried. Plaque images were captured using a Bio-Rad ChemiDoc imaging system and diameters were measured using ImageJ software and the results were analyzed with GraphPad Prism, version 8. Statistical significance of plaque size differences was determined using an unpaired Student's t-test and included 95% confidence intervals.

### Immunoblot Analysis

MA104 cells were mock-infected or infected with 5 plaque-forming units (PFU) per cell of recombinant RV and harvested at 9 h p.i. Cells were washed with cold PBS, pelleted by centrifugation (5000×g, 10 min), and lysed by incubation for 30 min on ice in non-denaturing lysis buffer (300 mM NaCl, 100 mM Tris-HCl, pH 7.4, 2% Triton X-100, and 1×EDTA-free protease inhibitor cocktail [Roche Complete]). For immunoblot assays, lysates were resolved by electrophoresis on 10% polyacrylamide gels and transferred to nitrocellulose membranes. After blocking with PBS containing 5% non-fat dry milk, blots were probed with mouse monoclonal FLAG M2 (F1804, Sigma, 1:2000), mouse monoclonal anti-6×His antibody (MCA1396GA, Bio-Rad, 1:1000), mouse 2A antibody (NBP2-59627, Novus, 1:1000), guinea pig polyclonal NSP3 (Lot 55068, 1:2000), VP6 (Lot 53963, 1:2000) antisera or rabbit monoclonal B-actin (8457S, Cell Signaling Technology (CST), 1:1000) antibody. Primary antibodies were detected using 1:10,000 dilutions of horseradish peroxidase (HRP)-conjugated secondary antibodies (goat anti-mouse IgG (CST), goat anti-guinea pig IgG (KPL), or goat anti-rabbit IgG (CST) or Alexa fluor conjugated antibody (goat anti-mouse Alexa 647 antibody (CST) in 2.5% non-fat dry milk. HRP signals were developed using Clarity Western ECL Substrate (Bio-Rad) and detected using a Bio-Rad ChemiDoc imaging system, whereas Alexa fluor signals were visualized directly using a Bio-Rad ChemiDoc imaging system.

To evaluate the dimerization capacity of NoV proteins expressed by rSA11 viruses, cell lysates were adjusted to a final concentration of 1.5% sodium dodecyl sulfate and 3% β-mercaptoethanol and incubated for 10 Min at 25° C. or 95° C. Afterward, proteins in the samples were resolved by electrophoresis on 10% polyacrylamide gels and detected by immunoblot assay.

### Immunoprecipitation Assay

Whole-cell lysates (WCLs) were prepared from MA1 monolayers either mock-infected or infected with rSA11 virus at 9 hours p.i., as described above. Rabbit anti-NoV GII.4 monoclonal antibody [NVB43.9] (Ab00269-23.0, Absolute antibody, final dilution of 1:150) or NSP2 mouse polyclonal antibody (Lot 171, final dilution of 1:200) were added to cell lysates. After incubation at 4° C. with gentle rocking for 18 h, antigen-antibody complexes were recovered using Pierce magnetic IgA/IgG beads (ThermoScientific), resolved by gel electrophoresis, and blotted onto nitrocellulose membranes. Blots were probed with anti-FLAG antibody (1:2000) or anti-6×His antibody (1:1000) to detect FLAG tagged or His-tagged VP1 proteins, and NSP2 antibody (Lot #516, 1:2000) to detect NSP2 proteins.

Genetic Stability of rSA11 Viruses

Viruses were serially passaged five times on MA104-cell monolayers using 1:1000, 1:100, or 1:10 dilutions of infected cell lysates prepared in serum-free DMEM medium. The cells were freeze-thawed thrice in their medium when cytopathic effects reached the completion (4-5 days) and lysates were clarified by low-speed centrifugation. The double stranded RNAs (dsRNAs) were recovered from clarified lysates by TRIzol extraction. The purified dsRNAs were resolved by electrophoresis on 10% polyacrylamide gels, and the bands of dsRNA were detected by ethidium bromide staining.

### Isolation and Sequencing of Unstable Variants

Individual rSA11 variants were recovered from pools of the serially passaged virus by plaque isolation (41). The variants were amplified by a single round of passage on MA104 cells and their genomic dsRNA recovered by Trizol extraction. The full-length genome segment 7 RNAs in the samples were amplified with segment-specific primer pairs NSP3_5′UTR 5′GGCATTTAATGCTTTTCAGTG 3′ (SEQ ID NO: 1), and NSP3_3′UTR 5′ GGCCACATAACGCCCCTATAG 3′ (SEQ ID NO: 2), and a shorter fragment from the C terminus of NSP3 ORF to 3′UTR region was amplified with the primer pairs NSP3 C termF 5′ CATTGCACGCTTTTGATGACTTAG 3′ (SEQ ID NO: 3), and NSP3_3′UTR 5′GGCCACATAACGCCCCTATAG 3′ (SEQ ID NO: 4), similarly using Superscript III One-Step RT-PCR System with Platinum Taq DNA polymerase (Invitrogen). Amplified PCR products were resolved by electrophoresis on 0.8% agarose gels in Tris-acetate-EDTA buffer, products were gel-purified using Nucleospin gel and PCR Clean-up (Takara), and the sequences were determined by EuroFins Scientific.

### CsCl (Cesium Chloride) Gradient Centrifugation

The density of double-layered particles (DLPs) was determined as described previously. Briefly, MA104-cells in 10-cm cell culture plates were infected with 5PFU per cell of rSA11 viruses and harvested at 12 h p.i. Cells were scraped into 1 ml of PBS (phosphate buffered saline), lysed by adjusting the solution to a final concentration of 0.5% Triton X-100, and incubated on ice for 5 min. After low-speed centrifugation to remove cellular debris, the lysates were adjusted to 10 mM EDTA (ethylenediaminetetraacetic acid) and incubated for 1 h at 37° C. with intermittent mixing to convert RV virions to DLPs. CsCl was added to samples to a density of 1.367 g/cm³, and samples were centrifuged at 110,000×g in a Beckman SW55Ti rotor at 8° C. for 22 h. Virus bands were detected in the gradients using an inverted light source. Fractions containing viral bands were recovered with a micropipette and their CsCl densities were determined using a refractometer.

### GenBank Accession Numbers

Segment 7 sequences in rSA11 viruses have been deposited in GenBank: wt. (LC178572), NSP3-P2A-NoVfP2 (MN190002), NSP3-P2A-NoVfP (MN190003), NSP3-P2A-NoVfVP1 (MN190004), NSP3-P2A-NOV VP1f (MN201548), NSP3-P2A-NOV P-His (MN201549), NSP3-P2A-NOV VP1-ThHis (MN201547), NSP3-P2A-NOV VP1-His (MZ562305), RIX/NSP3-P2A-NOV P-His (MZ643978). See also Table 2.

### Results

### Modified Genome Segment 7 Expressing NoV Capsid Proteins

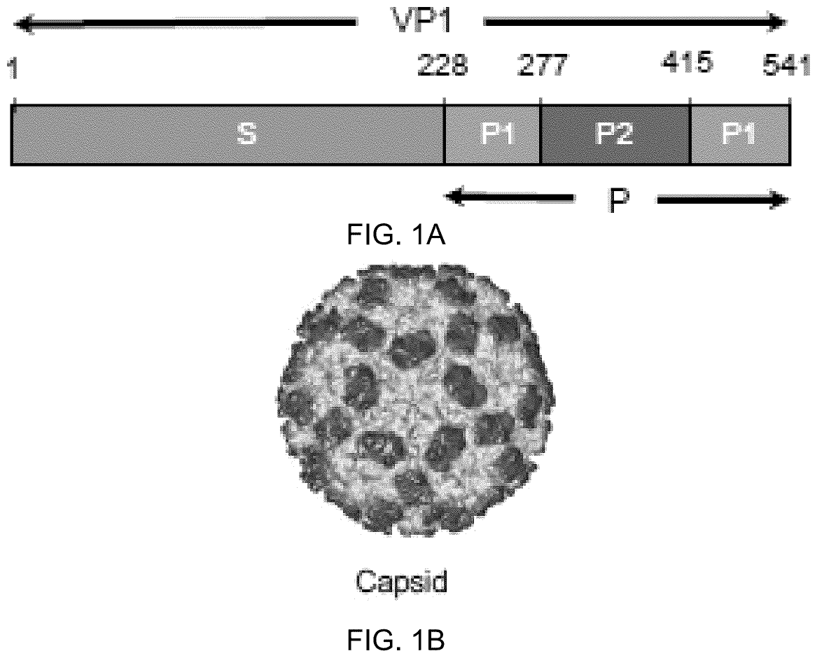

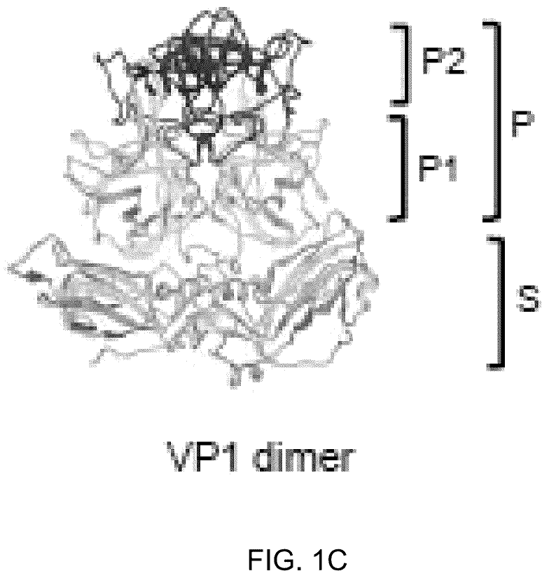

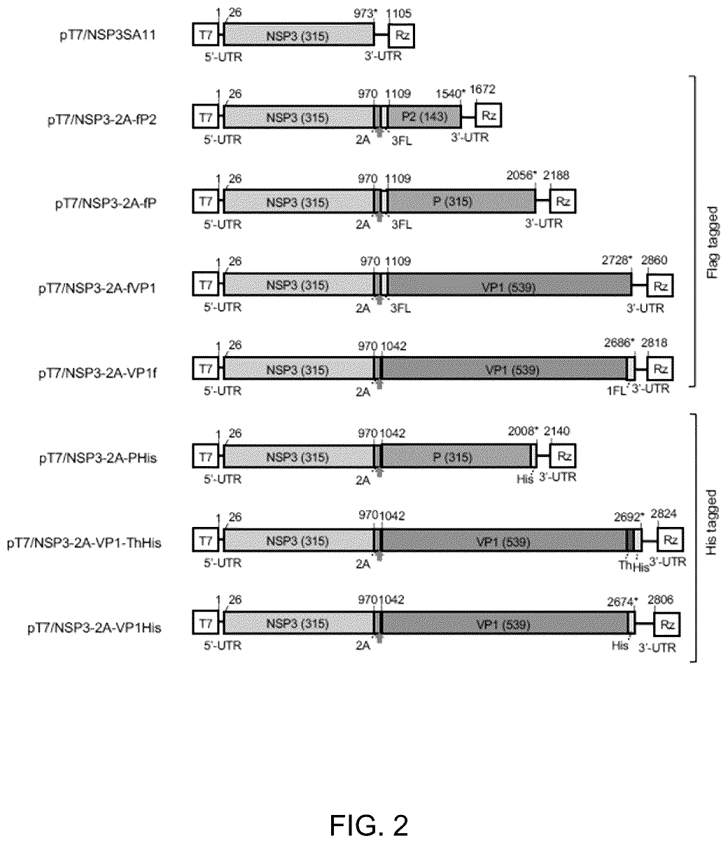

For generating RV as expression vectors for regions of NoV capsid proteins (FIG. 1), genome segment 7 in pT7/SA11 vectors was modified by replacing the wt. NSP3 ORF with a cassette comprising of NSP3 ORF fused to a GAG flexible linker along with porcine teschovirus 2A element (P2A) and the coding region of NoV capsid proteins (FIG. 2). The modified ORF also contained either a 3×FLAG tag (f) at the N-terminus or a 1×FLAG tag (f) at the C-terminus of a NoV protein (FIG. 2). The NoV sequences were inserted into the pT7/NSP3 plasmid at the same site as used for the generation of rSA11 strains expressing fluorescent proteins and SARS CoV-2 spike proteins. Similarly, pT7 SA11 NSP3 vectors expressing 6×His-tagged NoV proteins were generated by replacing the FLAG tagged NoV sequences with 6×His tagged NoV sequences, with or without inserting Th cleavage site (FIG. 2). This approach resulted in the development of set of pT7/SA11NSP3-2A-NOV vectors that contained FLAG or 6×His-tagged coding sequences for NoV proteins, P2 (pT7 NSP3-2A-fP2), P (pT7 NSP3-2A-fP or pT7 NSP3-2A-PHis), VP1 (pT7 NSP3-2A-fVP1, pT7 NSP3-2A-VP1f, pT7 NSP3-2A-VP1-ThHis and pT7 NSP3-2A-VP1 His) (FIG. 2). Next, to test the possibility of generating human strain genome segment 7 as an expression platform, the current neonatal RV vaccine strain Rotarix genome segment 7 (RIX NSP3) was modified to express NoV PHis protein down-stream of 2A peptide, pUC 19/RIX NSP3-2A-P His was synthesized.

Recovery and Characterization of rSA11 Viruses Expressing FLAG Tagged NoV Capsid Proteins

Recombinant SA11 viruses expressing NoV capsid proteins were generated by transfecting BHK-T7 cells with a complete set of eleven pT7 plasmids encoding +mRNA of RV genome segments and a CMV expression vector encoding African swine fever virus capping enzyme (NP8688R), and the pT7/NSP3SA11 was replaced with a pT7/NSP3-2A-NOV vector as described before. The transfected BHK-T7 cells were over-seeded with MA104 cells 2 days post-transfection and the cell mixture was freeze-thawed three days later, and the recombinant RVs were recovered by growing them on MA104 cells. The rSA11 isolates were plaque purified and amplified to larger volumes before characterization. The characteristics of rSA11 viruses are summarized in Table 2.

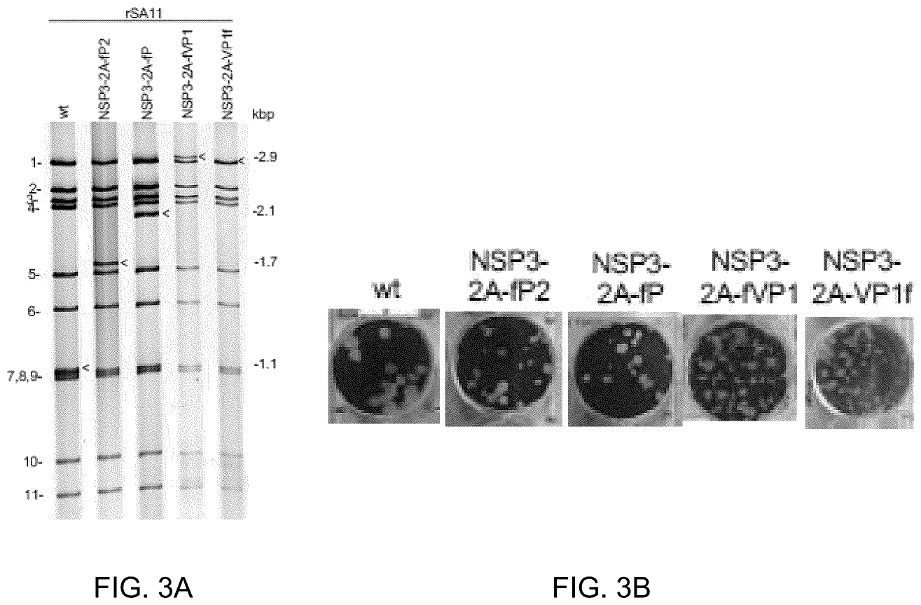

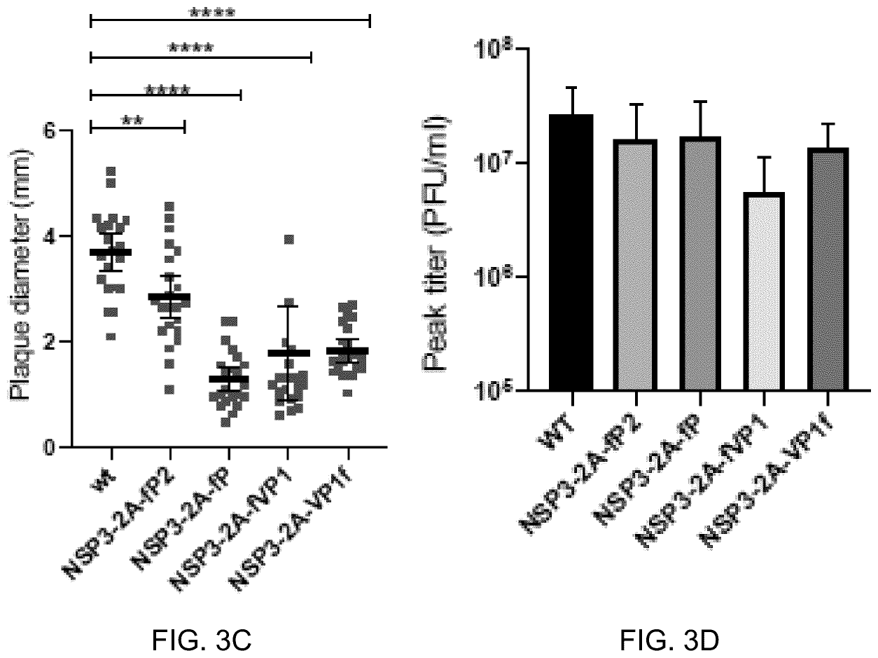

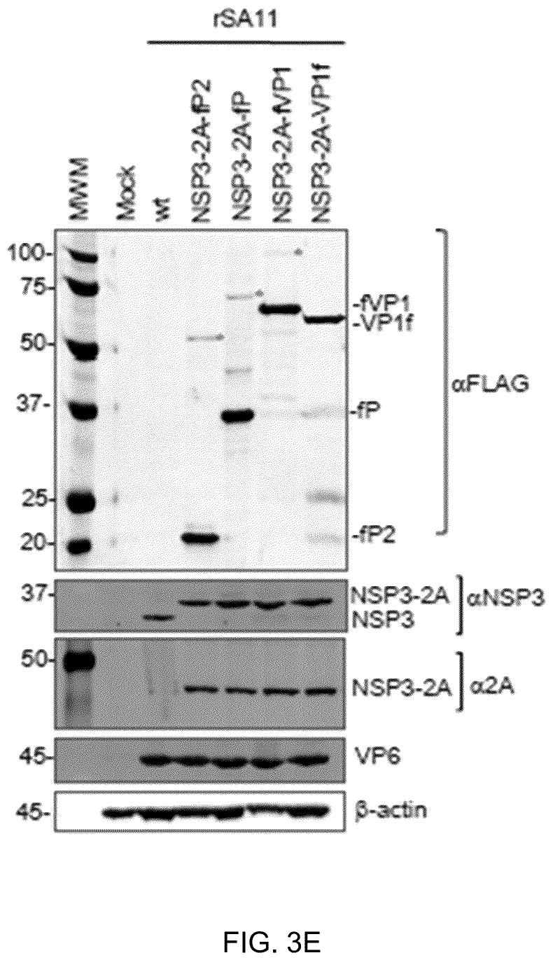

rSA11 viruses generated with modified pT7 NSP3-2A-NoV vectors expressing FLAG-tagged NoV proteins contained larger segment 7 dsRNAs than that of wildtype virus (rSA11/wt), based on RNA gel electrophoresis (FIG. 3A). Sequence analysis showed that segment 7 of the rSA11 viruses matched that of the pT7 NSP3-2A-NOV vectors. The introduction of FLAG-tagged NoV P2 and P into the genome segment 7 has increased the size to 1.7 kbp and 2.1 kbp respectively, that migrated on RNA gels between RV genome segments 4 (2.4 kbp) and 5 (1.6 kbp), as expected from their sizes (Table 2, FIG. 3A.). Similarly, the re-engineered segment 7 of virus isolates containing 1.7 kbp NoV VP1 protein sequences (NSP3-2A-fVP1 and NSP3-2A-VP1f) had a length of 2.9 kbp, resulting in a slower migratory position near RV genome segment 1. Thus, the insertion of 2A-fVP1 and VP1-f sequences into the segment 7 genome has increased the total genome size to 20.3 kbp, which is 9.5% more than the packaging capacity of the wt. virus. The longest foreign sequence introduced into segment 7 RNA of rSA11 previously was 3.3 kbp segment 7 dsRNA of rSA11/NSP3-fS1, that expressing SARS CoV-2 S1 protein.

Plaque analysis showed that the plaques formed by rSA11/wt virus were larger than plaques formed by rSA11/NSP3-2A-fP2, -fP, -fVP1, and -VP1f viruses (FIG. 3B., FIG. 3C.), consistent with the data reported in a previous study. Quantitation of the viral peak titers demonstrated that rSA11/NSP3-2A-fP2, -fP, -fVP1, and -VP1f viruses grew to similar titers in MA104 cells, ranging from 0.5×10⁷ to 2.6×10⁷ (FIG. 3D). The exact reason for the smaller plaque phenotypes and lower titers is unknown but may be possibly due to the longer elongation time required for the viral RNA polymerase to transcribe the modified segment 7 dsRNAs during viral replication or may be the longer time required for translating the segment 7 mRNA containing foreign protein sequences. Alternatively, it may reflect the complexity associated with packaging the largely modified dsRNA containing foreign sequences and assembly of the viral particles.

To determine the expression of NoV protein products, MA104 cells were infected with the rSA11 strains and the whole-cell lysates (WCL) were examined by immunoblot assay using FLAG antibody (FIG. 3E). Immunoblots probed with FLAG antibody showed that rSA11/NSP3-2A-fP2, -fP, -fVP1 and -VP1f viruses generated NoV proteins as major products having predicted protein sizes for a functional 2A element in the modified NSP3 ORF: -fP2 (18.6 kDa), -fP (37.7 kDa), -fVP1 (61.9 kDa) and -VP1f (60.1 kDa) (Table 2 and FIG. 3E). Assays with anti-NSP3 antibody and 2A element antibody identified 38 kD protein that corresponds to NSP3 (36 kDa) linked to remnant residues of P2A element (2 kDa). However, immunoblot assay performed with anti-FLAG antibody detected minor amounts of large fusion proteins which represented the read-through products of the NSP3-2A-NOV protein cassette; NSP3-2A-fP2, NSP3-2A-fP, and NSP3-2A-fVP1 (FIG. 3E, red asterisks). However, the read-through product was not detected for rSA11/NSP3-2A-VP1f virus, suggesting that direct fusion of downstream ORF to the 2A peptide would result in the efficient stop-restart activity of the 2A element (FIG. 3E).

Recovery and Characterization of rSA11 Viruses Modified to Express NoV Capsid Proteins Containing C-Terminal 6×His Tag.

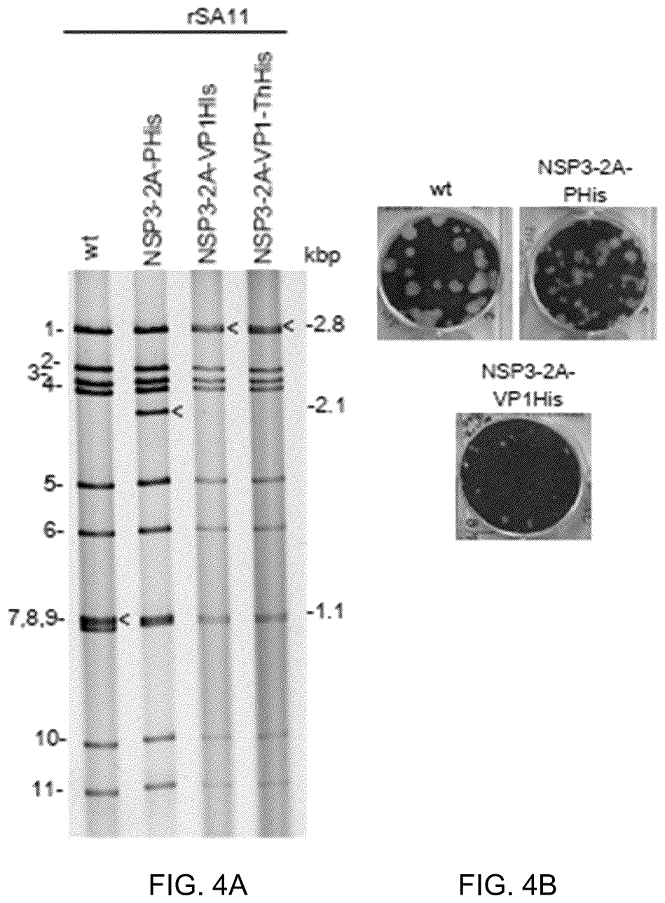

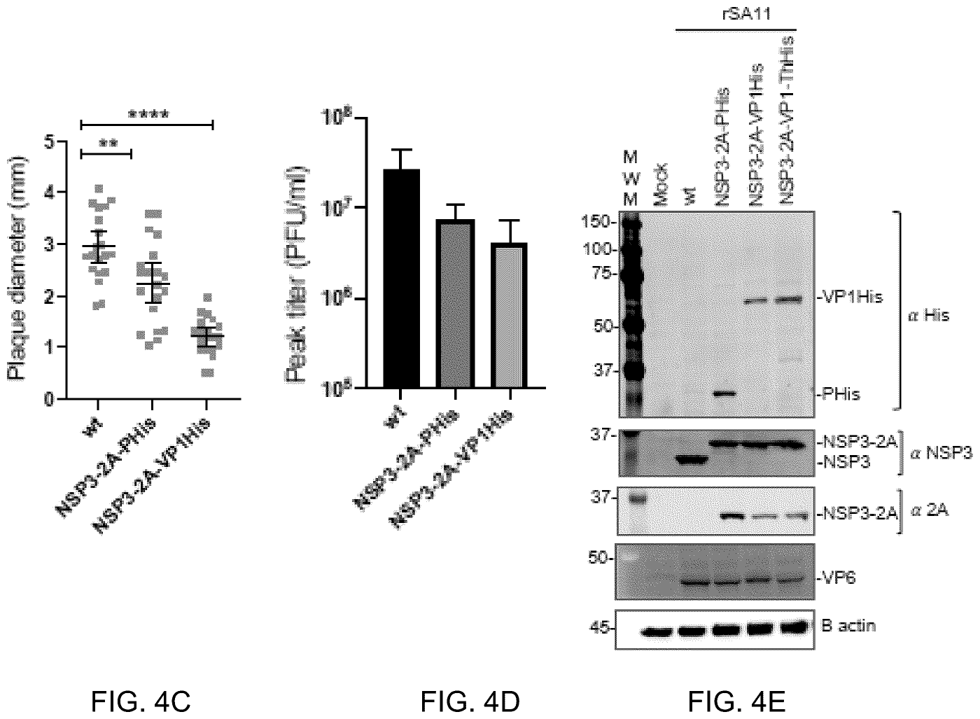

The genome segment 7 modification of viruses expressing 6×His-tagged NoV P and VP1 sequences had increased their lengths to 2.1 kbp and 2.8 kbp (FIG. 4A). As mentioned above, the plaque sizes of rSA11/NSP3-2A-PHis, -VP1 His, and -VP1 Th His were significantly smaller than rSA11/wt virus (FIG. 4B, FIG. 4C). Analysis of the peak viral titers for rSA11/NSP3-2A-PHis, -VP1His, -fVP1 Th His viruses showed that they grew to maximum titers that were up to 0.5 to 1 log lower than rSA11/wt (FIG. 4D).

Immunoblot assay performed with anti-6×His antibody showed that rSA11/NSP3-2A-P His, -VP1 His, -fVP1 Th His viruses generated P (35.8 kDa) and VP1 products (60 kDa) having sizes as predicted for a functional 2A element (FIG. 4E and Table 2). While, probing with anti-6×His antibody could not detect the presence of large fusion read-through products for these viruses, possibly because NoV ORF was inserted directly after the 2A element. Mirroring the results described above for viruses expressing FLAG-tagged NoV protein products, assay with anti-NSP3 antibody and 2A element antibody for rSA11/NSP3-2A-PHis, -VP1 His, -fVP1 Th His viruses identified NSP3-2A protein of 38 kD size (FIG. 4E).

Generation of rSA11 Virus Containing Rotarix Genome Segment 7 Expressing NoV P Protein.

To test the possibility of making human vaccine vector platforms, recombinant SA11 monoreassortant virus containing human vaccine strain Rotarix genome segment 7 (RIX 4414), modified to express NoV P protein was generated using the reverse genetics approach as described previously. The rSA11 virus containing RIX NSP3-2A-PHis was recovered by growing them on MA104 cells (African green monkey kidney cells), isolates were plaque purified and characterized as summarized in Table 2.

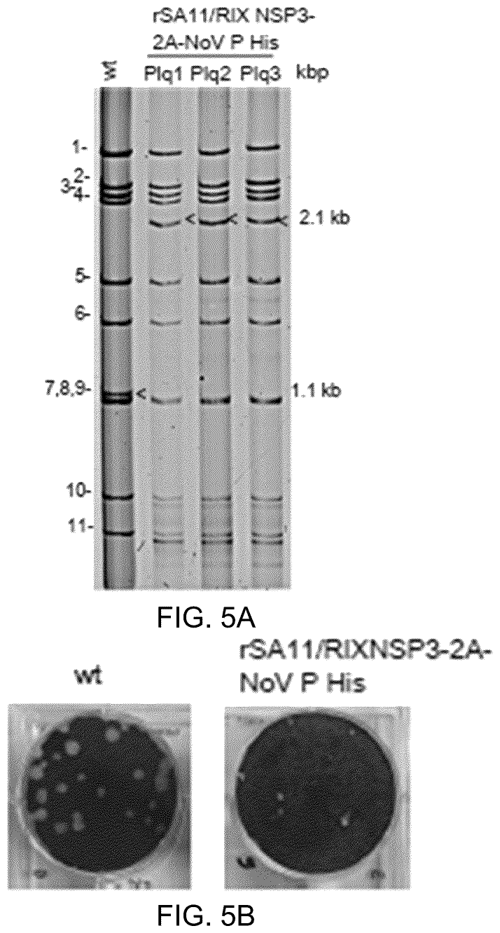

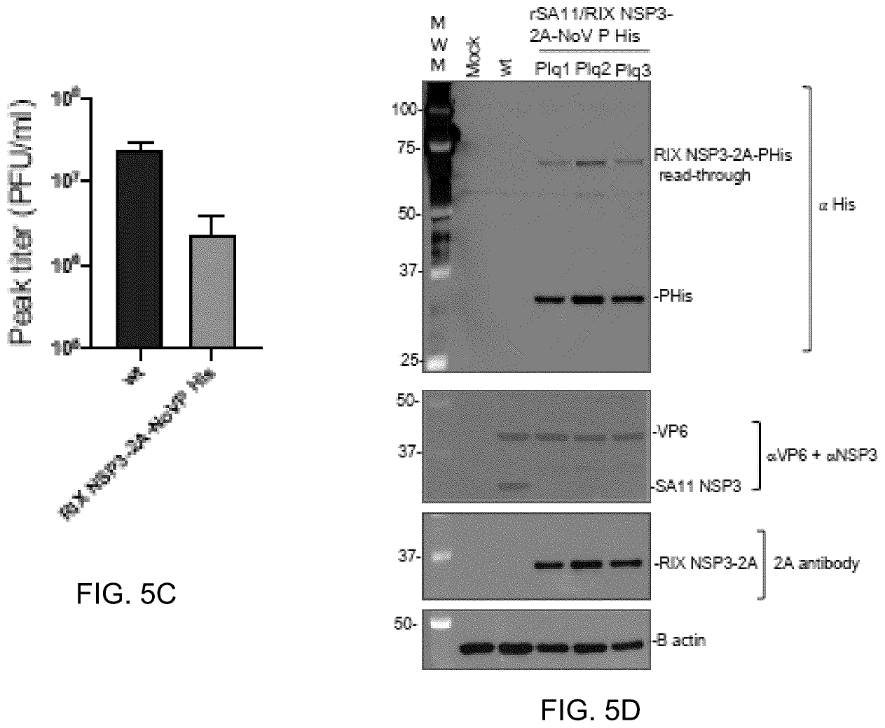

The rSA11/RIX NSP3-2A-PHis contained larger segment 7 dsRNA (2.1 kbp) than that of rSA11/wt (1.1 kbp) due to the introduction of 1 kb of 2A-NoV PHis sequence, based on RNA gel electrophoresis (FIG. 5A), and additional sequence introduction increased the total genome size to 19.6 kbp (Table 2). Plaque analysis showed that rSA11/RIX NSP3-2A-PHis formed smaller plaques than rSA11/wt virus (FIG. 5B) and they grew to a peak titer 1 log lower than rSA11/wt. (FIG. 5C).

To determine the expression of NoV P protein from the modified RIX NSP3 segment, MA104 cells were infected with three isolated plaques of rSA11/RIX NSP3-2A-PHis virus and the whole cells lysates were examined by immunoblot assay using anti-6×His antibody (FIG. 5D). Immunoblots results showed that rSA11/RIX NSP3-2A-PHis virus expressed 35.8 kDa NoV P protein as a major product and 74.2 kDa read-through product (red asterisks) as a minor product from the modified RIX NSP3 ORF (FIG. 5D and Table 2). Assay with anti-(SA11) NSP3 antibody identified only SA11 NSP3 protein of rSA11/wt (FIG. 5D); the antibody was not reactive with the NSP3 product of the RIX NSP3 ORF (FIG. 5D). As we do not have a specific antibody against RIX NSP3 protein, to test the expression of RIX NSP3 protein purposely, we re-probed the same blot with 2A antibody and identified robust expression of 38 kDa RIX NSP3 protein fused to 2 kDa 2A peptide (FIG. 5D). Overall, these results show that it is possible to generate SA11/RIX RV re-assortant strain that is modified to express immunogenic proteins of another enteric virus such as NoV.

Self-Assembly of VP1 Proteins Expressed from rSA11 Viruses to Form the Dimers

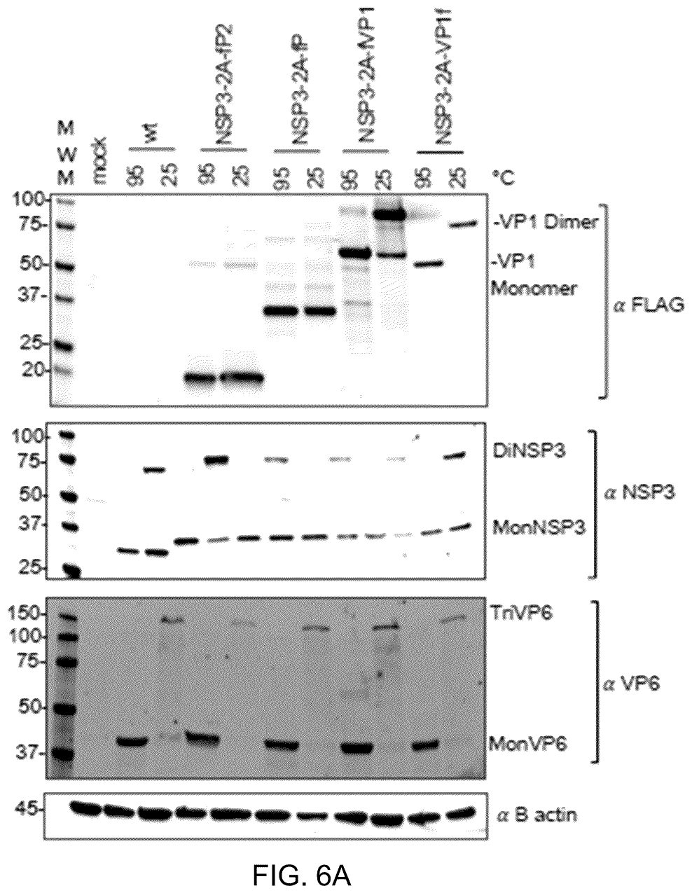

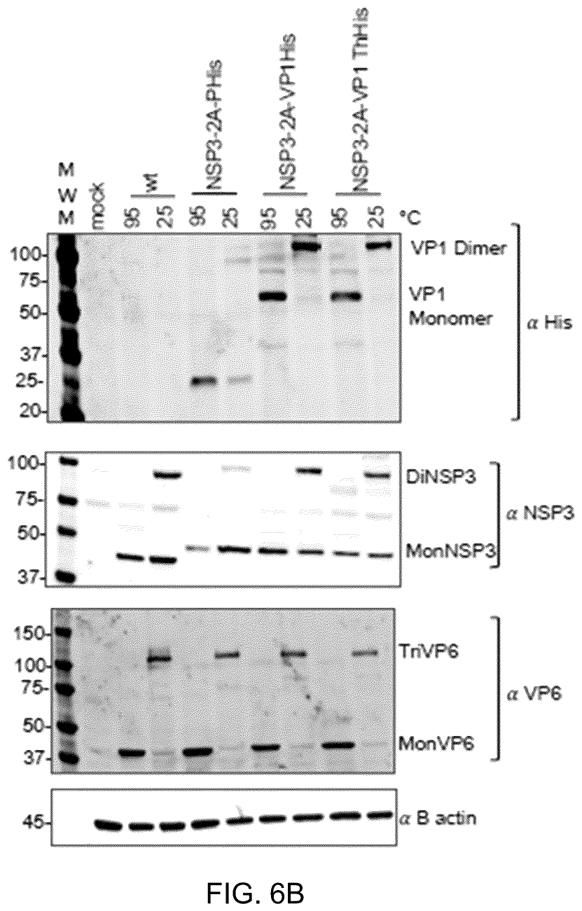

To examine whether the NoV protein products expressed from rSA11 viruses were able to form dimers in infected cells, lysates from rSA11/NSP3-2A-fP2, -fP, -fVP1, and -VP1f infected cells were treated with denaturing sample buffer at 25° C. Immunoblot assay with FLAG antibody showed that NoV P2 and P proteins did not form dimers, whereas both N terminal and C terminal FLAG-tagged VP1 proteins (fVP1 and VP1f) of rSA11/NSP3-2A-fVP1 and -VP1f lysates migrated as VP1 dimers (FIG. 6A, lanes 10 and 12), suggesting that VP1 dimer formation modulated by the intra-dimer interaction by the P domains are sustained in expressed VP1 proteins. Under the same electrophoretic conditions, both NSP3 and VP6 proteins from rSA11/wt and rSA11/NSP3-2A-fP2, -fP, -fVP1 and -VP1f viruses formed the dimers and trimers that were stable at 25° C. (FIG. 6A), consistent with the earlier reports. A similar result for VP1 dimerization was observed for His-tagged VP1 proteins (VP1His and VP1Th His) when the lysates from rSA11/NSP3-VP1 His and -VP1 Th His infected cells were probed with anti-6×His antibody (FIG. 6B). Mirroring the results indicated above, NSP3 and VP6 proteins formed stable dimers and trimers under the same electrophoretic conditions (FIG. 6B).

Folding of NoV VP1 Capsid Proteins into Native Structures

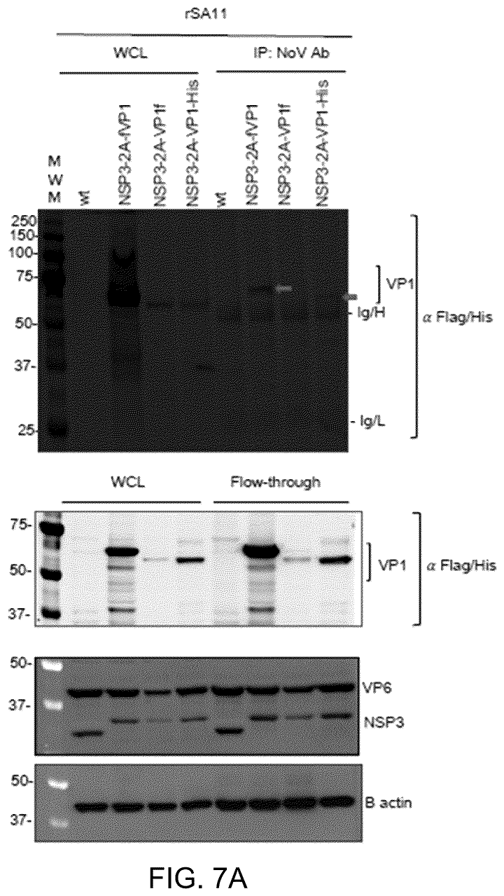

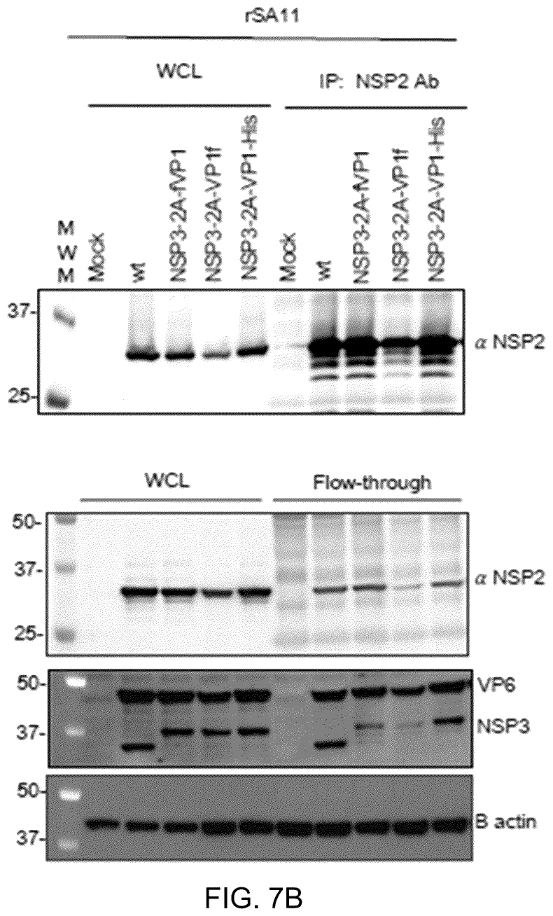

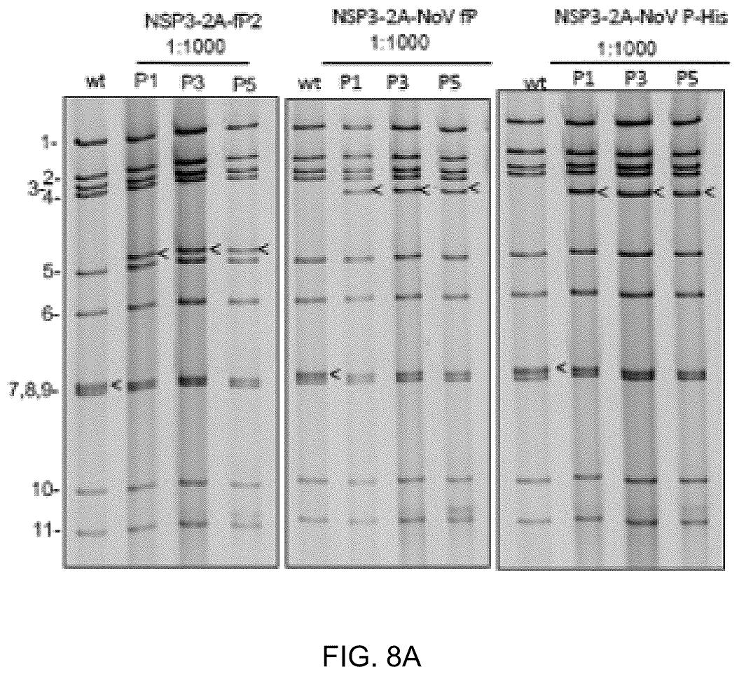

To gain insight into whether the VP1 products expressed from rSA11/NSP3-2A-VP1 viruses folded into native structures, lysates prepared from MA104 cells infected with rSA11/NSP3-2A-fVP1 and -VP1f, -VP1His viruses were probed by pulldown assay using an anti-NoV VP1 conformation-dependent neutralizing monoclonal antibody (NVB43.9, Absolute antibody). As shown in FIG. 7A (blue arrow), anti-NoV GII.4 NVB43.9 antibody immunoprecipitated both FLAG and His tagged NoV VP1 proteins (fVP1 and VP1His), indicating that at least some of the VP1 proteins expressed from rSA11 viruses folded in the correct conformation to include an authentic neutralizing epitope found in the NoV VP1 protein, which is capable of inducing a protective immunological response. Unlike the successful pull-down of fVP1 and VP1His with anti-NoV GII.4 antibody, it was not clear whether the antibody likewise immunoprecipitated the VP1f product of rSA11/NSP3-2A-VP1f. These results may relate to the lower signal intensity for 1×FLAG of VP1-f or the heavy chain, Ig/H of anti-NoV GII.4 antibody obscuring the closely migrating 60 kDa VP1f (FIG. 7A). The lysates were similarly analyzed using an NSP2-specific monoclonal antibody (FIG. 7B). A further question was whether the lower yield of immunoprecipitated VP1 protein is either due to the lower affinity of anti-NoV GII.4 NVB43.9 antibody or the elution condition. To answer this question, the presence of VP1, NSP3, and VP6 proteins in WCL and flow-through was analyzed by probing with FLAG, NSP3, and VP6 antibodies. The results showed that a major fraction of VP1 proteins remained in the flow-through, and only a small amount of VP1 protein was immunoprecipitated (FIG. 7A, lower panel). This was further validated by testing the amount of NSP2, NSP3, and VP6 proteins in WCL and flow-through of NSP2 immunoprecipitation samples, which showed that a major portion of the NSP2 proteins was successfully immunoprecipitated and recovered from the beads (FIG. 7B, lower panel). Taken together, the results suggest that at least some of the VP1 proteins expressed from rSA11 viruses folded in the correct conformation, and the low yield of VP1 immunoprecipitate might be due to the poor binding affinity of the anti-NoV GII.4 antibody.

Genetic Stability of rSA11 Strains Expressing NoV Proteins

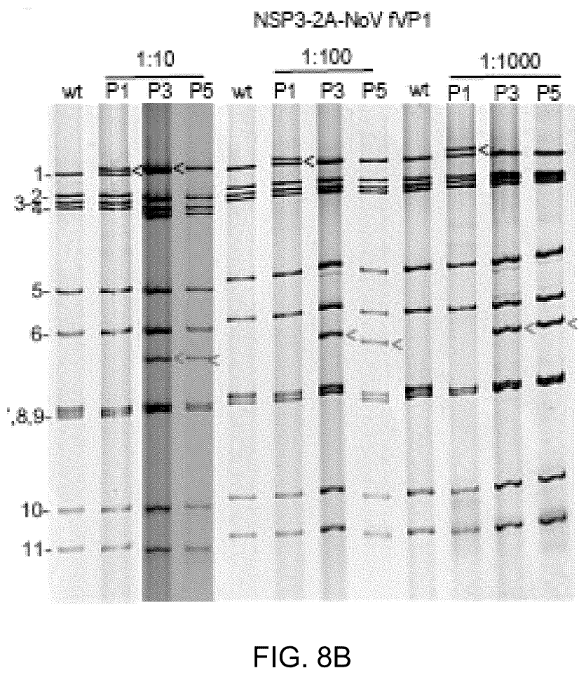

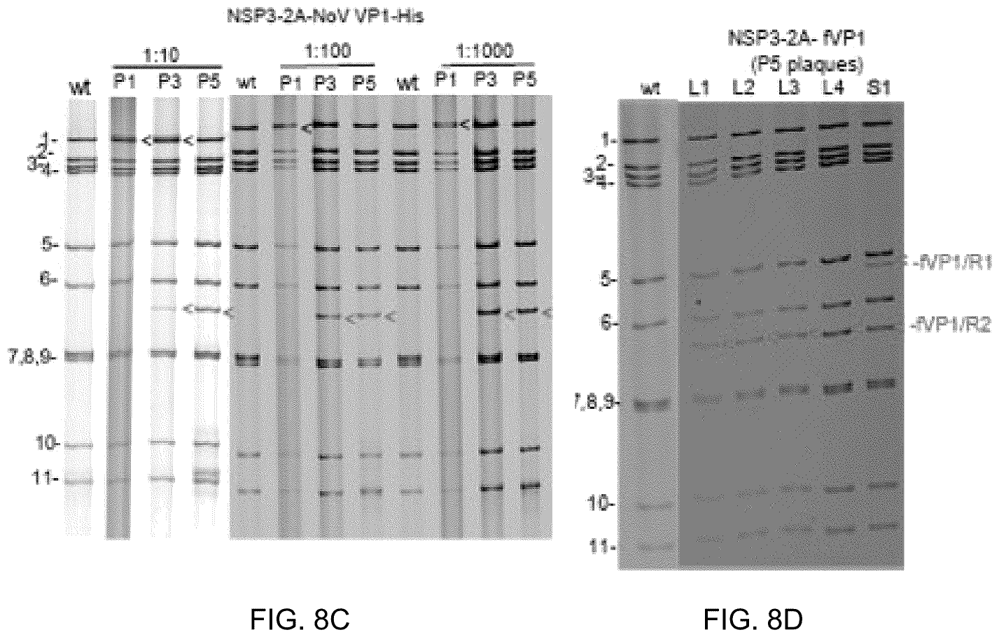

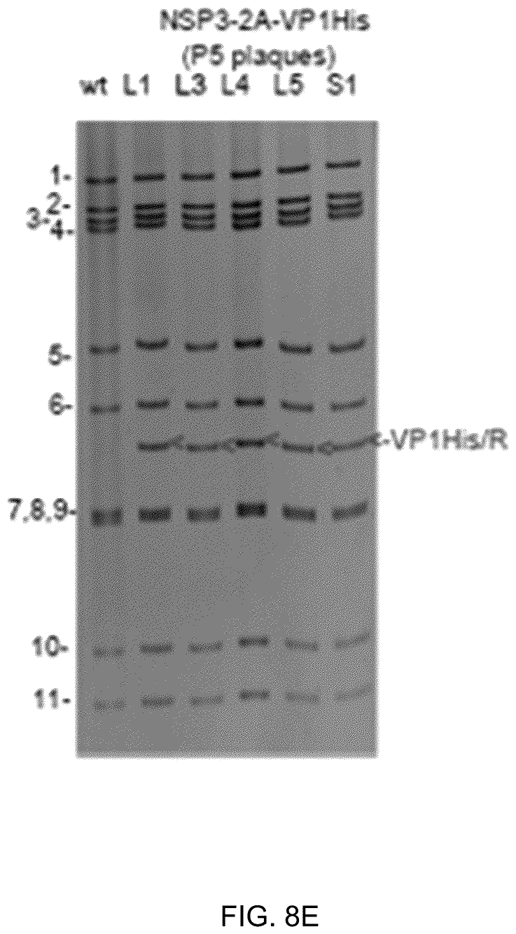

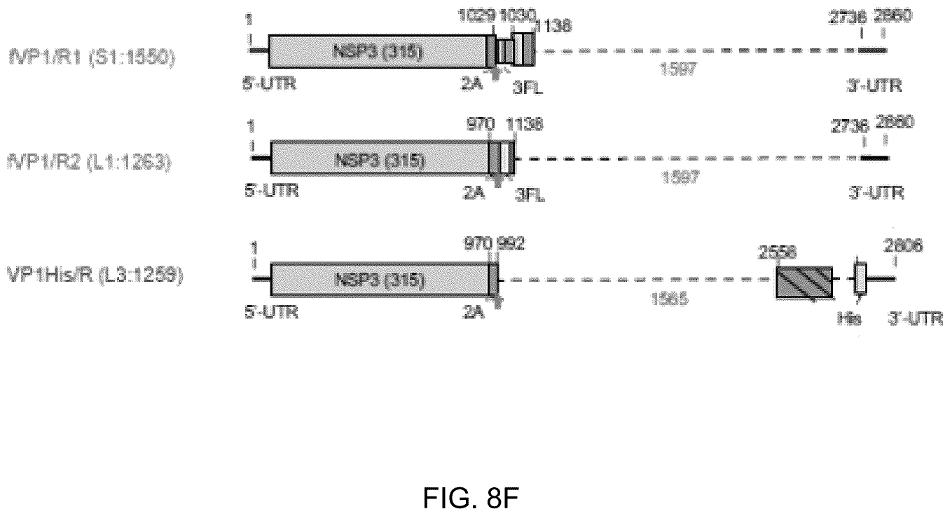

To analyze the genetic stability of rSA11 viruses expressing NoV capsid proteins, rSA11 viruses expressing both FLAG and His tagged proteins (rSA11/NSP3-2A-fP2, -fP, -PHis, -fVP1, and VP1-His) were subjected to 5 rounds of serial passage at three dilutions (1:10, 1:100 or 1:1000). Gel electrophoresis of the dsRNAs recovered from cells infected with rSA11/NSP3-2A-fP2, -fP, and fHis showed no changes in sizes of any of the genome segments including modified genome segment 7, over 5 rounds of serial passage (P1 to P5), indicating that the viruses carrying up to 1.1 kbp of foreign sequences were genetically stable (FIG. 8A), consistent with a previous study. In contrast, serial passage of rSA11/NSP3-2A-fVP1 and -VP1His showed evidence of genetic instability at all three dilutions (FIG. 8B and FIG. 8C). New genome segments were appearing by the third round of passage, that were smaller than original modified g7 segments, NSP3-2A-fVP1 and -VP1 His of 2.9 kbp size. With subsequent passages, a variant segment of size 1.2 kbp migrating below genome segment 6 became prominent, and 2.9 kbp segment 7 was not detectable by P5 generation, suggesting that high passage virus pools were dominated by variants derived from 2.9 kbp segment 7 RNA through sequence deletion. To test the possibility of internal sequence deletion, 5 variants were recovered from P5 virus pools by plaque isolation, four with a large (L) plaque phenotype and one with a small(S) plaque phenotype. Gel electrophoresis performed on dsRNAs extracted from isolated plaques showed that none contained original 2.9 kbp genome segment 7 RNA (FIG. 8D, FIG. 8E). Instead, L1-L4 variants from NSP3-2A-fVP1 P5 pool contained re-arranged (denoted by letter R), fVP1/R2 segment whereas S1 variant contained both-fVP1/R1 and -fVP1/R2 segments (FIG. 8D). Likewise, RNA gel electrophoresis on dsRNAs isolated from rSA11/NSP3-2A-VP1His P5 pool showed that both the large (L1, L3-5) and small (S1) plaque lysates contained only a single type of small variant segment, -VP1 His/R (FIG. 8E).

Sequence analysis of variant segments revealed that-fVP1/R1 and -fVP1/R2 were originated from 2.9 kbp segment 7 RNA, NSP3-2A-fVP1, and -VP1 His/R was resulting from large NSP3-2A-VP1 His segment (FIG. 8F). The -fVP1/R1 (1550 bp) and -fVP1/R2 (1,263 bp) variant segments retained the complete 5′-UTR and NSP3 ORF of segment 7, but contained sequence deletions of 1.6 kbp of NoV VP1 coding sequences and initial 7 bp in 3′-UTR. The sequence alignment of -fVP1/R1 and -fVP1/R2 demonstrated that R1 had a duplication of 287 bases from the C-terminus of NSP3 ORF and 2A peptide sequences and that -fVP1/R2 may have originated from the -fVP1/R1 variant segment (FIG. 8F, Table 3). Likewise, sequencing of -VP1 His/R showed that the dsRNA retained complete 5′-UTR, NSP3 ORF of segment 7 and 3′-UTR, but had 1.6 kbp sequence deletion of NoV VP1 coding sequence (FIG. 8F). The fact that all the five variants isolated from the P5 pool of NSP3-2A-fVP1 and NSP3-2A-VP1His, by plaque assay, contained small variant segments, -fVP1/R2 and -VP1 His/R respectively, suggested that the variants with smaller RNA may have a growth advantage over the variants with full-length or larger genome segments (FIG. 8D, FIG. 8E), consistent with a previous report. Although all three variants had deletions of NoV coding sequences, these variants contained complete ORF of NSP3 suggesting that NSP3 protein may be essential for viral replication. Further analysis of the total population of viral RNAs in all the passages by direct RNA sequencing may provide a better insight into the mechanism and diversity of deletions introduced into the modified genome segment 7 carrying longer foreign sequences (NSP3-2A-VP1)

### Sequence Deletions Due to Genetic Instability are not Limited to Inserted Foreign Sequences

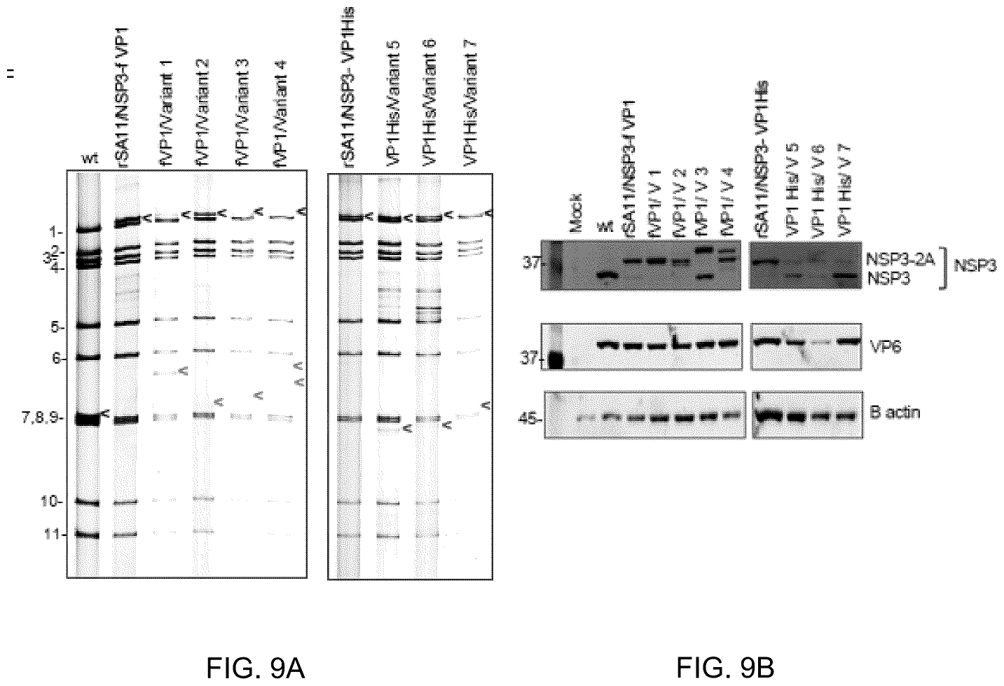

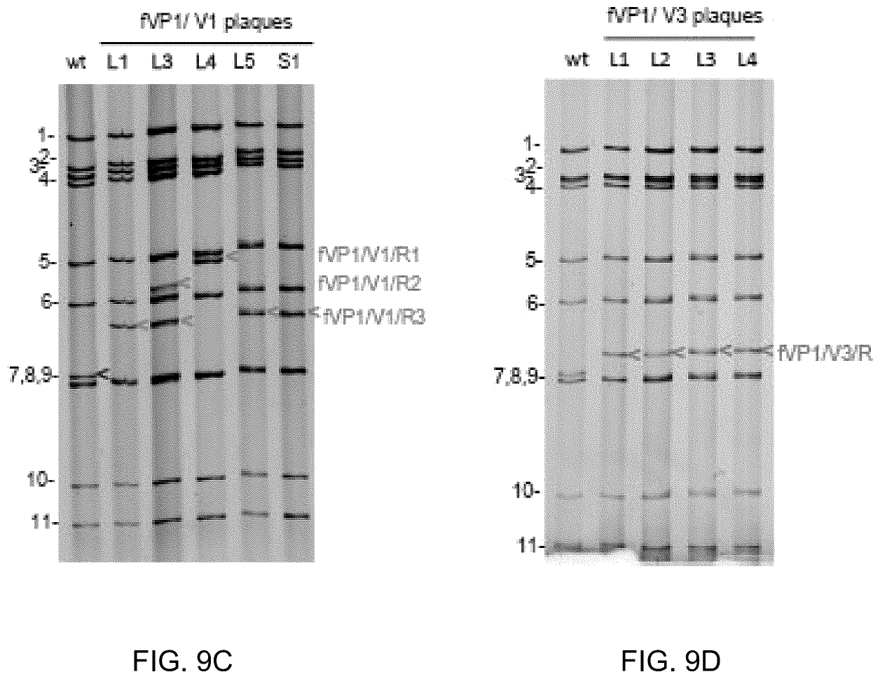

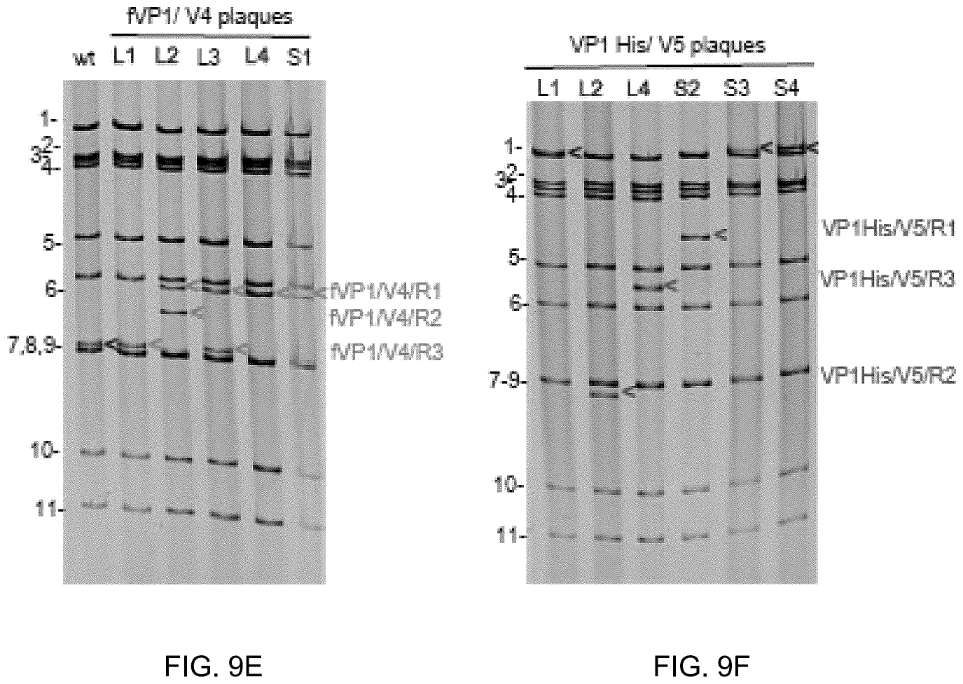

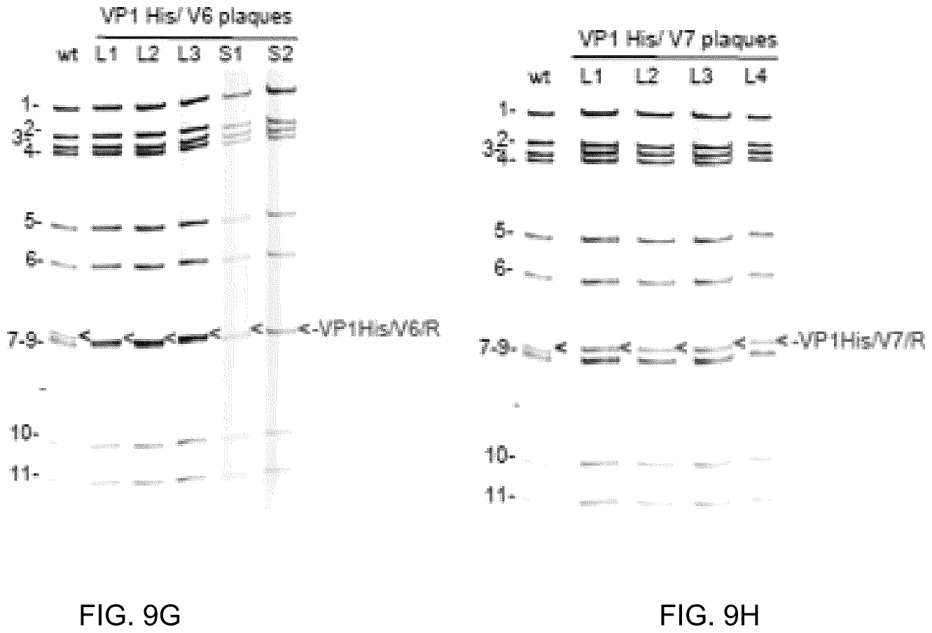

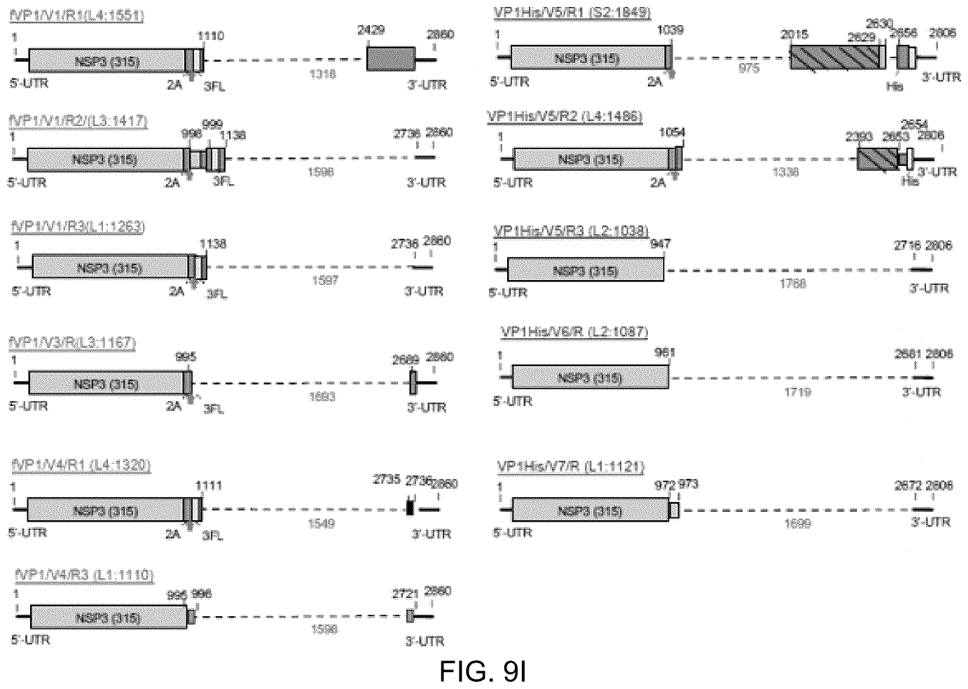

To gain a better understanding of the potential hotspot regions and the nature of genetic instability, rSA11/NSP3-2A-fVP1 and -VP1His viruses were amplified to larger volumes at a low MOI (multiplicity of infection). Gel electrophoretic analysis of extracted dsRNAs identified diverse variant pools (denoted as V) that contained different types of re-arranged genome segment 7, of varying sizes for rSA11/NSP3-2A-fVP1 virus (fVP1/V1-V4) and rSA11/NSP3-2A-VP1 His virus (VP1 His/V5-V7) (FIG. 9A, red and blue arrows). Immunoblot assay performed on lysates prepared from MA104 cells infected with variant pool viruses (fVP1/V1-V4 and VP1 His/V5-V7) detected two or more forms of NSP3 proteins when probed with NSP3 antibody. This suggests that variant genome segments expressed a smaller NSP3 protein, similar to wt. NSP3 protein, whereas the original genome segment 7 containing NSP3-2A-VP1 cassette expressed NSP3-2A protein, as expected (FIG. 9B). Occasionally, there were some modified NSP3 proteins, even larger than the expected NSP3-2A protein size, probably caused by the fusion of a few VP1 residues to NSP3-2A peptide following sequence deletion (FIG. 9B).

For further evaluation, four to five rSA11 isolates were recovered and characterized from each of the above variant pools. Sequencing of the segment 7 dsRNAs of each plaque-purified isolate from variant pools (V1 to V7) revealed the appearance of re-arranged genome segments (R), derived from 2.9 kbp segment 7 RNA of rSA11/NSP3-2A-fVP1 and -VP1 His viruses (FIG. 9C. to FIG. 9H.) The R1, R2, and R3 RNAs generated from fVP1/V1 pool retained 5′-UTRs and NSP3 ORF, but contained sequence deletions of 1.3 (R1), 1.6 (R2 and R3) kbp of the VP1 coding sequence, and either a 7 bp deletion or a 9 bp duplication in 3′-UTRs (FIG. 9I). Interestingly, the R2 isolate from fVP1/V1 pool showed a 154 bp duplication, identical to the last few amino acids present in NSP3 ORF and few residues of 2A element, inserted in the middle of 2A peptide (FIG. 9C, FIG. 9I and Table 3). Plaque isolates from variant 2 pool showed only one kind of re-arranged genome segment 7 (fVP1/V3/R) (FIG. 9D). The fVP1/V3/R segment contained the complete 5′- and 3′-UTRs and NSP3 ORF of segment 7 and lacked the entire 3× FLAG sequence. A portion of the 2A sequence was also missing along with all but the last 40 bases of the VP1 sequence (FIG. 9I). On the contrary, RNA gel electrophoresis of dsRNAs of plaques isolated from fVP1/V4 pool and VP1 His/V5 pool identified various R segments, one of them (VP1 His/V5/R3) had a smaller genome segment 7 (1038 bp) than rSA11/wt (1104 bp) (FIG. 9F, lane 2). The smaller size of VP1 His/V5/R3 stemmed from a larger deletion of 1.8 kbp of inserted foreign sequences including 23 bp of NSP3 ORF and 41 bp of 3′-UTR regions (FIG. 9I and Table 3). Similarly, VP1 His/V6/R isolate contained a smaller genome segment 7 (1087 bp) (FIG. 9G, lanes 2-5), due to the deletion of 9 bp from NSP3 ORF and 6 bp from 3′-UTR along with the entire 2A-VP1-His foreign sequences (FIG. 9I). Furthermore, the variant plaques (VP1 His/V7/R) isolated from the VP1 His/V7 pool identified a re-arranged genome segment 7 that contained the complete 5′- and 3′-UTRs and NSP3 ORF of segment 7, but contained a 14 bp duplication of NSP3 sequence, and deletion of 1.7 kb of VP1-His coding sequences (FIG. 9H, FIG. 9I). Taken together, these sequencing results suggest that RV strains are still able to replicate efficiently even if they lost some nucleotides in 3′ end of NSP3 ORF and the first few located in 3′ UTR region (FIG. 9F, lane 2 and 9 FIG. 9G, lanes 2-5). Thus, the rSA11 strains modified to express larger foreign sequences such as NoV VP1 become genetically unstable on subsequent passages, and the instability accounts for the deletion of inserted foreign sequences or NSP3 sequence.

The Density of rSA11 Virus Particles Containing NoV Capsid Sequences

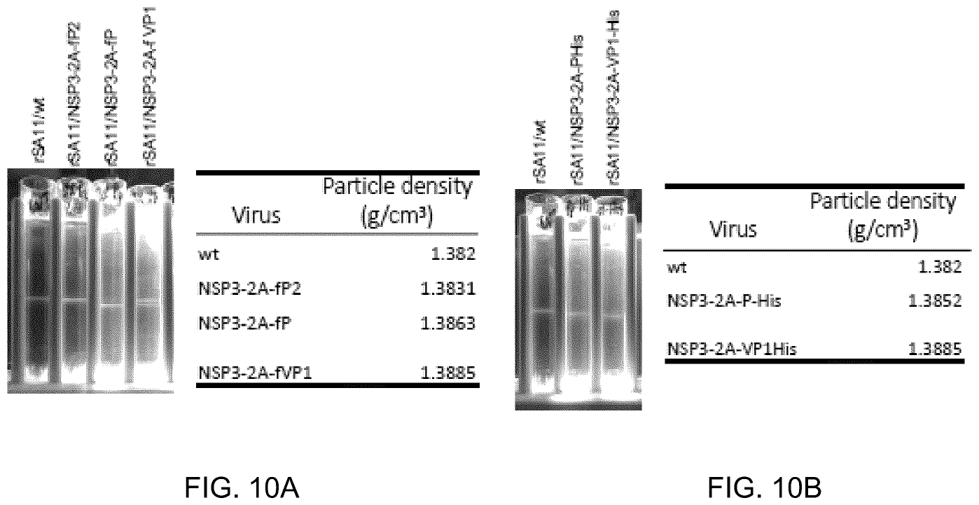

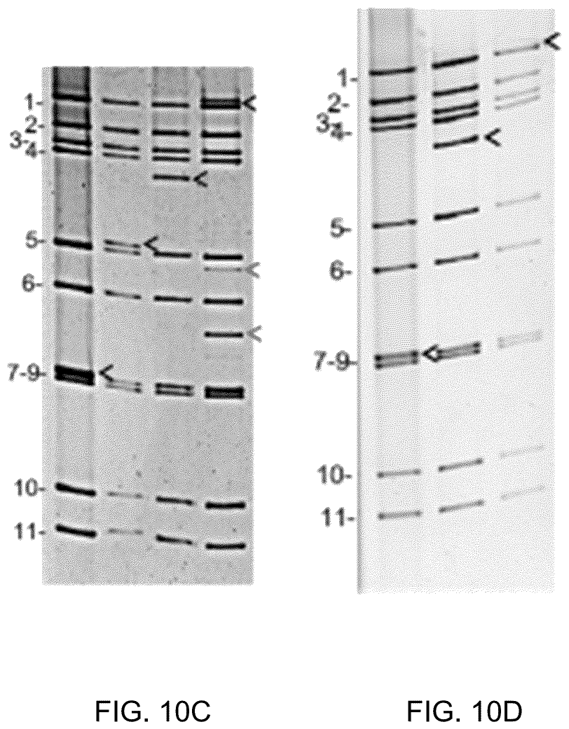

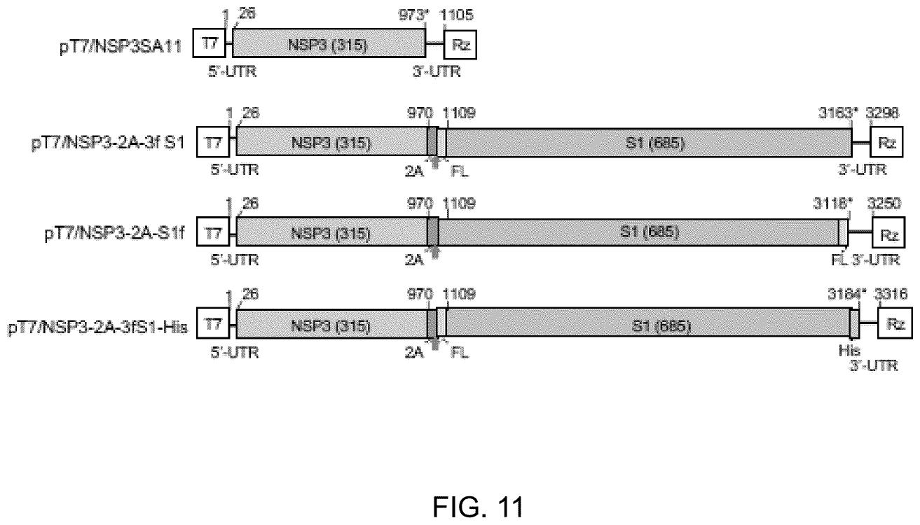

The rSA11 viruses re-engineered to express NoV capsid proteins from the modified segment 7, contained large viral genomes of size 0.5 to 2.1 kbp greater than that of SA11/wt. RNA gel electrophoresis showed that rSA11/NSP3-2A modified viruses are packaged efficiently and contain a complete constellation of all eleven (11) genome segments (FIGS. 3A, 4A, and 5A); and packaging of additional sequences within the core should change the density of viral particles. To explore this possibility, rSA11/wt (genome size: 18.6 kbp), rSA11/NSP3-2A-fP2 (19.1 kbp), rSA11/NSP3-2A-fP (19.6 kbp), rSA11/NSP3-2A-fVP1 (20.3 kbp), rSA11/NSP3-2A-PHis (19.6 kbp) and rSA11/NSP3-2A-VP1 His (20.2 kbp) were amplified on MA104 cell monolayers. The double-layered particles (DLPs) were prepared from the infected-cell lysates by converting RV triple-layered particles after treating with EDTA. The DLPs were centrifuged to equilibrium on CsCl gradients and the density of the DLP bands was determined by refractometry (FIG. 10A, FIG. 10B). The analysis indicated that the densities of rSA11/NSP3-2A-fP2 DLPs (1.3831 g/cm³), rSA11/NSP3-2A-fP DLPs (1.3863 g/cm³) and rSA11/NSP3-2A-fVP1 DLPs (1.3885 g/cm³) were greater than SA11/wt DLPs (1.382 g/cm³) (FIG. 10A). Similarly, the densities of rSA11/NSP3-2A-PHis (1.3852 g/cm³) and rSA11/NSP3-2A-VP1His (1.3885 g/cm³) were greater than SA11/wt DLPs (1.38 g/cm³) (FIG. 10B). Analysis of the dsRNAs extracted from banded DLPs by gel electrophoresis confirmed that they contained the expected constellation of the eleven genome segments (FIG. 10C, FIG. 10D). However, the banded DLPs from rSA11/NSP3-2A-fVP1 showed two bands in CsCl, which is probably due to the fact that the banded DLPs contained diverse virus isolates including some variants (FIG. 10A). This was further confirmed by gel electrophoresis of the dsRNAs extracted from banded-fVP1 DLPs, that identified three kinds of genome segment 7, of sizes 2.9 kbp (black arrow), 1.55 kbp, and 1.3 kbp (red arrows) (FIG. 10C, lane 4), exactly matching to that shown in FIG. 8D, lane 6. These data indicated that rSA11/NSP3-2A-NOV viruses that carry an extra 1.8 kbp of heterologous sequences hold complete genome constellations even though their genome segment 7 size was significantly greater than that of wildtype SA11 virus. Indeed, the 20.2 kbp rSA11/NSP3-2A-fVP1 and VP1-His genome are 9.1% greater in size than the 18.6 kbp rSA11/wt genome (Table 1). These results show the RV core has space to accommodate large amounts of additional foreign (heterologous) nucleic acid sequences that can encode various proteins for example consistent with prior reports. The largest foreign sequence inserted into rSA11/genome segment 7 so far is 2.6 kbp (data not shown), but the maximum packaging capacity of the core remains to be determined. These findings are consistent with earlier studies showing that the density of RV variants with naturally occurring sequence duplications or inserted foreign sequences was greater than that of wildtype RV.

### DISCUSSION

As demonstrated herein it is possible to generate rRVs that express portions of NoV capsid proteins, as separate proteins, through the modification of genome segment 7. These results indicate that RVs can be used as potential vaccine expression vectors. These result provide for example a method for generating combined oral live attenuated RV-NoV vaccine capable of preventing both RV and NoV mediated AGE in children. In recent years, although some NoV vaccine preparations have been tested in adult trials, it is highly required to explore the vaccine candidates for use in infants and young children. In this study, we generated a panel of rRV expressing NoV capsid domains and tested the expression of proteins, and genetic stability. All recombinant RVs grew to high titers in cell culture and the NoV proteins were highly expressed, making it an excellent expression platform for use in children as there is no pre-existing immunity against RV or NoV in them. Moreover, RV has an extremely high level of antigen expression while using as a vaccine, enabling the generation of a strong immune response, therefore it can act as an adjuvant, increasing the immune response to NoV antigens. Furthermore, RV genome can accommodate up to 1.3 kbp of additional sequences without genetic instability, allowing the accommodation of multiple foreign genes, developing as a multivalent vaccine vector. This raises the possibility of generating a multivalent vaccine by re-engineering RV to express immunodominant regions of other viruses such as SARS (severe acute respiratory syndrome caused by a SARS-associated coronavirus) including CoV-2 (also known as COVID-19) RBD (receptor binding domain) and NoV P2 for infants and children (RV-SARS-CoV-2-NoV vaccine), by, for example, replacing the current RV vaccines.

The analysis indicated that recombinant RVs expressing NoV capsid proteins grew to high titer (0.5×10⁷ to 2.6×10⁷ PFU/ml) in cell culture, consistent with a previous report and this high titer would make the vaccine production economically more feasible. The stop-restart activity of the 2A element showed some variations in rSA11 viruses. The viruses modified to express N terminal FLAG-tagged NoV proteins and RIX segment 7 modified to express NoV P-His protein produced a minor amount of NSP3-2A-read through products (FIGS. 3E and 5D, red asterisks). While the exact reason for this need not be known to practice the invention, is is probably because they all contained a flexible Ala-Ser linker in front of the starting codon of downstream NoV protein. Such a fusion read-through product was not found in the case of viruses described in FIG. 4E, and the sequence analysis showed that those viruses do not have the Ala-Ser linker but contained a direct fusion of NSP3-2A sequences to the start codon of downstream NoV protein (FIG. 3E, lane 6 and FIG. 4E).

The data suggest that VP1 proteins expressed from the modified genome segment 7 forms dimers and are capable of folding in the correct conformation. Also, the NSP3 protein of recombinant RVs expressing NoV proteins is functional, retaining the ability to form dimers, and is able to express the complete complement of all viral proteins.

The upper limit on the amount of heterologous sequence that can be accommodated into the RV genome has not been determined at present, however naturally occurring RV strains having natural sequence duplication contained an additional 0.9 kbp of segment 7 sequences, increasing its size to 2.0 kbp (56). In this study, generation of rSA11/NSP3-2A-fVP1 virus resulted in accommodating 1.8 kbp of foreign sequence, sufficient to encode NoV VP1 protein, and increasing the total genome size to 20.3 kbp. But, this is not the largest recombinant RV made to date, dsRNA that can accommodate 2.2 kbp of the foreign sequence encoding SARS-CoV-2 S1 protein was previously made. The data demonstrate that RVs carrying large heterologous sequences e.g., 1.8 kbp NoV VP1, have smaller plaque phenotypes and are genetically unstable resulting in the development of new variants over subsequent amplification. The exact reason for the smaller plaque phenotype and genetic instability is unknown, but under investigation (data not shown). A proposed hypothesis on sequence rearrangement suggested that the viral RNA polymerase could have interrupted the RNA synthesis, either during transcription or replication, fall back on its-own template to re-initiate RNA synthesis (57, 58). However, RVs carrying up to 1.3 kbp are found genetically stable over 5 rounds of serial passage (43) and, thus, can be developed into vaccine platforms. The coding capacity provided by 1.3 kbp of the foreign sequence is sufficient to make RVs expressing NoV P proteins (1.1 kbp) along with some further modification such as fusion of a secretory signal or Fc binding protein. This kind of protein modifications, such as Fc-immunoglobulin G1 (Fc-IgG1), or ligands for cell surface receptors can specifically target the expressed protein to a specific cell type (antigen-presenting cells or T cells). Further modification of the NoV P protein with carrier moieties, such as a cell-penetrating peptide or a secretion peptide (e.g., Interleukin-2 secretion peptide), can achieve efficient transport of the expressed protein across the cell membranes.

The results suggested that it is possible to re-engineer human vaccine strain genome segment 7 (RIX 4414) to express NoV protein, providing a way for developing human RV-NoV vaccine strain for use in children. Furthermore, a modified RV reverse genetics system capable of generating recombinant human RV strains, present in the current RV vaccines needs to be developed. Such human rRV vaccine strain expressing NoV proteins are likely to be tested to gain insight into the production of neutralizing antibodies in the immunized animals. The described RV systems can be used as effective vector systems, for developing combined vaccines that can protect against multiple diseases, or the same disease caused by two or more variants of the same pathogen, for example two or more diseases caused by two or more viruses or two or more variants of the same virus.

### Example 2—Recombinant Rotaviruses (RVs) Expressing Functional Glycoproteins

Earlier studies investigated the possibility of making recombinant RVs that expressed portions of the spike(S) protein of SARS-CoV-2 (Philip and Patton, 2021). In this work, rSA11 viruses with segment 7 modifications were recovered that expressed the N-terminal domain (NTD), the receptor binding domain (RBD), and the core domain (CR) of the S protein (Duan et al., 2020, Huang et al., 2020). A similar segment 7 modification was used to make a recombinant virus (rSA11/NSP3-2A-fS1) containing the complete coding sequence of the SARS-CoV-2 S1 protein, a cleavage fragment of the S protein that includes both the NTD and RBD and is a primary target of neutralizing antibodies produced during SARS-CoV-2 infection (Brouwer et al., 2020, Liu et al., 2020, Rogers et al., 2020, Zost et al., 2020, Xin et al, 2021). The open reading frame (ORF) in the modified segment 7 RNA of the rSA11/NSP3-2A-fS1 virus included the coding cassette NSP3-2A-3×FLAG-S1. Through the action of the 2A translation element, the segment 7 RNA of the virus was expected to generate two products: NSP3 fused to a 2A peptide (NSP3-2A) and 3×FLAG-tagged S1 (fS1). In the NSP3-2A-3×FLAG-S1 cassette, a 3×FLAG tag was positioned immediately upstream of the S1 signal peptide, an element critical for synthesis of glycosylated S products (Casalino et al., 2020). Immunoblot analysis of the products made by rSA11/NSP3-2A-fS1 indicated the while the virus efficiently made NSP3-2A, it was not efficient in generating the expected fS1 product, possibly due to instability or degradation of the S1 product, or impact of the FLAG tag on the function of signal peptide (Philip and Patton, 2021). The work described here compares the S1 products made by the rSA11/NSP3-2A-fS1 virus to the products made by newly designed rSA11 viruses encoding S1 proteins differing in the nature of their terminal peptide tags. The results showed that the newly designed rSA11 viruses efficiently expressed S1 proteins and that the S1 proteins were glycosylated and biologically functional, as measured by their affinity for the extracellular domain of the ACE2 receptor (Medina-Enriquez, 2020). This demonstrates that the recombinant RVs can be used as expression vectors of glycosylated foreign proteins.

### Materials and Methods

### Cell Culture

Embryonic monkey kidney cells (MA104) were grown in Dulbecco's modified Eagle's medium (DMEM) containing 4.5 g/L glucose (Lonza 12-640F or Corning 15-107-CV), 1% penicillin-streptomycin [Corning]), and 5% fetal bovine serum (FBS, Gibco) (Arnold et al, 2009). Baby hamster kidney cells constitutively expressing T7 RNA polymerase (BHK-T7 cells) were kindly provided by Drs. Ulla Buchholz and Peter Collins, Laboratory of Infectious Diseases, NIAID, NIH. BHK-T7 cells were grown in Glasgow complete medium (GMEM, Lonza) supplemented with 10% tryptone-peptide broth (Gibco), 1% penicillin-streptomycin, 2% non-essential amino acids (Gibco), 1% glutamine, and 5% heat-inactivated FBS (Philip et al., 2020). Medium used to cultivate BHK-T7 cells was supplemented with 2% G418 (Geneticin, ThermoFisher) every other passage.

### Plasmids

Plasmids used in generating rSA11 viruses were obtained from Addgene [https://www.addgene.org/Takeshi_Kobayashi/] and included pT7/VP1SA11, pT7/VP2SA11, pT7/VP3SA11, pT7/VP4SA11, pT7/VP6SA11, pT7/VP7SA11, pT7/NSP1SA11, pT7/NSP2SA11, pT7/NSP3SA11, pT7/NSP4SA11, and pT7/NSP5SA11. The plasmids pCMV-NP868R, pT7/NSP3-P2A-fUnaG, and pTWIST/COVID19spike were derived as described earlier (Philip et al, 2019; Philip and Patton, 2020, 2021). The plasmid pT7/NSP3-2A-3fS1 was generated as described by Philip and Patton (2021) and contains a full-length cDNA of the SARS-CoV-2 spike S1 open reading frame (ORF) (GenBank MN908947.3). The plasmids pT7/NSP3-2A-SIF and pT7/NSP3-2A-3fS1-His are the same as pT7/NSP3-2A-3fS1, containing the same S1 ORFs, but differ in sequences for peptide tags surrounding their S1 ORFs.

The pT7/NSP3-2A-S1f plasmid was constructed using a Takara In-Fusion cloning kit, which combined the vector backbone (pT7/NSP3-P2A region) of pT7/NSP3-P2A-fUnaG (primer pair for amplification: SEQ ID NO: 72 TGACCATTTTGATACATGTTGAACAATCAAATACAG and SEQ ID NO: 73, AGGACCGGGGTTTTCTTCCAC) with the S1 ORF insert of pTWIST/COVID19spike (primer pair: SEQ ID NO: 74. GAAAACCCCGGTCCTGTGTTTGTTTTTCTTGTTTTATTGCCACTAGTCT and SEQ ID NO: 75. GTATCAAAATGGTCACTTGTCATCGTCATCCTTGTAATCACGTGCCCGCCG). Primers were designed to introduce a 1×FLAG tag as the C-terminus of the encoded S1 protein. The pT7/NSP3-2A-3fS1-His plasmid was produced by inserting a sequence encoding a 6×His tag at 3′-end of the S1 ORF in pT7/NSP3-2A-3fS using an In-Fusion cloning kit. This was accomplished by amplifying pT7/NSP3-2A-3fS with the primer pair: SEQ ID NO: 76. ACCACCACCACCACCACTGACCATTTTGATACATGTTGAACA and SEQ ID NO: 77. GGTGGTGGTGGTGGTGACGTGCCCGCCGAGGAGA. Transfection quality plasmids were prepared using Qiagen plasmid purification kits. Primers were obtained from Eurofins Scientific and plasmid sequences were verified by Eurofins Genomics.

### Recombinant Viruses.

Detailed procedures for generating and recovering recombinant rSA11 have been published before (Philip et al., 2020; Philip and Patton, 2021). Briefly, BHK-T7 cells were transfected with SA11 pT7 plasmids and pCMV-NP868R using Mirus TransIT-LT1 transfection reagent. pT7/NSP2SA11 and pT7/NSP5SA11 were included in transfection mixtures at levels 3-fold higher than the other plasmids. As necessary, the pT7/NSP3SA11 plasmid was replaced with pT7/NSP3-2A-3fS, pT7/NSP3-2A-SIF or pT7/NSP3-2A-3fS1-His. The transfected cells BHK-T7 cells were overseeded with MA104 cells at 2 days post infection, and the growth medium was adjusted to a final concentration of 0.5 mg/ml trypsin (porcine Type IX pancreatic trypsin, Sigma Aldrich). Once complete cytopathic effects (CPE) was observed, cells in the media overlay were subject to three rounds of free thaw and the lysate clarified by low speed centrifugation. Virus in lysates were recovered by plaque isolation and amplified by one round of growth on MA104 cells (Philip et al., 2020). Viral dsRNAs were recovered by Trizol (Thermo Fisher) extraction (Philip et al, 2020), resolved by polyacrylamide gel electrophoresis, and detected by straining with ethidium bromide. cDNAs were generated from dsRNAs using a Superscript III One-Step RT-PCR Platinum Taq kit (Thermo Fisher) and appropriate segment 7 (NSP3) primers and sequenced by Eurofins Genomics.

### Immunoblot Analysis.

Proteins present in MA104 cell lysates were detected by immunoblot assay following previously described procedures (Philip et al., 2020; Philip and Patton, 2021). Cells were mock infected or infected with 5 plaque forming units (PFU) of recombinant virus, collected at 9 h.p.i., and lysed by resuspending in immunoprecipitation (IP) lysis buffer (300 mM NaCl, 100 mM Tris-HCl, pH 7.4, 2% Triton X-100) containing ethylenediaminetetraacetic acid (EDTA)-free protease inhibitor cocktail (Roche complete, Sigma Aldrich)]. Proteins were resolved by electrophoresis on 10% polyacrylamide (SDS) gels and transferred to nitrocellulose membranes using a Bio-Rad Trans-Blot Turbo Transfer System. Membranes were blocked with phosphate-buffered saline containing 5% non-fat dry milk and probed with rabbit polyclonal SARS-CoV-2 S1 antibody (A20136, ABclonal, 1:1000 dilution), guinea pig polyclonal NSP3 (NIH Lot 55068, 1:2000 dilution) or VP6 (NIH Lot 53963, 1:2000) antisera, mouse monoclonal FLAG M2 (F1804, Sigma-Aldrich, 1:2000) or anti-6×His antibody (MCA1396, Bio-Rad, 1:1000), or rabbit monoclonal b-actin antibody (D6A8, Cell Signaling Technology, 1:1000). In some cases, blots were re-probed with a different antibody following treatment with WesternSure ECL stripping buffer (LI-COR Biosciences). Primary antibodies were detected using 1:10,000 dilutions of horseradish peroxidase (HRP)-conjugated secondary antibodies: horse anti-mouse IgG (Cell Signal Technology), goat anti-guinea pig IgG [Kirkegaard & Perry Laboratories (KPL)], or goat anti-rabbit IgG (Cell Signaling Technology). Signals were developed using Clarity Western ECL Substrate (Bio-Rad) and detected using a Bio-Rad ChemiDoc imaging system.

### Endoglycosidase H (Endo H) Assay.

MA104 cell monolayers in 6-well plates were mock-infected or infected with rSA11 viruses (5 PFU per cell; PFU=plaque forming unit). At 9 h. p. i., cell monolayers were washed and scraped into phosphate-buffered saline (PBS), pelleted by low-speed centrifugation, and resuspended in 250 ml per well of IP lysis buffer. The presence of glycosylated proteins in the cell lysates were determined using a Promega Endoglycosidase H assay system (V4871). Briefly, 27 ml samples of cell lysates were combined with 3 ml of 10× Denaturing Solution, heated to 95° C. for 5 min, and cooled to room temperature. The heated-treated lysates were mixed with 3 ml of nuclease-free water, 4 ml of 10× Endo H Reaction Buffer and 3 ml of Endo H enzyme, then incubated at 37° C. for 16 h. Proteins in the processed samples were detected by immunoblot assay, as described above.

S1-ACE2 interaction assay.

A Takara Capturem IP and Co-IP kit (Cat No: 635721) was used to assess the affinity of SARS-CoV-2 S1 expressed by rSA11 viruses for ACE2. Protein A spin columns and all necessary buffers were included in the Capturem kit. MA104 cell monolayers were mock-infected or infected with rSA11 viruses (5 PFU/cell). At 9 h.p.i., the cells were washed and scraped into PBS, pelleted by low-speed centrifugation, and resuspended in Lysis/Equilibration Buffer containing protease inhibitor cocktail. After a 15 min incubation on ice, the lysate was clarified by centrifugation at 17,000 g for 10 min. Soluble hACE2-Fc (fchace2, InvivoGen), a recombinant protein consisting of the extracellular domain of human ACE2 fused to a human IgG1 Fc region, was added to the clarified lysates, to a final concentration of 20 mg per ml, and the mixture incubated overnight at 4° C. To recover complexes formed between the hACE2-Fc and S1 proteins, lysate samples were loaded onto pre-equilibrated protein A spin columns, which were then centrifuged at 1000 g for 1 min at room temperature. After rinsing columns with Wash Buffer, proteins were eluted from columns by adding Elution Buffer and centrifugation at 1000×g for 1 min at room temperature. The eluted samples were immediately neutralized by adding Neutralization Buffer. Proteins in eluted samples were detected by immunoblot assay, as described above.

### Immunofluorescence Assay

### Genetic Stability.

The genetic stability of recombinant RVs was assessed by serial passage on MA104-cell monolayers using 1:10 dilutions of infected cell lysates prepared in serum-free DMEM and 0.5 ug/ml trypsin (Philip and Patton, 2021). Viral dsRNA was recovered by Trizol extraction from clarified cell lysates treated with RNase TI to remove single-stranded RNA (Philip et al., 2019). Viral dsRNA was analyzed by electrophoresis on 8% polyacrylamide gels and detected by straining with ethidium bromide.

### GenBank Accession Numbers.

Modified segment 7 sequences of rSA11 viruses that have been deposited in GenBank: rSA11/wt. (LC178572), rSA11/NSP3-2A-3f-S1 (MW059026), rSA11/NSP3-2A-S1-1f (MZ511690), and rSA11/NSP3-2A-3f-S1-His (MZ511689). Other accession numbers include the SARS-CoV-2 S sequence in pTWIST/COVID19spike (GenBank MN908947), sequence for the African swine fever virus capping enzyme in pCMV-NP868R, and modified segment 7 RNA of rSA11-NSP3-P2A-3fUnaG (MK851042).

### Results

### Generation of Recombinant Viruses Encoding S1 Protein.

In a previous study, a rSA11 virus (rSA11/NSP3-2A-fS1) was generated with a modified segment 7 RNA that encoded the SARS-CoV-2 S1 protein with a fused N-terminal 3×FLAG tag (3×FLAG-S1). To understand the basis for the poor S1 expression by this virus, two similar rSA11 viruses differing only in nature of peptide tags encoded upstream and downstream of the S1 ORF in the segment 7 RNA were generated. One of the viruses, rSA11/NSP3-2A-fS1-His, was identical to rSA11/NSP3-2A-fS1, with the exception that the ORF in its segment 7 RNA was engineered to place a 6×His tag at the end of the S1 product, and thus encode 3×FLAG-S1-6×His. The rSA11/NSP3-2A-fS1-His virus was generated to address the possibility that, due to cleavage of the signal peptide from the S1 product, the N-terminal 3×FLAG tag was lost, preventing accurate assessment of fS1 synthesis by the rSA11/NSP3-2A-fS1 virus via immunoblot assay with anti-FLAG antibody. Instead, the production of S1 products could be assessed with anti-6×His antibody. The second recombinant virus that was made, rSA11/NSP3-2A-S1f, contained a segment 7 RNA designed to express S1 with a C-terminal 1×FLAG tag, but without any N-terminal tag (S1-1×FLAG). The usefulness of this virus was in examining the possibility that a tag positioned upstream of the S1 signal peptide might impede synthesis and glycosylation of the S1 protein by the endoplasmic reticulum.

The recombinant viruses, rSA11/NSP3-2A-S1f and rSA11/NSP3-2A-fS1-His, were produced following the same reverse genetics procedure used previously to generate rSA11/NSP3-2A-fS1-His and the wildtype virus, rSA11/wt. The procedure included transfection of BHK-T7 cells with a set of T7 transcription vectors (pT7) expressing SA11 plus-sense (+) RNAs and a CMV expression plasmid (pCMV-NP868R) encoding the capping enzyme of African swine fever virus. In the transfection mixtures, T7 transcription vectors for NSP2 and NSP5+RNAs (pT7/SA11NSP2 and pT7/SA11NSP5, respectively) were used as levels 3-fold greater that the other pT7 vectors. The modified segment 7 transcription vectors (FIG. 11) used in generating rSA11 encoding S1 products was added to transfection mixtures in place of pT7/NSP3SA11. Recombinant viruses formed in transfected BHK-T7 cells were amplified by overseeding with MA104 cells and then isolated by plaque purification.

Genomes and Growth Characteristics of rSA11s.

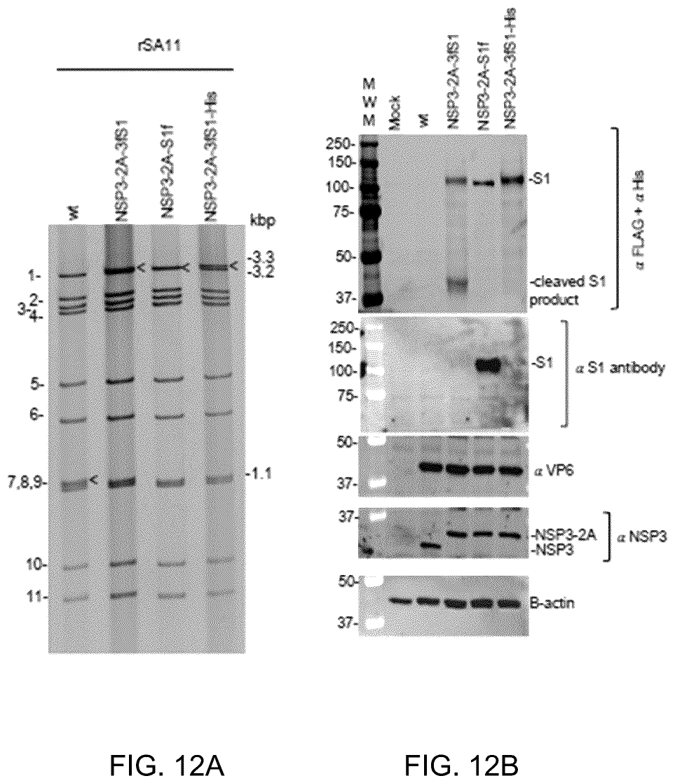

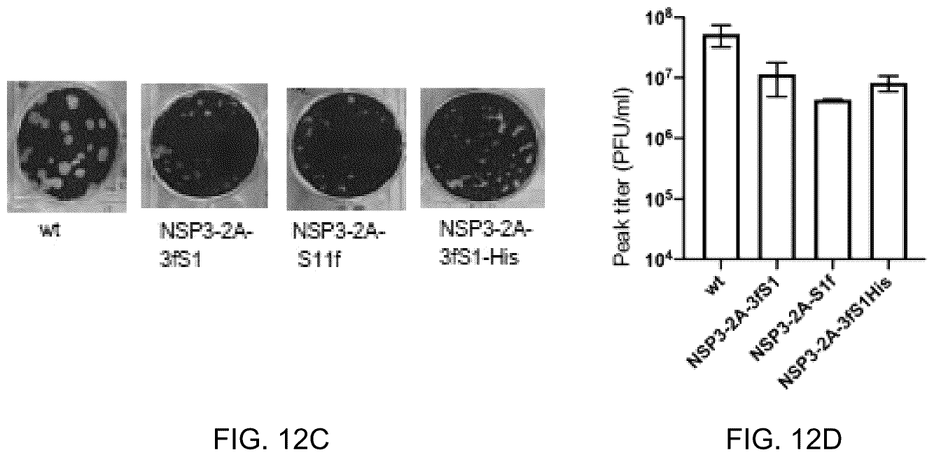

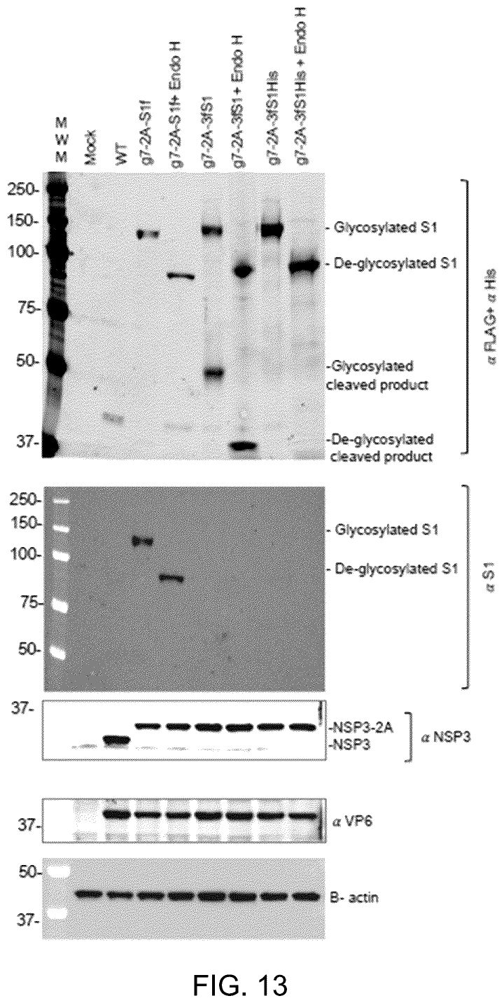

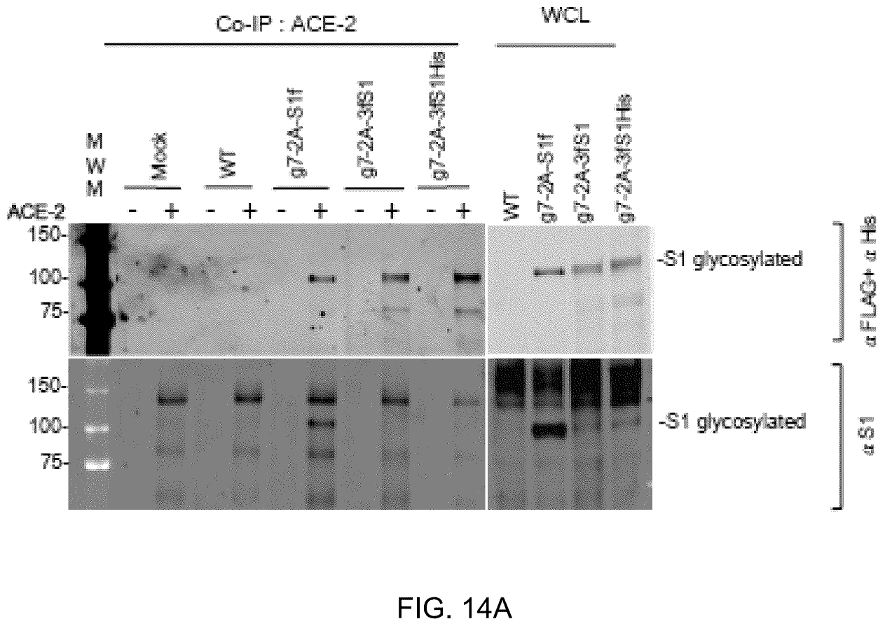

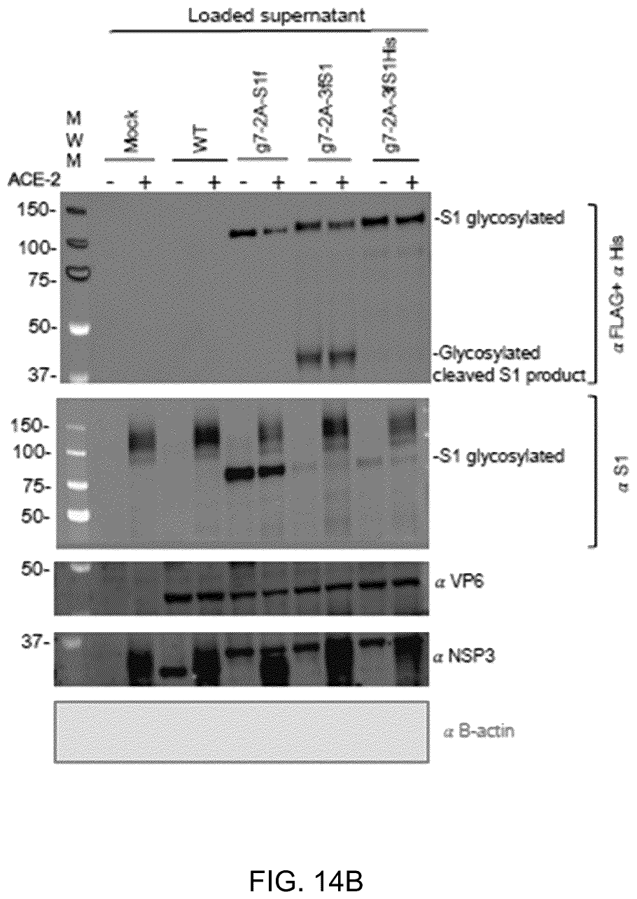

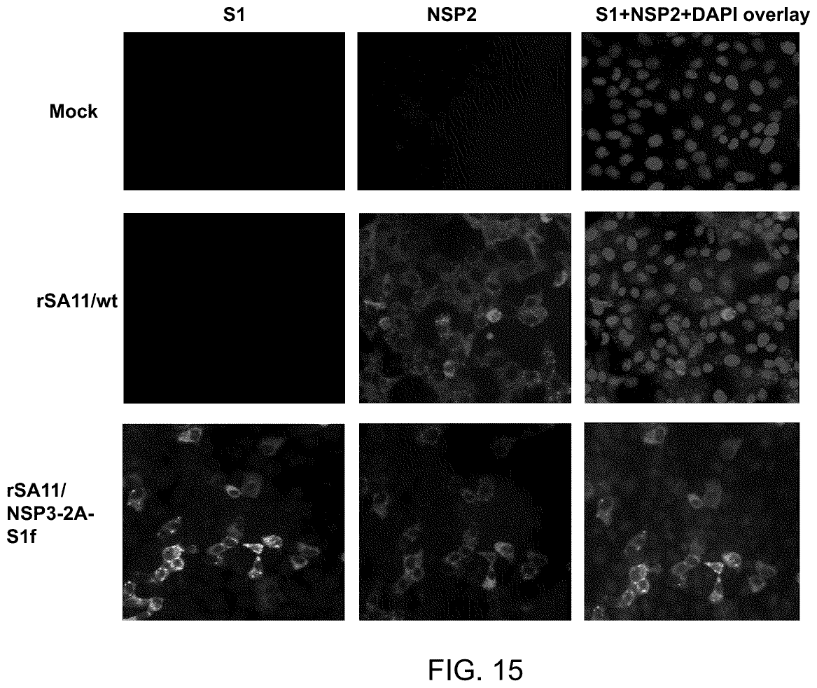

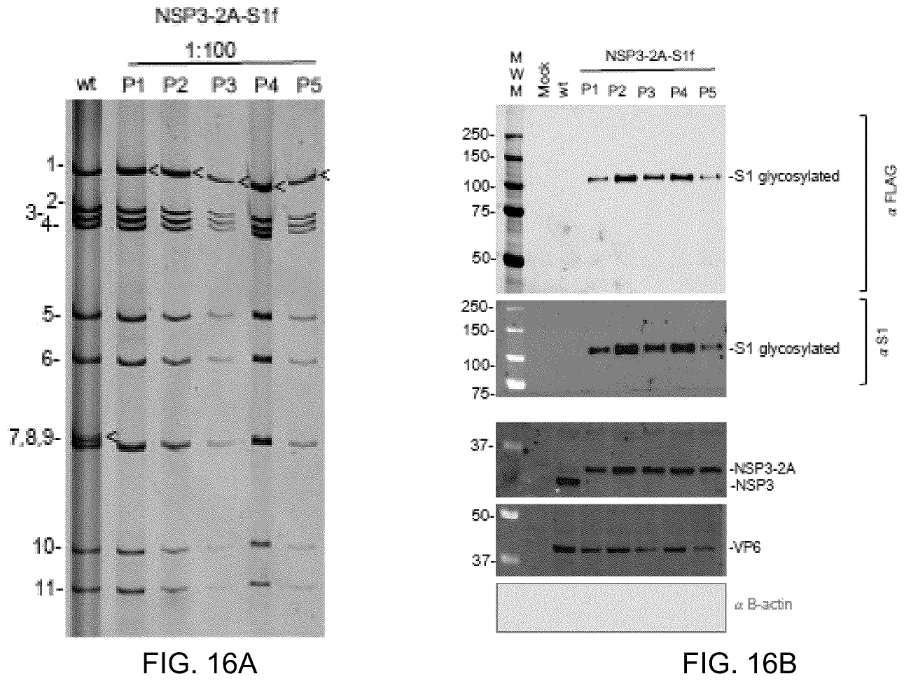

The dsRNA genome segments of recombinant viruses were resolved by gel electrophoresis to verify the presence of modified segment 7 RNAs (FIG. 12). The analysis showed that rSA11/NSP3-2A-fS1, rSA11/NSP3-2A-S1f and rSA11/NSP3-2A-fS1-His all lacked the 1.1-Kbp segment 7 dsRNA typical of rSA11/wt. Instead, as expected, the S1-encoding rSA11 viruses all contained segment 7 dsRNAs that migrated on polyacrylamide gels close to the position of the segment 1 dsRNA and had a size of 3.3 Kbp. Sequencing verified that the segment 7 RNA of the recombinant viruses was identical to that of the pT7 transcription vector used in their recovery. The total size of genome segments in each rSA11/NSP3-2A-fS1, rSA11/NSP3-2A-S1f and rSA11/NSP3-2A-fS1-His is 20.7-20.8 Kbp, which is the 2.1-2.2 Kbp (or ˜11%) greater than wildtype virus.

rSA11 Viruses Expressing the Glycosylated S1 Protein of SARS-CoV-2.

Followed by a second round of amplification on MA104 cells. Recombinant viruses were plaque purified from the virus pool and translation of the S1 RNA by the N-terminal 3×FLAG tag of the he fused to the fS1. In this design, the 3×FLAG tag was positioned immediately upstream of the S1 signal sequence (SS), an element critical for synthesis of glycosylated S1. In the previous study, the nature of S1 products expressed by rSA11/NSP3-2A-fS1 in infected cells was not certain, possibly due to instability or cleavage of S1 product or the impact of the FLAG tag on the function of SS sequence. This study has re-examined S1 products made by the rSA11/NSP3-2A-fS1 virus and compared its products to those generated by newly designed rSA11 viruses encoding S1 proteins lacking N-terminal tag sequences. The analysis shows that the newly design rSA11 viruses efficiently expressed glycosylated S1 proteins with the ability to bind the extracellular domain of ACE2. These results demonstrate that the RV segment 7-expression platform can be used to direct the expression of glycosylated capsid proteins.

While the disclosed subject matter is amenable to various modifications and alternative forms, specific embodiments are described herein in detail. The intention, however, is not to limit the disclosure to the particular embodiments described. The disclosure is intended to cover all modifications, equivalents, and alternatives falling within the scope of the disclosure as defined by the appended claims.

Similarly, although illustrative methods may be described herein, the description of the methods should not be interpreted as implying any requirement of, or particular order among or between, the various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As the terms are used herein with respect to ranges, “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

TABLE 1
Primer names and corresponding sequence

| SEQ | | |
|---|---|---|
| ID. | Primer | |
| NO. | Name | Sequence |
| 32 | Vector_For | CCATTTTGATACATGTTGAACAATCAAATACAGTGT |
| 33 | Vector_Rev | GCTAGCCTTGTCATCGTCATCCT |
| 34 | fP2_For | GATGACAAGGCTAGCACTACCCAGCTGTCAGCTG |
| 35 | fP2_Rev | CATGTATCAAAATGGTCACAGGTGCACATTATGACCAGTTCT |
| 36 | fP_For | GATGACAAGGCTAGCAAACCATTCACCGTCCCAATCT |
| 37 | fP_Rev | CATGTATCAAAATGGTTATAATGCACGCCTGCGCCC |
| 38 | fVP1_For | GATGACAAGGCTAGCATGAAGATGGCGTCGAGTGAC |
| 39 | fVP1_Rev | CATGTATCAAAATGGTTATAATGCACGCCTGCGCCC |
| 40 | Vector | CCATTTTGATACATGTTGAACAATCAAATACAGTGT |
| | P2A_For | |
| 41 | Vector | AGGACCGGGGTTTTCTTCCAC |
| | P2A_Rev | |
| 42 | VP1-fFor | GAAAACCCCGGTCCTGCTAGCGTGAAGATGGCGTCGAG |
| 43 | VP1-fRev | CATGTATCAAAATGGTTACTTGTCATCGTCATCCTTGTAATCTAATGCACGCCTGCGC |
| 44 | P His_For | AGAAAACCCCGGTCCTGCTAGCAAACCATTCACCGTCC |
| 45 | P His_Rev | CATGTATCAAAATGGTTAGTGGTGGTGGTGGTGGTGTAATGCACGCCTGCGCC |
| 46 | VP1- | GAAAACCCCGGTCCTGTGAAGATGGCGTCGAGTGAC |
| | ThHisFor | |
| 47 | VP1- | ACATGTATCAAAATGGTTAGTGGTGGTGGTGGTGGT |
| | ThHisRev | GCGAGCCACGGGGGACCAATAATGCACGCCT |
| 48 | VP1-HisFor | GAAAACCCCGGTCCTGTGAAGATGGCGTCGAGTGAC |
| 49 | VP1-HisRev | CATGTATCAAAATGGTTAGTGGTGGTGGTGGTGGTGTAATGCACGCCT |

TABLE 2
Properties of recombinant rRV/NSP3-2A-NoV strains.

| Virus strain | | | | | | | | |
|---|---|---|---|---|---|---|---|---|
| | | Genome | Genome segment 7 | | | | | |
| | | size/ | | Protein product | | | | |
| | | increase | | | 2A | | 2A | NCBI |
| Abbreviated | | over wt | RNA | uncleaved | cleaved | uncleaved | cleaved | accession |
| name | Formal name* | (bp) | (bp) | (aa) | (aa) | (kDa) | (kDa) | # |
| rSA11/ | RVA/Simian- | 18,559/ | 1105 | 315 | nd | 36.4 | nd | LC178572 |
| wt | lab/USA/SA11wt/2019/ | 0 | | | | | | |
| | G3P[2] | | | | | | | |
| rSA11/ | RVA/Simian- | 19,126/ | 1672 | 504 | 336 + | 57.1 | 38.5 + | MN190002 |
| NSP3- | lab/USA/SA11(NSP3-2A- | 567 | | | 168 | | 18.6 | |
| fP2 | NoV:fP2)/2019/ | | | | | | | |
| | G3P[2] | | | | | | | |
| rSA11/ | RVA/Simian- | 19,642/ | 2188 | 676 | 336 + | 76.1 | 38.5 + | MN190003 |
| NSP3- | lab/USA/SA11(NSP3-2A- | 1083 | | | 340 | | 37.7 | |
| fP | NoV:fP)/2019/ | | | | | | | |
| | G3P[2] | | | | | | | |
| rSA11/ | RVA/Simian- | 20,314/ | 2860 | 900 | 336 + | 100.3 | 38.5 + | MN190004 |
| NSP3- | lab/USA/SA11(NSP3-2A- | 1755 | | | 564 | | 61.9 | |
| fVP1 | NoV:fVP1)/2019/ | | | | | | | |
| | G3P[2] | | | | | | | |
| rSA11/ | RVA/Simian- | 20.272/ | 2818 | 886 | 336 + | 98.6 | 38.5 + | MN201548 |
| NSP3- | lab/USA/SA11(NSP3-2A- | 1713 | | | 550 | | 60.1 | |
| VP1f | NoV:VP1f)/2019/ | | | | | | | |
| | G3P[2] | | | | | | | |
| rSA11/ | RVA/Simian- | 19,594/ | 2140 | 660 | 336 + | 74.2 | 38.5 + | MN201549 |
| NSP3- | lab/USA/SA11(NSP3-2A- | 1035 | | | 324 | | 35.8 | |
| PHis | NoV:PHis)/ | | | | | | | |
| | 2019/G3P[2] | | | | | | | |
| rSA11/ | RVA/Simian- | 20,278/ | 2824 | 888 | 336 + | 98.8 | 38.5 + | MN201547 |
| NSP3- | lab/USA/SA11(NSP3-2A- | 1719 | | | 552 | | 60.4 | |
| Th-VP1 | NoV:VP1ThHis)/ | | | | | | | |
| His | 2019/G3P[2] | | | | | | | |
| rSA11/ | RVA/Simian- | 20,260/ | 2806 | 882 | 336 + | 98.2 | 38.5 + | MZ562305 |
| NSP3- | lab/USA/SA11(NSP3-2A- | 1701 | | | 546 | | 59.8 | |
| VP1 His | NoV:VP1His)/ | | | | | | | |
| | 2019/G3P[2] | | | | | | | |
| rSA11/ | RVA/Simian- | 19,563/ | 2109 | 655 | 331 + | 74.2 | 38 + | MZ643978 |
| RIX | lab/USA/SA11(RIX | 1004 | | | 324 | | 35.8 | |
| NSP3- | NSP3-2A-NoV:PHis)/ | | | | | | | |
| PHis | 2021/G3P[2] | | | | | | | |

*Formal strain names were assigned according to Matthijnssens et al (2011).
nd: not determined, no 2A cleavage site present;
wt: wildtype

TABLE 3
Characteristic of variant and detail of sequence duplication and/or deletion

| | | | | | | | | | Details of | | Details of | |
|---|---|---|---|---|---|---|---|---|---|---|---|---|
| | | | | | | | | | sequence | | sequence | |
| Characteristics of unstable variant segments | | | | | | | | | duplication | | deletion | |
| Name | Size | Number of bp in each region | | | | | | | | Region | | Nature |
| of | of | | | 2A | | NoVVP1 | | 3′- | Sequence | of | Sequence | of |
| genome | dsRNA | 5′-UTR | NSP3ORF | peptide | Flag | sequence | His | UTR | duplication | sequence | deletion | sequence |
| segment | segment | (bp) | (bp) | (bp) | (bp) | (bp) | (bp) | (bp) | (bp) | duplication | (bp) | deletion |
| rSA11/ | 1105 | 25 | 948 | 0 | 0 | 0 | 0 | 132 | 0 | | 0 | |
| wt | | | | | | | | | | | | |
| rSA11/ | 2860 | 25 | 945 | 66 | 66 | 1626 | 0 | 132 | 0 | | 0 | |
| NSP3- | | | | | | | | | | | | |
| fVP1 | | | | | | | | | | | | |
| fVP1/ | 1550 | 25 | 945 | 66 | 66 | 36 | 0 | 125 | 287 | 287 bp | 7 | 7 bp |
| R1 | | | | | | | | | | (NSP3ORFand | | (3′- |
| | | | | | | | | | | 2A) | | UTR) |
| fVP1/ | 1263 | 25 | 945 | 66 | 66 | 36 | 0 | 125 | 0 | | 7 | 7 bp |
| R2 | | | | | | | | | | | | (3′- |
| | | | | | | | | | | | | UTR) |
| fVP1/V1/ | 1551 | 25 | 945 | 66 | 66 | 308 | 0 | 132 | 9 | | 0 | |
| R1 | | | | | | | | | | | | |
| fVP1/V1/ | 1417 | 25 | 945 | 66 | 66 | 36 | 0 | 125 | 154 | 154 bp | 7 | 7 bp |
| R2 | | | | | | | | | | (NSP3ORF | | (3′ |
| | | | | | | | | | | and 2A) | | UTR) |
| fVP1/V1/ | 1263 | 25 | 945 | 66 | 66 | 36 | 0 | 125 | 0 | | 7 | 7 bp |
| R3 | | | | | | | | | | | | (3′- |
| | | | | | | | | | | | | UTR) |
| fVP1/V3/ | 1167 | 25 | 945 | 25 | 0 | 40 | 0 | 132 | 0 | | 0 | |
| R | | | | | | | | | | | | |
| fVP1/V4/ | 1320 | 25 | 945 | 66 | 66 | 77 | 0 | 132 | 9 | 9 bp | 0 | |
| R1 | | | | | | | | | | (3′- | | |
| | | | | | | | | | | UTR) | | |
| fVP1/V4/ | 1110 | 25 | 940 | 0 | 0 | 8 | 0 | 132 | 5 | 5 bp | 0 | |
| R3 | | | | | | | | | | (NSP3ORF) | | |
| rSA11/ | 2806 | 25 | 945 | 66 | 0 | 1620 | 18 | 132 | 0 | | 0 | |
| NSP3- | | | | | | | | | | | | |
| VP1 | | | | | | | | | | | | |
| His | | | | | | | | | | | | |
| VP1His/ | 1259 | 25 | 945 | 22 | 0 | 99 | 18 | 132 | 18 | 18 bp | 0 | |
| R | | | | | | | | | | (NSP3ORF) | | |
| VP1His/ | 1849 | 25 | 945 | 66 | 0 | 621 | 16 | 132 | 44 | 44 bp | 0 | |
| V5/R1 | | | | | | | | | | (NSP3ORF) | | |
| VP1His/ | 1485 | 25 | 945 | 66 | 0 | 281 | 18 | 132 | 18 | 18 bp | 0 | |
| V5/R2 | | | | | | | | | | (VP1) | | |
| VP1His/ | 1038 | 25 | 922 | 0 | 0 | 0 | 0 | 91 | 0 | | 64 | 23 bp |
| V5/R3 | | | | | | | | | | | | (NSP3ORF), |
| | | | | | | | | | | | | 41 bp |
| | | | | | | | | | | | | (3′- |
| | | | | | | | | | | | | UTR) |
| VP1His/ | 1087 | 25 | 936 | 0 | 0 | 0 | 0 | 126 | 0 | | 15 | 9 bp |
| V6/R | | | | | | | | | | | | (NSP3ORF), |
| | | | | | | | | | | | | 6 bp |
| | | | | | | | | | | | | (3′- |
| | | | | | | | | | | | | UTR) |
| VP1His/ | 1121 | 25 | 945 | 2 | 0 | 3 | 0 | 132 | 14 | 14 bp | 0 | |
| V7/R | | | | | | | | | | (NSP3ORF) | | |

1. A polynucleotide comprising:
a sequence encoding a rotavirus (RV) NSP3 protein; and
a heterologous polynucleotide.

2-5. (canceled)

6. The polynucleotide of claim 1, wherein the sequence encoding an RV NSP3 is a sequence encoding SEQ ID NO: 1, or a sequence with at least about 90% identity to SEQ ID NO: 1.

7-12. (canceled)

13. The polynucleotide of claim 12, wherein the peptide or protein comprises a norovirus (NoV) peptide or protein.

14. The polynucleotide of claim 13, wherein the NoV peptide or protein comprises NoV VP1 protein.

15. The polynucleotide of claim 14, wherein the NoV peptide or protein is selected from SEQ ID NOs: 24, 26 or 80-84.

16. The polynucleotide of claim 12, wherein the peptide or protein comprises a SARS-CoV-2 protein or peptide.

17. The polynucleotide of claim 16, wherein the SARS-CoV-2 protein or peptide is selected from an N protein, an S protein, or fragment of either an N protein or an S protein.

18. The polynucleotide of claim 17, wherein the SARS-CoV-2 protein is an S1 protein or a fragment thereof.

19-21. (canceled)

22. The polynucleotide of claim 20, wherein the cleavage site is a self-cleaving peptide sequence.

23. The polynucleotide of claim 22, wherein the self-cleaving peptide sequence is porcine teschovirus 2A element (SEQ ID NO: 29).

24-25. (canceled)

26. The polynucleotide of claim 1, wherein the heterologous polynucleotide is about 2.6 kb or less in length.

27-28. (canceled)

29. The polynucleotide of claim 7, wherein the peptide or protein is a glycoprotein.

30-33. (canceled)

34. A polynucleotide encoding a protein selected from SEQ ID NOs: 2-11, or a sequence with at least about 90% identity to a sequence selected from SEQ ID NOs: 2-11.

35. A polynucleotide comprising any one of SEQ ID NOs: 13-22 or a sequence with at least about 90% identity to any one of SEQ ID NOs: 13-22.

36. An infectious particle comprising the polynucleotide of claim 1.

37. An infectious particle made by introducing the polynucleotide of claim 1 into a cell.

38. A pharmaceutical composition comprising the infectious particle of claim 36.

39. A method of eliciting an immune response to one or more microorganism in a subject, the method comprising: administering an effective amount of the pharmaceutical composition of claim 38 to a subject to elicit an immune response to the one or more microorganism.

40-43. (canceled)

44. A method of vaccinating a subject against one or more pathogens, the method comprising: administering an effective amount of the pharmaceutical composition of claim 38 to a subject to vaccinate a subject against the one or more pathogens.

45-48. (canceled)

49. A method of generating recombinant rotavirus (RV) in vitro comprising:
introducing the polynucleotide of claim 1 into a cell; allowing the cell to express the polynucleotide; incubating the cells for a sufficient time to produce RV; and harvesting virus produced by the cells to generate RV in vitro.

50-58. (canceled)

59. A cell comprising the composition of claim 1.

60-62. (canceled)

63. A system, platform, or kit for generating recombinant rotavirus (RV) comprising:
(a) the polynucleotide of claim 1; and
(b) cells capable of expressing the polynucleotides of (a).

64-69. (canceled)
